SEQUENCE LISTING (31 sequences)

SEQ ID NO: 1 - protein, 115 aa, Homo sapiens [sequence omitted]

SEQ ID NO: 2 - protein, 112 aa, Homo sapiens
  MISC_FEATURE (1)..(112): ABT-874 Light Chain Variable Region
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

SEQ ID NO: 3 - protein, 306 aa, Homo sapiens
  MISC_FEATURE (1)..(306): mature form, human IL-12 P40 subunit. Swiss-Prot P29460
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 197 aa, Homo sapiens
  MISC_FEATURE (1)..(197): mature form, human IL-12 P35 subunit. Swiss-Prot P29459
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 328 aa, Homo sapiens
  MISC_FEATURE (1)..(328): Precursor p40 Peptide - sp|P29460|IL12B_HUMAN Interleukin-12 subunit beta OS=Homo sapiens GN=IL12B PE=1 SV=1
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 6 - protein, 219 aa, Homo sapiens
  MISC_FEATURE (1)..(219): Precursor p35 Peptide - sp|P29459|IL12A_HUMAN Interleukin-12 subunit alpha OS=Homo sapiens GN=IL12A PE=1 SV=2
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - DNA, 25 bp, Artificial Sequence
  Synthetic
caccatgggtcaccagcagttggtc

SEQ ID NO: 8 - DNA, 47 bp, Artificial Sequence
  Synthetic
accctggaagtacaggttttcactgcagggcacagatgcccattcgc

SEQ ID NO: 9 - DNA, 35 bp, Artificial Sequence
  Synthetic
cgtagaattggattggcgtccggatgcccctggag

SEQ ID NO: 10 - DNA, 35 bp, Artificial Sequence
  Synthetic
ctccaggggcatccggacgccaatccaattctacg

SEQ ID NO: 11 - DNA, 37 bp, Artificial Sequence
  Synthetic
ctgcttcacaaaaaggaaaacggaatttggtccactg

SEQ ID NO: 12 - DNA, 37 bp, Artificial Sequence
  Synthetic
cagtggaccaaattccgttttcctttttgtgaagcag

SEQ ID NO: 13 - DNA, 40 bp, Artificial Sequence
  Synthetic
gatggaatttggtccactgagattttaaaggaccagaaag

SEQ ID NO: 14 - DNA, 40 bp, Artificial Sequence
  Synthetic
ctttctggtcctttaaaatctcagtggaccaaattccatc

SEQ ID NO: 15 - DNA, 58 bp, Artificial Sequence
  Synthetic
ggtccactgatattttaaagaaccagaaagaattcaaaaataagacctttctaagatg

SEQ ID NO: 16 - DNA, 58 bp, Artificial Sequence
  Synthetic
catcttagaaaggtcttatttttgaattctttctggttctttaaaatatcagtggacc

SEQ ID NO: 17 - DNA, 40 bp, Artificial Sequence
  Synthetic
gacacccctgaagaagatgacatcacctggaccttggacc

SEQ ID NO: 18 - DNA, 40 bp, Artificial Sequence
  Synthetic
ggtccaaggtccaggtgatgtcatcttcttcaggggtgtc

SEQ ID NO: 19 - DNA, 60 bp, Artificial Sequence
  Synthetic
gatggtatcacctggacctccgaccagcgccggggggtcatcggctctggcaaaaccctg

SEQ ID NO: 20 - DNA, 60 bp, Artificial Sequence
  Synthetic
ggtcagggttttgccagagccgatgaccccccggcgctggtcggaggtccaggtgatacc

SEQ ID NO: 21 - DNA, 56 bp, Artificial Sequence
  Synthetic
gctgctacactctctgcagagaaggtcaccctgaaccagcgtgactatgagtactc

SEQ ID NO: 22 - DNA, 54 bp, Artificial Sequence
  Synthetic
ggcactccactgagtactcatagcacgctggttcagggtgaccttctctgcaga

SEQ ID NO: 23 - DNA, 49 bp, Artificial Sequence
  Synthetic
ggtccactgatattttaaagaacttcaaaaataagacctttctaagatg

SEQ ID NO: 24 - DNA, 49 bp, Artificial Sequence
  Synthetic
catcttagaaaggtcttatttttgaagttctttaaaatatcagtggacc

SEQ ID NO: 25 - DNA, 45 bp, Artificial Sequence
  Synthetic
gtccactgatattttaaaggaccccaaaaataagacctttctaag

SEQ ID NO: 26 - DNA, 45 bp, Artificial Sequence
  Synthetic
cttagaaaggtcttatttttggggtcctttaaaatatcagtggac

SEQ ID NO: 27 - protein, 160 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRG

SEQ ID NO: 28 - protein, 160 aa, Rhesus
IWELKKDVYVVELDWYPDAPGEMVVLTCDTPEEDGITWTLDQSGEVLGSGKTLTIQVKEFGDAGQYTCHKGGEALSHSLLLLHKKEDGIWSTDVLKDQKEPKNKTFLRCEAKNYSGRFTCWWLTTISTDLTFSVKSSRGSSNPQGVTCGAVTLSAERVRG

SEQ ID NO: 29 - protein, 160 aa, Dog
IWELEKDVYVVELDWHPDAPGEMVVLTCHTPEEDDITWTSAQSSEVLGSGKTLTIQVKEFGDAGQYTCHKGGKVLSRSLLLIHKKEDGIWSTDILKEQKESKNKIFLKCEAKNYSGRFTCWWLTAISTDLKFSVKSSRGFSDPQGVTCGAVTLSAERVRV

SEQ ID NO: 30 - protein, 157 aa, Rat
MWELEKDVYVVEVDWRPDAPGETVTLTCDSPEEDDITWTSDQRRGVIGSGKTLTITVREFLDAGQYTCHRGGETLSHSHLLLHKKENGIWSTEILKNFKNKTFLKCEAPNYSGRFTCSWLVHRNTDLKFNIKSSSSSPESRAVTCGRASLSAEKVTL

SEQ ID NO: 31 - protein, 157 aa, Mouse
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTL

(19) United States
(12) Patent Application Publication
(10) Pub. No.: US 2012/0288494 A1
(43) Pub. Date: Nov. 15, 2012

(54) Anti-IL-12/IL-23 antibodies and uses thereof

(75) Inventors: David W. Borhani, Hartsdale, NY (US); Ramkrishna Sadhukhan, Acton, MA (US); Susan E. Lacy, Westborough, MA (US); Holly H. Soutter, Shrewsbury, MA (US)

(73) Assignee: Abbott Laboratories, Abbott Park, IL (US)

(21) Appl. No.: 13/374,693

(22) Filed: Jan. 6, 2012

Related U.S. Application Data
(63) Continuation-in-part of application No. 12/121,615, filed on May 15, 2008, now abandoned.
Application No. 12/121,615 is a division of application No. 10/884,830, filed on Jul. 1, 2004, now Pat. No. 7,504,485.
Application No. 10/884,830 is a division of application No. 09/534,717, filed on Mar. 24, 2000, now Pat. No. 6,914,128.
(60) Provisional application No. 61/460,780, filed on Jan. 7, 2011.
(60) Provisional application No. 60/126,603, filed on Mar. 25, 1999.

(51) Int. Cl. C07K 16/24; C12N 15/13; A61P 25/00; A61P 17/06; A61P 29/00; A61P 19/02; A61P 1/04; A61P 37/06; A61P 37/00; A61P 35/00; C12N 15/63; C12N 5/10; C12N 1/00; G01N 33/566; C07K 1/00; A61K 39/395

(52) U.S. Cl. 424/133.1; 530/387.9; 530/387.3; 424/139.1; 536/23.53; 435/320.1; 435/331; 435/328; 435/243; 435/7.92; 436/501; 435/7.1

(57) ABSTRACT

The present invention provides antibodies, and antigen-binding portions thereof, that bind to epitopes comprising at least one amino acid residues from residues 1-197 of the p40 subunit of IL-12 and/or IL-23. The invention further provides nucleic acids encoding the antibodies, compositions, vectors and host cells comprising the antibodies, and methods of making and using the same.

### RELATED APPLICATIONS

This application claims priority to U.S. provisional application No. 61/460,780, filed on Jan. 7, 2011. This application is also a continuation-in-part of U.S. patent application Ser. No. 12/121,615, filed May 15, 2008, which is a divisional of U.S. patent application Ser. No. 10/884,830, filed Jul. 1, 2004, now U.S. Pat. No. 7,504,485; which is a divisional of U.S. patent application Ser. No. 09/534,717, filed Mar. 24, 2000, now U.S. Pat. No. 6,914,128; which is a non-provisional application claiming priority to U.S. provisional application Ser. No. 60/126,603, filed Mar. 25, 1999. The entire contents of each of the foregoing applications are hereby incorporated herein by reference.

### BACKGROUND OF THE INVENTION

Human interleukin 12 (IL-12) has been characterized as a cytokine with a unique structure and pleiotropic effects (Kobayashi, et al. (1989) J. Exp Med. 170:827-845; Seder, et al. (1993) Proc. Natl. Acad. Sci. 90:10188-10192; Ling, et al. (1995) J. Exp Med. 154:116-127; Podlaski, et al. (1992) Arch. Biochem. Biophys. 294:230-237). IL-12 plays a critical role in the pathology associated with several diseases involving immune and inflammatory responses. A review of IL-12, its biological activities, and its role in disease can be found in Gately et al. (1998) Ann. Rev. Immunol. 16: 495-521.

Structurally, IL-12 is a heterodimeric protein comprising a 35 kDa subunit (p35) and a 40 kDa subunit (p40) which are both linked together by a disulfide bridge (referred to as the “p70 subunit”). The heterodimeric protein is produced primarily by antigen-presenting cells such as monocytes, macrophages and dendritic cells. These cell types also secrete an excess of the p40 subunit relative to p70 subunit. The p40 and p35 subunits are genetically unrelated and neither has been reported to possess biological activity, although the p40 homodimer may function as an IL-12 antagonist.

Functionally, IL-12 plays a central role in regulating the balance between antigen specific T helper type (Th1) and type 2 (Th2) lymphocytes. The Th1 and Th2 cells govern the initiation and progression of autoimmune disorders, and IL-12 is critical in the regulation of Th1-lymphocyte differentiation and maturation. Cytokines released by the Th1 cells are inflammatory and include interferon gamma (IFNγ), IL-2 and lymphotoxin (LT). Th2 cells secrete IL-4, IL-5, IL-6, IL-10 and IL-13 to facilitate humoral immunity, allergic reactions, and immunosuppression. Consistent with the preponderance of Th1 responses in autoimmune diseases and the proinflammatory activities of IFNγ, IL-12 may play a major role in the pathology associated with many autoimmune and inflammatory diseases such as rheumatoid arthritis (RA), multiple sclerosis (MS), and Crohn's disease.

Human patients with MS have demonstrated an increase in IL-12 expression as documented by p40 mRNA levels in acute MS plaques. (Windhagen et al., (1995) J. Exp. Med. 182: 1985-1996). In addition, ex vivo stimulation of antigen-presenting cells with CD40L-expressing T cells from MS patients resulted in increased IL-12 production compared with control T cells, consistent with the observation that CD40/CD40L interactions are potent inducers of IL-12.

Elevated levels of IL-12 p70 have been detected in the synovia of RA patients compared with healthy controls (Morita et al (1998) Arthritis and Rheumatism. 41: 306-314). Cytokine messenger ribonucleic acid (mRNA) expression profile in the RA synovia identified predominantly Th1 cytokines. (Bucht et al., (1996) Clin. Exp. Immunol. 103: 347-367). IL-12 also appears to play a critical role in the pathology associated with Crohn's disease (CD). Increased expression of IFNγ and IL-12 has been observed in the intestinal mucosa of patients with this disease (Fais et al. (1994) J. Interferon Res. 14:235-238; Parronchi et al., (1997) Am. J. Path. 150:823-832; Monteleone et al., (1997) Gastroenterology. 112:1169-1178, and Berrebi et al., (1998) Am. J. Path 152:667-672). The cytokine secretion profile of T cells from the lamina propria of CD patients is characteristic of a predominantly Th1 response, including greatly elevated IFNγ levels (Fuss, et al., (1996) J. Immunol. 157:1261-1270). Moreover, colon tissue sections from CD patients show an abundance of IL-12 expressing macrophages and IFNγ expressing T cells (Parronchi et al (1997) Am. J. Path. 150:823-832).

Due to the role of human IL-12 in a variety of human disorders, therapeutic strategies have been designed to inhibit or counteract IL-12 activity. In particular, antibodies that bind to, and neutralize, IL-12 have been sought as a means to inhibit IL-12 activity. The highly specific recognition of an antigen (Ag) allows antibodies (Ab) to mount the humoral immune response to foreign invaders and to discriminate between self and non-self. Monoclonal antibodies (mAb) have been developed for use as protein therapeutics in the treatment of various conditions, including autoimmune diseases (Brekke, O. H. and I. Sandlie (2003). “Therapeutic antibodies for human diseases at the dawn of the twenty-first century.” Nat Rev Drug Discov 2 (1): 52-62). Antibodies can act as therapeutics by neutralizing a disease-related target molecule or by targeting specific cells for destruction.

Interleukin 23 (IL-23) is a human heterodimeric cytokine protein that consists of two subunits, p19 (the IL-23 alpha subunit), and p40 which is the beta subunit of IL-12 (i.e., IL-12B). IL-23 is secreted by a number of different cells including macrophages and dendritic cells. IL-23, like IL-12, appears to be important in the development of autoimmune diseases; for example, it plays a key role in a murine model of multiple sclerosis (Cua, D. J., J. Sherlock, et al. (2003). “Interleukin-23 rather than interleukin-12 is the critical cytokine for autoimmune inflammation of the brain.” Nature 421 (6924): 744-8).

Some of the earliest antibodies were murine monoclonal antibodies (mAbs), secreted by hybridomas prepared from lymphocytes of mice immunized with IL-12 (see e.g., World Patent Application Publication No. WO 97/15327 by Strober et al.; Neurath et al. (1995) J. Exp. Med. 182:1281-1290; Duchmann et al. (1996) J. Immunol. 26:934-938). These murine IL-12 antibodies are limited for their use in vivo due to problems associated with administration of mouse antibodies to humans, such as short serum half life, an inability to trigger certain human effector functions and elicitation of an unwanted immune response against the mouse antibody in a human (the “human anti-mouse antibody” (HAMA reaction)).

In general, attempts to overcome the problems associated with use of fully-murine antibodies in humans, have involved genetically engineering the antibodies to be more “human-like.” For example, chimeric antibodies, in which the variable regions of the antibody chains are murine-derived and the constant regions of the antibody chains are human-derived, have been prepared (Junghans, et al. (1990) Cancer Res. 50:1495-1502; Brown et al. (1991) Proc. Natl. Acad. Sci. 88:2663-2667; Kettleborough et al. (1991) Protein Engineering. 4:773-783). However, because these chimeric and humanized antibodies still retain some murine sequences, they still may elicit an unwanted immune reaction, the human anti-chimeric antibody (HACA) reaction, especially when administered for prolonged periods. A preferred IL-12 inhibitory agent to murine antibodies or derivatives thereof (e.g., chimeric or humanized antibodies) would be an entirely human anti-IL-12 antibody, since such an agent should not elicit the HAMA reaction, even if used for prolonged periods.

Seventeen mAbs are approved for therapeutic use. Examples include murine mAbs (e.g. ORTHOCLONE OKT®3 (anti-CD3) for acute allograft rejection (Ortho Multicenter Transplant Study Group (1985). “A randomized clinical trial of OKT3 monoclonal antibody for acute rejection of cadaveric renal transplants. Ortho Multicenter Transplant Study Group.” N Engl J Med 313 (6): 337-42), murine-human chimeric mAbs in which murine variable domains are grafted onto human constant domains (e.g. Remicade® (anti-TNFα) for rheumatoid arthritis and Crohn's disease (Bondeson, J. and R. N. Maini (2001). “Tumour necrosis factor as a therapeutic target in rheumatoid arthritis and other chronic inflammatory diseases: the clinical experience with infliximab (REMICADE).” Int J Clin Pract 55 (3): 211-6), and Rituxan® (anti-CD20) for non-Hodgkin's lymphoma (White, C. A., R. L. Weaver, et al. (2001). “Antibody-targeted immunotherapy for treatment of malignancy.” Annu Rev Med 52: 125-45), humanized mAbs in which murine complementarity-determining regions (CDRs) are incorporated into an otherwise human immunoglobulin (e.g. Herceptin® (anti-Her2) for breast cancer (Shak, S. (1999). “Overview of the trastuzumab (Herceptin) anti-HER2 monoclonal antibody clinical program in HER2-overexpressing metastatic breast cancer. Herceptin Multinational Investigator Study Group.” Semin Oncol 26 (4 Suppl 12): 71-7), and, most recently, recombinant human mAbs (e.g. Humira® (anti-TNFα) for rheumatoid arthritis (Weinblatt, M. E., E. C. Keystone, et al. (2003). “Adalimumab, a fully human anti-tumor necrosis factor alpha monoclonal antibody, for the treatment of rheumatoid arthritis in patients taking concomitant methotrexate: the ARMADA trial.” Arthritis Rheum 48 (1): 35-45), wherein both the hypervariable and framework residues are drawn from naturally-occurring human immunoglobulin sequences.

The three-dimensional structures of therapeutic mAb are of considerable interest to both scientists and clinicians. The mAb binding affinity and specificity, and the kinetics of Ag binding and release, are all functional characteristics crucial to success or failure in the clinic. A fuller understanding of these characteristics follows from knowledge of the structures of a mAb and the mAb-Ag complex. An understanding of the structural basis for these properties also brings with it the power of rational optimization of antigen-binding molecules for therapeutic utility. Accordingly, there is an ongoing need for therapeutic agents, e.g., antibodies and antigen-binding proteins derived therefrom, that are optimized for binding to an antigen, e.g., the p40 subunit of IL-12 and IL-23. These antibodies will be effective in ameliorating the effects of aberrant IL-12 and/or IL-23 activity.

### SUMMARY OF THE INVENTION

The present invention is based, at least in part, on an x-ray crystallographic study of polypeptides comprising the antigen binding fragment (Fab) of the anti-p40 subunit of IL-12/IL-23 antibody J695, alone and complexed to the interleukin-12 (IL-12) p70 (hereinafter IL-12 p70, or simply IL-12). The atomic coordinates that result from this study are of use in identifying and designing improved antibodies and other antibody-like binding molecules (e.g., antibody fragments, or domain antibodies) that bind p40-containing cytokines such as IL-12 and IL-23. These improved antibodies are of use in methods of treating a patient having a condition which is modulated by or dependent upon the biological activity of p40-containing cytokines, including, for example, a condition dependent on inappropriate or undesired stimulation of the immune system (multiple sclerosis, psoriasis, rheumatoid arthritis, Crohn's disease, lupus erythromatosis, chronic inflammatory diseases, and graft rejection following transplant surgery) or cancer.

Accordingly, in one aspect, the present invention provides an isolated antibody or antigen-binding fragment thereof, that binds to the p40 subunit of IL-12 and/or IL-23, wherein the antibody or antigen-binding fragment thereof, binds to a portion and/or conformational epitope of the p40 subunit comprising at least one amino acid residue (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196 or 197 residues) selected from residues 1-197 of the amino acid sequence of SEQ ID NO: 3, or within 1-10 Å of the amino acid residue. In one embodiment, the invention provides an isolated antibody or antigen-binding fragment thereof, that binds to the p40 subunit of IL-12 and/or IL-23, wherein the antibody or antigen-binding fragment thereof, binds to a portion and/or conformational epitope of the p40 subunit comprising at least one amino acid residue selected from residues 1-107 of the amino acid sequence of SEQ ID NO: 3, or within 1-10 Å of the amino acid residue.

In another embodiment, the invention provides an isolated antibody, or antigen-binding portion thereof, that binds to the p40 subunit of IL-12 and/or IL-23, wherein the antibody binds to a portion and/or conformational epitope of the p40 subunit comprising at least one amino acid residue of loops 1-7 of the p40 subunit, and wherein the at least one amino acid residue is selected from the group consisting of residues 14-23, 58-60, 84-107, 124-129, 157-164 and 194-197 of the amino acid sequence of SEQ ID NO: 3, or within 1-10 Å of said amino acid residue.

In another embodiment, the invention provides an isolated antibody, or antigen-binding portion thereof, that binds to the p40 subunit of IL-12 and/or IL-23, wherein the antibody binds to a portion and/or conformational epitope of the p40 subunit comprising at least one amino acid residue of loops 1-7 of the p40 subunit, and wherein at least one amino acid residue is selected from the group consisting of residues Asp14, Trp15, Tyr16, Pro17, Asp18, Ala19, Pro20, Gly21, Glu22, Met23, Lys58, Glu59, Phe60, Lys84, Lys85, Glu86, Asp87, Gly88, Ile89, Trp90, Ser91, Thr92, Asp93, Ile94, Leu95, Lys96, Asp97, Gln98, Lys99, Glu100, Pro101, Lys102, Asn103, Lys104, Thr105, Phe106, Leu107, Thr124, Thr125, Ile126, Ser127, Thr128, Asp129, Arg157, Val158, Arg159, Gly160, Asp161, Asn162, Lys163, Glu164, His194, Lys195, Leu196 and Lys197 of the amino acid sequence of SEQ ID NO: 3, or within 1-10 Å of the amino acid residue.

In one embodiment, the isolated antibody, or antigen binding portion thereof, binds to a portion and/or conformational epitope of the p40 subunit comprising at least one amino acid residue of loop 1 selected from the group consisting of residues 14-23, or within 1-10 Å of said amino acid residue. In one embodiment, the isolated antibody, or antigen binding portion thereof, binds to a portion and/or conformational epitope of the p40 subunit comprising at least one amino acid residue of loop 1 selected from the group consisting of residues 14-18, or within 1-10 Å of said amino acid residue. In one embodiment, the isolated antibody, or antigen binding portion thereof, binds to a portion and/or conformational epitope of the p40 subunit comprising at least one amino acid residue of loop 1 selected from the group consisting of residues 14-17, or within 1-10 Å of said amino acid residue. In one embodiment, the isolated antibody, or antigen binding portion thereof, binds to a portion and/or conformational epitope of the p40 subunit comprising at least one amino acid residue of loop 1 selected from the group consisting of residues 15-17, or within 1-10 Å of said amino acid residue.

In one embodiment, the isolated antibody, or antigen binding portion thereof, binds to a portion and/or conformational epitope of the p40 subunit comprising at least one amino acid residue of loop 2 selected from the group consisting of residues 58-60, or within 1-10 Å of said amino acid residue.

In one embodiment, the isolated antibody, or antigen binding portion thereof, binds to a portion and/or conformational epitope of the p40 subunit comprising at least one amino acid residue of loop 3 selected from the group consisting of residues 84-94, or within 1-10 Å of said amino acid residue. In one embodiment, the isolated antibody, or antigen binding portion thereof, binds to a portion and/or conformational epitope of the p40 subunit comprising at least one amino acid residue of loop 3 selected from the group consisting of residues 85-93, or within 1-10 Å of said amino acid residue. In one embodiment, the isolated antibody, or antigen binding portion thereof, binds to a portion and/or conformational epitope of the p40 subunit comprising at least one amino acid residue of loop 3 selected from the group consisting of residues 86-89 and 93, or within 1-10 Å of said amino acid residue. In one embodiment, the isolated antibody, or antigen binding portion thereof, binds to a portion and/or conformational epitope of the p40 subunit comprising at least one amino acid residue of loop 3 selected from the group consisting of residues 86, 87, 89 and 93, or within 1-10 Å of said amino acid residue. In one embodiment, the isolated antibody, or antigen binding portion thereof, binds to a portion and/or conformational epitope of the p40 subunit comprising amino acid residue 87 of loop 3, or within 1-10 Å of said amino acid residue.

In one embodiment, the isolated antibody, or antigen binding portion thereof, binds to a portion and/or conformational epitope of the p40 subunit comprising at least one amino acid residue of loop 4 selected from the group consisting of residues 95-107, or within 1-10 Å of said amino acid residue. In one embodiment, the isolated antibody, or antigen binding portion thereof, binds to a portion and/or conformational epitope of the p40 subunit comprising at least one amino acid residue of loop 4 selected from the group consisting of residues 102-104, or within 1-10 Å of said amino acid residue.

In one embodiment, the isolated antibody, or antigen binding portion thereof, binds to a portion and/or conformational epitope of the p40 subunit comprising at least one amino acid residue of loop 5 selected from the group consisting of residues 124-129, or within 1-10 Å of said amino acid residue.

In one embodiment, the isolated antibody, or antigen binding portion thereof, binds to a portion and/or conformational epitope of the p40 subunit comprising at least one amino acid residue of loop 6 selected from the group consisting of residues 157-164, or within 1-10 Å of said amino acid residue.

In one embodiment, the isolated antibody, or antigen binding portion thereof, binds to a portion and/or conformational epitope of the p40 subunit comprising at least one amino acid residue of loop 7 selected from the group consisting of residues 194-197, or within 1-10 Å of said amino acid residue.

In one embodiment, the isolated antibody, or antigen binding portion thereof, binds to a portion and/or conformational epitope of the p40 subunit comprising at least one amino acid residue of loops 1-4 selected from the group consisting of residues 14-23, 58-60, 84-94 and 95-107, or within 1-10 Å of said amino acid residue. In one embodiment, the isolated antibody, or antigen binding portion thereof, binds to a portion and/or conformational epitope of the p40 subunit comprising at least one amino acid residue of loops 1-4 selected from the group consisting of residues 14-18, 85-93 and 102-104, or within 1-10 Å of said amino acid residue. In one embodiment, the isolated antibody, or antigen binding portion thereof, binds to a portion and/or conformational epitope of the p40 subunit comprising at least one amino acid residue of loops 1-4 selected from the group consisting of residues 14-17, 86-89, 93 and 103-104, or within 1-10 Å of said amino acid residue. In one embodiment, the isolated antibody, or antigen binding portion thereof, binds to a portion and/or conformational epitope of the p40 subunit comprising at least one amino acid residue of loops 1-4 selected from the group consisting of residues 15-17, 86-87, 89, 93 and 104, or within 1-10 Å of said amino acid residue.

In one embodiment, the isolated antibody, or antigen binding portion thereof, binds to a portion and/or conformational epitope of the p40 subunit comprising at least one amino acid residue of loops 1-2 selected from the group consisting of residues 14-23 and 58-60, or within 1-10 Å of said amino acid residue. In one embodiment, the isolated antibody, or antigen binding portion thereof, binds to a portion and/or conformational epitope of the p40 subunit comprising at least one amino acid residue of loops 1-2 selected from the group consisting of residues 15, 17-21, 23 and 58-60, or within 1-10 Å of said amino acid residue.

In one embodiment, the isolated antibody, or antigen binding portion thereof, binds to a portion and/or conformational epitope of the p40 subunit comprising at least one amino acid residue of loop 1 selected from the group consisting of residues 14-23 and at least one amino acid residue of loop 2 selected from the group consisting of residues 58-60, or within 1-10 Å of said amino acid residue.

In one embodiment, the isolated antibody, or antigen binding portion thereof, binds to a portion and/or conformational epitope of the p40 subunit comprising at least one amino acid residue of loops 1 and 3 selected from the group consisting of residues 14-23 and 84-94, or within 1-10 Å of said amino acid residue.

In one embodiment, the isolated antibody, or antigen binding portion thereof, binds to a portion and/or conformational epitope of the p40 subunit comprising at least one amino acid residue of loop 1 selected from the group consisting of residues 14-23 and at least one amino acid residue of loop 3 selected from the group consisting of residues 84-94, or within 1-10 Å of said amino acid residue.

In one embodiment, the isolated antibody, or antigen binding portion thereof, binds to a portion and/or conformational epitope of the p40 subunit comprising at least one amino acid residue of loops 1 and 4 selected from the group consisting of residues 14-23 and 95-107, or within 1-10 Å of said amino acid residue.

In one embodiment, the isolated antibody, or antigen binding portion thereof, binds to a portion and/or conformational epitope of the p40 subunit comprising at least one amino acid residue of loop 1 selected from the group consisting of residues 14-23 and at least one amino acid residue of loop 4 selected from the group consisting of residues 95-107, or within 1-10 Å of said amino acid residue.

In one embodiment, the isolated antibody, or antigen binding portion thereof, binds to a portion and/or conformational epitope of the p40 subunit comprising at least one amino acid residue of loops 3 and 4 selected from the group consisting of residues 84-94 and 95-107, or within 1-10 Å of said amino acid residue.

In one embodiment, the isolated antibody, or antigen binding portion thereof, binds to a portion and/or conformational epitope of the p40 subunit comprising at least one amino acid residue of loop 3 selected from the group consisting of residues 84-94 and at least one amino acid residue of loop 4 selected from the group consisting of residues 95-107, or within 1-10 Å of said amino acid residue.

In another embodiment, the invention provides an isolated antibody that competes for binding with any of the foregoing antibodies, or antigen binding portion thereof.

In yet another embodiment, the isolated antibody, or antigen binding portion thereof, is not the antibody Y61 or J695.

In another aspect, the invention provides an isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein any one of the variable region residues other than amino acid residues 27, 32, 52, 53, 97, 101 and 102 of SEQ ID NO: 1 and amino acid residues 35, 51 and 90-101 of SEQ ID NO: 2 are independently substituted with a different amino acid.

In another aspect, the invention provides an isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein one or more of the variable region amino acid residues 27, 32, 52, 53, 97, 101 and 102 of SEQ ID NO: 1 and 35, 51 and 90-101 of SEQ ID NO: 2 are independently substituted with a different amino acid residue.

In one embodiment, one or more of the variable region amino acid residues 27, 32 and 102 of SEQ ID NO: 1 are independently substituted with an aromatic residue.

In one embodiment, one or more of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln.

In one embodiment, one or more of the variable region amino acid residues 92 and 97 of SEQ ID NO: 2 are independently substituted with an aromatic amino acid residue.

In one embodiment, one or more of the variable region amino acid residues 101 of SEQ ID NO: 1 and 51 of SEQ ID NO: 2 are independently substituted with an amino acid residue selected from the group consisting of Tyr, Ser, Thr, Asn and Gln.

In one embodiment, the variable region amino acid residue 91 of SEQ ID NO: 2 is substituted with any amino acid residue except Gln.

In one embodiment, the variable region amino acid residue 95 of SEQ ID NO: 2 is substituted with a different aromatic amino acid residue.

In one embodiment, the variable region amino acid residue 97 of SEQ ID NO: 2 is substituted with an amino acid residue selected from the group consisting of Phe, Tyr, Trp, His, Asp, Glu, Asn and Gln.

In one embodiment, one or more of the variable region amino acid residues 90-101 of SEQ ID NO: 2 is independently substituted with at least one or more different amino acids, and wherein the length of CDRL3 of the antibody is greater than or equal to 12 amino acid residues.

In one embodiment, the isolated antibody has one or more of the following substitutions: (a) one or more of the variable region amino acid residues 90-101 of SEQ ID NO: 2 is independently substituted with at least one or more different amino acids, and wherein the length of CDRL3 of the antibody is greater than or equal to 12 amino acid residues; (b) variable region amino acid residue 91 of SEQ ID NO: 2 is substituted with any amino acid residue except Gln; (c) variable region amino acid residue 95 of SEQ ID NO: 2 is substituted with a different aromatic amino acid residue; or (d) variable region amino acid residue 97 of SEQ ID NO: 2 is substituted with an amino acid residue selected from the group consisting of Phe, Tyr, Trp, His, Asp, Glu, Asn and Gln.

In one embodiment, one or more of the variable region amino acid residues 52 and 53 of SEQ ID NO: 1 is independently substituted with an amino acid residue selected from the group consisting of Tyr, Ser, Thr, Asn, Gln, Lys and Arg.

In another aspect, the invention provides an isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein one or more of the variable region amino acid residues 33, 50, 57 and 99 of SEQ ID NO: 1 and 33 of SEQ ID NO: 2 are independently substituted with a different amino acid residue.

In one embodiment, variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys. In one embodiment, variable region amino acid residue 33 of SEQ ID NO: 1 is substituted with Lys.

In one embodiment, variable region amino acid residue 50 of SEQ ID NO: 1 is substituted with an amino acid residue selected from the group consisting of Phe, Tyr, Trp, His, Met, Gln, Arg and Lys. In one embodiment, variable region amino acid residue 50 of SEQ ID NO: 1 is substituted with Tyr or Trp.

In one embodiment, variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln. In one embodiment, variable region amino acid residue 57 of SEQ ID NO: 1 is substituted with Ile or Trp. In one embodiment, variable region amino acid residue 57 of SEQ ID NO: 1 is substituted with Ser or Thr.

In one embodiment, variable region amino acid residue 99 of SEQ ID NO: 1 is substituted with an amino acid residue selected from the group consisting of Phe, Tyr, Trp, His, Met, Arg and Lys. In one embodiment, variable region amino acid residue 99 of SEQ ID NO: 1 is substituted with Tyr or Trp.

In one embodiment, variable region amino acid residue 33 of SEQ ID NO: 2 is substituted with an amino acid residue selected from the group consisting of Phe, Tyr, Trp, His, Gln and Lys. In one embodiment, variable region amino acid residue 33 of SEQ ID NO: 2 is substituted with Tyr or Trp.

In one embodiment, the isolated antibody, or antigen binding portion thereof, is not the antibody J695 or Y61.

In another aspect, the invention provides an isolated antibody that competes for binding with any of the foregoing antibodies, or antigen binding portion thereof.

In yet another aspect, the invention provides a method for altering the activity of an isolated antibody that binds to the p40 subunit of IL-12 and/or IL-23, or antigen binding portion thereof, wherein said antibody or antigen binding portion thereof comprises the heavy chain variable region amino acid sequence of SEQ ID NO: 1 and the light chain variable region amino acid sequence of SEQ ID NO: 2, comprising independently substituting one or more of the variable region amino acid residues 27, 32, 52, 53, 97, 101 and 102 of SEQ ID NO: 1 and amino acid residues 35, 51 and 90-101 of SEQ ID NO: 2 with a different amino acid residue, thereby altering the activity of an antibody that binds to the p40 subunit of IL-12 and/or IL-23, or antigen binding portion thereof.

In one embodiment, one or more of the variable region amino acid residues 27, 32 and 102 of SEQ ID NO: 1 are independently substituted with an aromatic residue.

In one embodiment, one or more of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln.

In one embodiment, one or more of the variable region amino acid residues 92 and 97 of SEQ ID NO: 2 are independently substituted with an aromatic amino acid residue.

In one embodiment, one or more of the variable region amino acid residues 101 of SEQ ID NO: 1 and 51 of SEQ ID NO: 2 are independently substituted with an amino acid residue selected from the group consisting of Tyr, Ser, Thr, Asn and Gln.

In one embodiment, the variable region amino acid residue 91 of SEQ ID NO: 2 is substituted with any amino acid residue except Gln.

In one embodiment, the variable region amino acid residue 95 of SEQ ID NO: 2 is substituted with a different aromatic amino acid residue.

In one embodiment, the variable region amino acid residue 97 of SEQ ID NO: 2 is substituted with an amino acid residue selected from the group consisting of Phe, Tyr, Trp, His, Asp, Glu, Asn and Gln.

In one embodiment, one or more of the variable region amino acid residues 90-101 of SEQ ID NO: 2 are independently substituted with at least one or more different amino acids, and wherein the length of CDRL3 of the antibody is greater than or equal to 12 amino acid residues.

In one embodiment, the isolated antibody, or antigen binding portion thereof, has one or more of the following substitutions: (a) one or more of the variable region amino acid residues 90-101 of SEQ ID NO: 2 are independently substituted with at least one or more different amino acids, and wherein the length of CDRL3 of the antibody is greater than or equal to 12 amino acid residues; (b) variable region amino acid residue 91 of SEQ ID NO: 2 is substituted with any amino acid residue except Gln; (c) variable region amino acid residue 95 of SEQ ID NO: 2 is substituted with a different aromatic amino acid residue; or (d) variable region amino acid residue 97 of SEQ ID NO: 2 is substituted with an amino acid residue selected from the group consisting of Phe, Tyr, Trp, His, Asp, Glu, Asn and Gln.

In one embodiment, one or more of the variable region amino acid residues 52 and 53 of SEQ ID NO: 1 are independently substituted with an amino acid residue selected from the group consisting of Tyr, Ser, Thr, Asn, Gln, Lys and Arg.

In one embodiment, an isolated antibody, or antigen binding portion thereof, of the invention further binds to one or more of the epitopes described in US 2009/0202549, the entire contents of which are hereby incorporated by reference herein.

In another aspect, the invention provides a method for altering the activity of an isolated antibody that binds to the p40 subunit of IL-12 and/or IL-23, or antigen binding portion thereof, wherein said antibody or antigen binding portion thereof comprises the heavy chain variable region amino acid sequence of SEQ ID NO: 1 and the light chain variable region amino acid sequence of SEQ ID NO: 2, comprising independently substituting one or more of the variable region amino acid residues 33, 50, 57 and 99 of SEQ ID NO: 1 and 33 of SEQ ID NO: 2 with a different amino acid residue, thereby altering the activity of an antibody that binds to the p40 subunit of IL-12 and/or IL-23, or antigen binding portion thereof.

In one embodiment, variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys. In one embodiment, variable region amino acid residue 33 of SEQ ID NO: 1 is substituted with Lys.

In one embodiment, variable region amino acid residue 50 of SEQ ID NO: 1 is substituted with an amino acid residue selected from the group consisting of Phe, Tyr, Trp, His, Met, Gln, Arg and Lys. In one embodiment, variable region amino acid residue 50 of SEQ ID NO: 1 is substituted with Tyr or Trp.

In one embodiment, variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln. In one embodiment, variable region amino acid residue 57 of SEQ ID NO: 1 is substituted with Ile or Trp. In one embodiment, variable region amino acid residue 57 of SEQ ID NO: 1 is substituted with Ser or Thr.

In one embodiment, variable region amino acid residue 99 of SEQ ID NO: 1 is substituted with an amino acid residue selected from the group consisting of Phe, Tyr, Trp, His, Met, Arg and Lys. In one embodiment, variable region amino acid residue 99 of SEQ ID NO: 1 is substituted with Tyr or Trp.

In one embodiment, variable region amino acid residue 33 of SEQ ID NO: 2 is substituted with an amino acid residue selected from the group consisting of Phe, Tyr, Trp, His, Gln and Lys. In one embodiment, variable region amino acid residue 33 of SEQ ID NO: 2 is substituted with Tyr or Trp.

In another embodiment, the invention provides an isolated antibody, or antigen binding portion thereof, produced according to the methods of the invention.

In a still further aspect, the invention provides an isolated antibody that binds to the p40 subunit of IL-12 and/or IL-23, or antigen binding portion thereof, wherein said antibody binds within 10 Å to a conformational epitope comprising at least one amino acid residue selected from the group consisting of amino acid residues 16, 87 and 93 of the amino acid sequence of SEQ ID NO:3. In one embodiment the isolated antibody, or antigen binding portion thereof, binds to amino acid residue 16.

In one embodiment, the isolated antibody, or antigen binding portion thereof, binds to the p40 subunit of IL-12 and/or IL-23 with a K_{off} of 1×10⁻³ or less or a K_{d} of 1×10⁻¹⁰ M or less.

In one embodiment, the isolated antibody, or antigen binding portion thereof, neutralizes the biological activity of the p40 subunit of Il-12 and/or IL-23.

In another aspect, the invention provides a pharmaceutical composition comprising an isolated antibody, or antigen binding portion thereof, of the invention and a pharmaceutical acceptable carrier or excipients. In one embodiment, the pharmaceutical composition further includes at least one additional biologically active agent.

In another aspect, the invention provides an isolated nucleic acid that encodes an antibody, or antigen binding portion thereof, of the invention.

In another aspect, the invention provides an isolated nucleic acid vector comprising a nucleic acid of the invention operably linked with at least one transcription regulatory nucleic acid sequence.

In still another aspect, the invention provides a host cell comprising a nucleic acid vector of the invention. In one embodiment, the host cell is a eukaryotic host cell or prokaryotic host cell.

In yet another aspect, the invention provides a method for diagnosing at least one IL-12 and/or IL-23 related condition in a subject. The method includes contacting a biological sample from the subject with an isolated antibody, or antigen binding portion thereof, of the invention, and measuring the amount of p40 subunit of IL-12 and/or IL-23 that is present in the sample, wherein the detection of elevated or reduced levels of the p40 subunit of IL-12 and/or IL-23 in the sample, as compared to a normal or control, is indicative of the presence or absence of an IL-12 and/or IL-23 related condition, thereby diagnosing at least one IL-12 and/or IL-23 related condition in the subject.

In one embodiment, the isolated antibody or antigen binding portion thereof contains a detectable label or is detected by a second molecule having a detectable label.

In another aspect, the invention provides a method for identifying an agent that modulates at least one of the expression, level, and/or activity of IL-12 and/or IL-23 in a biological sample. The method includes contacting the sample with an isolated antibody, or antigen binding portion thereof, of the invention and detecting the expression, level, and/or activity of IL-12 and/or IL-23 in the sample, wherein an increase or decrease in at least one of the expression, level, and/or activity of IL-12 and/or IL-23 compared to an untreated sample is indicative of an agent capable of modulating at least one of the expression, level, and/or activity of IL-12 and/or IL-23, thereby identifying an agent that modulates at least one of the expression, level and/or activity of IL-12 and/or IL-23 in the sample.

In one embodiment, the isolated antibody or antigen binding portion thereof contains a detectable label or is detectable by a second molecule having a detectable label.

In one embodiment, the invention provides an isolated antibody that binds to the p40 subunit of IL-12 and/or IL-23, or an antigen binding portion thereof, wherein said antibody binds to a portion of the p40 subunit comprising at least one, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196 or 197 amino acid residues selected from residues 1-197 of the amino acid sequence of SEQ ID NO: 3, or within 1-10 Å of said amino acid residue. In one embodiment, the antibody, or antigen-binding portion thereof, binds to a portion of the p40 subunit comprising residues 1-197 of the amino acid sequence of SEQ ID NO: 3.

In another embodiment, the invention provides an isolated antibody, or antigen binding portion thereof, wherein said antibody binds to a portion of the p40 subunit comprising at least one amino acid residue or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, or 107 amino acid residues selected from residues 1-107 of the amino acid sequence of SEQ ID NO: 3, or within 1-10 Å of said amino acid residue. In one embodiment, the antibody, or antigen-binding portion thereof, binds to a portion of the p40 subunit comprising residues 1-107 of the amino acid sequence of SEQ ID NO: 3.

In another embodiment, the invention provides an isolated antibody, or antigen binding portion thereof, wherein said antibody binds to a portion of the p40 subunit comprising at least one amino acid residue or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 amino acid residues of loops 1-7 of the p40 subunit, wherein the at least one amino acid residue or at least 2, 3, 4 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55 amino acid residues are selected from the group consisting of residues 14-23, 58-60, 84-107, 124-129, 157-164 and 194-197 of the amino acid sequence of SEQ ID NO: 3, or within 1-10 Å of said amino acid residue. In another embodiment, the antibody, or antigen binding portion thereof, binds to a portion of the p40 subunit comprising at least residues 14-23, 58-60, 84-107, 124-129, 157-164 and 194-197 of the amino acid sequence of SEQ ID NO: 3. In one embodiment, the antibody, or antigen-binding portion thereof, binds to a portion of the p40 subunit comprising residues 14-23, 58-60, 84-107, 124-129, 157-164 and 194-197 of the amino acid sequence of SEQ ID NO:3.

In another embodiment, the invention provides an isolated antibody, or antigen binding portion thereof, wherein said antibody binds to a portion of the p40 subunit comprising at least one amino acid residue, or at least 2, 3, 4, 5, 6, 7, 8, 9 or 10 amino acid residues, of loop 1 selected from the group consisting of residues 14-23, or within 1-10 Å of said amino acid residue. In one embodiment, the antibody, or antigen-binding portion thereof, binds to a portion of the p40 subunit comprising residues 14-23 of loop 1.

In one embodiment, the isolated antibody binds to a portion of the p40 subunit comprising at least one amino acid residue or at least two, at least three, at least four, or at least five amino acid residues of loop 1 selected from the group consisting of residues 14-18, or within 1-10 Å of said amino acid residue. In another embodiment, the antibody, or antigen-binding portion thereof, binds to a portion of the p40 subunit comprising residues 14-18 of loop 1.

In another embodiment, the isolated antibody binds to a portion of the p40 subunit comprising at least one amino acid residue, at least two, at least three, or at least four amino acid residues of loop 1 selected from the group consisting of residues 14-17, or within 1-10 Å of said amino acid residue. In another embodiment, the antibody, or antigen-binding portion thereof, binds to a portion of the p40 subunit comprising residues 14-17 of loop 1.

In yet another embodiment, the isolated antibody binds to a portion of the p40 subunit comprising at least one amino acid residue, at least two, or at least three amino acid residues of loop 1 selected from the group consisting of residues 15-17, or within 1-10 Å of said amino acid residue. In another embodiment, the antibody, or antigen-binding portion thereof, binds to a portion of the p40 subunit comprising residues 15-17 of loop 1.

In another embodiment, the isolated antibody binds to a portion of the p40 subunit comprising at least one amino acid residue, at least two amino acid residues, or at least three amino acid residues of loop 2 selected from the group consisting of residues 58-60, or within 1-10 Å of said amino acid residue. In another embodiment, the antibody, or antigen-binding portion thereof, binds to a portion of the p40 subunit comprising residues 58-60 of loop 2.

In another embodiment, the isolated antibody or antigen binding portion thereof, binds to a portion of the p40 subunit comprising at least one amino acid residue, or at least 2, 3, 4, 5, 6, 7, 8, 9 or 10 amino acid residues of loop 3 selected from the group consisting of residues 84-94, or within 1-10 Å of said amino acid residue. In another embodiment, the antibody, or antigen-binding portion thereof, binds to a portion of the p40 subunit comprising residues 84-94 of loop 3.

In another embodiment, the isolated antibody, or antigen-binding portion thereof, binds to a portion of the p40 subunit comprising at least one amino acid residue or at least 2, 3, 4, 5, 6, 7, 8 or 9 amino acid residues of loop 3 selected from the group consisting of residues 85-93, or within 1-10 Å of said amino acid residue. In another embodiment, the antibody, or antigen-binding portion thereof, binds to a portion of the p40 subunit comprising residues 85-93 of loop 3.

In another embodiment, the isolated antibody, or antigen-binding portion thereof, binds to a portion of the p40 subunit comprising at least one amino acid residue, at least two, three, four or five amino acid residues of loop 3 selected from the group consisting of residues 86-89 and 93, or within 1-10 Å of said amino acid residue. In another embodiment, the antibody, or antigen-binding portion thereof, binds to a portion of the p40 subunit comprising residues 86-89 and 93 of loop 3.

In another embodiment, the isolated antibody binds to a portion of the p40 subunit comprising at least one amino acid residue, at least two, three or four amino acid residues of loop 3 selected from the group consisting of residues 86, 87, 89 and 93, or within 1-10 Å of said amino acid residue. In another embodiment, the antibody, or antigen-binding portion thereof, binds to a portion of the p40 subunit comprising residues 86, 87, 89 and 93 of loop 3.

In yet another embodiment, the isolated antibody binds to a portion of the p40 subunit comprising amino acid residue 87 of loop 3, or within 1-10 Å of said amino acid residue.

In another embodiment, the isolated antibody, or antigen binding portion thereof, binds to a portion of the p40 subunit comprising at least one amino acid, at least 2, 3, 4, 5, 6, 7, 8, 9, 10, 11, 12 or 13 amino acid residues of loop 4 selected from the group consisting of residues 95-107, or within 1-10 Å of said amino acid residue. In another embodiment, the antibody, or antigen-binding portion thereof, binds to a portion of the p40 subunit comprising residues 95-107 of loop 4.

In another embodiment, the isolated antibody, or antigen-binding portion thereof, binds to a portion of the p40 subunit comprising at least one, two or three amino acid residues of loop 4 selected from the group consisting of residues 102-104, or within 1-10 Å of said amino acid residue. In another embodiment, the antibody, or antigen-binding portion thereof, binds to a portion of the p40 subunit comprising residues 102-104 of loop 4.

In another embodiment, the isolated antibody, or antigen-binding portion thereof, binds to a portion of the p40 subunit comprising at least one amino acid residue, or at least 2, 3, 4, 5 or 6 amino acid residues of loop 5 selected from the group consisting of residues 124-129, or within 1-10 Å of said amino acid residue. In another embodiment, the antibody, or antigen-binding portion thereof, binds to a portion of the p40 subunit comprising residues 124-129 of loop 5.

In another embodiment, the isolated antibody, or antigen-binding portion thereof, binds to a portion of the p40 subunit comprising at least one amino acid residue or at least 2, 3, 4, 5, 6, 7 or 8 amino acid residues of loop 6 selected from the group consisting of residues 157-164, or within 1-10 Å of said amino acid residue. In another embodiment, the antibody, or antigen-binding portion thereof, binds to a portion of the p40 subunit comprising residues 157-164 of loop 6.

In another embodiment, the isolated antibody, or antigen-binding portion thereof, binds to a portion of the p40 subunit comprising at least one amino acid residue or at least 2, 3 or 4 amino acid residues of loop 7 selected from the group consisting of residues 194-197, or within 1-10 Å of said amino acid residue. In another embodiment, the antibody, or antigen-binding portion thereof, binds to a portion of the p40 subunit comprising residues 194-197 of loop 7.

In another embodiment, the isolated antibody, or antigen binding portion thereof, binds to a portion of the p40 subunit comprising at least one amino acid residue or at least 2, 3, 4 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 amino acid residues of loops 1-4 selected from the group consisting of residues 14-23, 58-60, 84-94 and 95-107, or within 1-10 Å of said amino acid residue. In another embodiment, the antibody, or antigen-binding portion thereof, binds to a portion of the p40 subunit comprising residues 14-23, 58-60, 84-94 and 95-107 of loops 1-4.

In another embodiment, the invention provides an isolated antibody, or antigen-binding portion thereof, that binds to a portion of the p40 subunit comprising at least one amino acid residue or at least 2, 3, 4, 5, 6, 7, 8, 9, 10, 11, 12, 13, 14, 15, 16 or 17 amino acid residues of loops 1-4 selected from the group consisting of residues 14-18, 85-93 and 102-104, or within 1-10 Å of said amino acid residue. In another embodiment, the antibody, or antigen-binding portion thereof, binds to a portion of the p40 subunit comprising residues 14-18, 85-93 and 102-104 of loops 1-4.

In another embodiment, the isolated antibody, or antigen-binding portion thereof, binds to a portion of the p40 subunit comprising at least one amino acid residue or at least 2, 3, 4, 5, 6, 7, 8, 9, 10 or 11 amino acid residues of loops 1-4 selected from the group consisting of residues 14-17, 86-89, 93 and 103-104, or within 1-10 Å of said amino acid residue. In another embodiment, the antibody, or antigen-binding portion thereof, binds to a portion of the p40 subunit comprising residues 14-17, 86-89, 93 and 103-104 of loops 1-4.

In another embodiment, the isolated antibody or antigen-binding portion thereof binds to a portion of the p40 subunit comprising at least one amino acid residue, at least 2, 3, 4, 5, 6, 7, or 8 amino acid residues of loops 1-4 selected from the group consisting of residues 15-17, 86-87, 89, 93 and 104, or within 1-10 Å of said amino acid residue. In another embodiment, the antibody, or antigen-binding portion thereof, binds to a portion of the p40 subunit comprising residues 15-17, 86-87, 89, 93 and 104 of loops 1-4.

In another embodiment, the isolated antibody, or antigen binding portion thereof, binds to a portion of the p40 subunit comprising at least one amino acid residue, at least 2, 3, 4, 5, 6, 7, 8, 9, 10, 11, 12, or 13 amino acid residues of loops 1-2 selected from the group consisting of residues 14-23 and 58-60, or within 1-10 Å of said amino acid residue. In another embodiment, the antibody, or antigen-binding portion thereof, binds to a portion of the p40 subunit comprising residues 14-23 and 58-60 of loops 1-2.

In another embodiment, the isolated antibody, or antigen binding portion thereof, binds to a portion of the p40 subunit comprising at least one amino acid residue or at least 2, 3, 4, 5, 6, 7, 8, 9 or 10 amino acid residues of loops 1-2 selected from the group consisting of residues 15, 17-21, 23 and 58-60, or within 1-10 Å of said amino acid residue. In another embodiment, the antibody, or antigen-binding portion thereof, binds to a portion of the p40 subunit comprising residues 15, 17-21, 23 and 58-60 of loops 1-2.

In another embodiment, the isolated antibody, or antigen-binding portion thereof, binds to a portion of the p40 subunit comprising at least one amino acid residue or at least 2, 3, 4, 5, 6, 7, 8, 9 or 10 amino acid residues of loop 1 selected from the group consisting of residues 14-23 and at least one amino acid residue or at least 2 or 3 amino acid residues of loop 2 selected from the group consisting of residues 58-60, or within 1-10 Å of said amino acid residue. In another embodiment, the antibody, or antigen-binding portion thereof, binds to a portion of the p40 subunit comprising residues 14-23 of loop 1 and residues 58-60 of loop 2.

In another embodiment, the isolated antibody, or antigen-binding portion thereof, binds to a portion of the p40 subunit comprising at least one amino acid residue or at least 2, 3, 4, 5, 6, 7, 8, 9, 10, 11, 12, 13, 14, 15, 16, 17, 18, 19, 20 or 21 amino acid residues of loops 1 and 3 selected from the group consisting of residues 14-23 and 84-94, or within 1-10 Å of said amino acid residue. In another embodiment, the antibody, or antigen-binding portion thereof, binds to a portion of the p40 subunit comprising residues 14-23 and 84-94 of loops 1 and 3.

In another embodiment, the isolated antibody, or antigen-binding portion thereof, binds to a portion of the p40 subunit comprising at least one amino acid residue or at least 2, 3, 4, 5, 6, 7, 8, 9 or 10 amino acid residues of loop 1 selected from the group consisting of residues 14-23 and at least one amino acid residue or at least 2, 3, 4, 5, 6, 7, 8, 9, 10 or 11 amino acid residues of loop 3 selected from the group consisting of residues 84-94, or within 1-10 Å of said amino acid residue. In another embodiment, the antibody, or antigen-binding portion thereof, binds to a portion of the p40 subunit comprising residues 14-23 of loop 1 and residues 84-94 of loop 3.

In another embodiment, the isolated antibody, or antigen binding portion thereof, binds to a portion of the p40 subunit comprising at least one amino acid residue, or at least 2, 3, 4, 5, 6, 7, 8, 9, 10, 11, 12, 13, 14, 15, 16, 17, 18, 19, 20, 21, 22 or 23 amino acid residues of loops 1 and 4 selected from the group consisting of residues 14-23 and 95-107, or within 1-10 Å of said amino acid residue. In another embodiment, the antibody, or antigen-binding portion thereof, binds to a portion of the p40 subunit comprising residues 14-23 and 95-107 of loops 1 and 4.

In another embodiment, the isolated antibody, or antigen-binding portion thereof, binds to a portion of the p40 subunit comprising at least one amino acid residue, at least 2, 3, 4, 5, 6, 7, 8, 9 or 10 amino acid residues of loop 1 selected from the group consisting of residues 14-23 and at least one amino acid residue or at least 2, 3, 4, 5, 6, 7, 8, 9, 10, 11, 12, or 13 amino acid residues of loop 4 selected from the group consisting of residues 95-107, or within 1-10 Å of said amino acid residue. In another embodiment, the antibody, or antigen-binding portion thereof, binds to a portion of the p40 subunit comprising residues 14-23 of loop 2 and 95-107 of loop 4.

In another embodiment, the isolated antibody, or antigen-binding portion thereof, binds to a portion of the p40 subunit comprising at least one amino acid residue or at least 2, 3, 4, 5, 6, 7, 8, 9, 10, 11, 12, 13, 14, 15, 16, 17, 18, 19, 20, 21, 22, 23 or 24 amino acid residues of loops 3 and 4 selected from the group consisting of residues 84-94 and 95-107, or within 1-10 Å of said amino acid residue. In another embodiment, the antibody, or antigen-binding portion thereof, binds to a portion of the p40 subunit comprising residues 84-94 and 95-107 of loops 3 and 4.

In another embodiment, the isolated antibody, or antigen-binding portion thereof, binds to a portion of the p40 subunit comprising at least one amino acid residue or at least 2, 3, 4, 5, 6, 7, 8, 9, 10 or 11 amino acid residues of loop 3 selected from the group consisting of residues 84-94 and at least one amino acid residue or at least 2, 3, 4, 5, 6, 7, 8, 9, 10, 11, 12 or 13 amino acid residues of loop 4 selected from the group consisting of residues 95-107, or within 1-10 Å of said amino acid residue. In another embodiment, the antibody, or antigen-binding portion thereof, binds to a portion of the p40 subunit comprising residues 84-94 of loop 3 and residues 95-107 of loop 4.

In another embodiment, the invention provides an isolated antibody, or antigen-binding portion thereof, that competes for binding with any antibody, or antigen binding portion thereof, disclosed herein.

In one embodiment, the isolated antibody, or antigen-binding portion thereof, is not the antibody Y61 or J695.

In one embodiment, the invention provides an isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein any one of the variable region residues other than amino acid residues 27, 32, 52, 53, 97, 101 and 102 of SEQ ID NO: 1 and amino acid residues 35, 51 and 90, 91, 92, 93, 94, 95, 96, 97, 98, 99, 100 and 101 of SEQ ID NO: 2 are independently substituted with a different amino acid. In one embodiment, at least 2, 3, 4, 5, 6, 7, 8, 9, 10, 11, 12, 13, 14, 15, 16, 17, 18, 19, 20, 21, 22, 23, 24, 25, 30, 35, 40, 45, 50 or more of the variable region residues other than amino acid residues 27, 32, 52, 53, 97, 101 and 102 of SEQ ID NO: 1 and amino acid residues 35, 51 and 90, 91, 92, 93, 94, 95, 96, 97, 98, 99, 100 and 101 of SEQ ID NO: 2 are independently substituted with a different amino acid.

In another embodiment, the invention provides an isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein one or more of the variable region amino acid residues 27, 32, 52, 53, 97, 101 and 102 of SEQ ID NO: 1 and 35, 51 and 90, 91, 92, 93, 94, 95, 96, 97, 98, 99, 100 and 101 of SEQ ID NO: 2 are independently substituted with a different amino acid residue. In another embodiment, at least 2, 3, 4, 5, 6, 7, 8, 9, 10, 11, 12, 13, 14, 15, 16, 17, 18, 19, 20 or 21 of the variable region amino acid residues 27, 32, 52, 53, 97, 101 and 102 of SEQ ID NO: 1 and 35, 51 and 90, 91, 92, 93, 94, 95, 96, 97, 98, 99, 100 and 101 of SEQ ID NO: 2 are independently substituted with a different amino acid residue. In one embodiment, variable region amino acid residues 27, 32, 52, 53, 97, 101 and 102 of SEQ ID NO: 1 and 35, 51 and 90, 91, 92, 93, 94, 95, 96, 97, 98, 99, 100 and 101 of SEQ ID NO: 2 are independently substituted with a different amino acid residue.

In one embodiment, one, two or three of the variable region amino acid residues 27, 32 and 102 of SEQ ID NO: 1 are independently substituted with an aromatic residue. In another embodiment, variable region amino acid residues 27, 32 and 102 of SEQ ID NO: 1 are independently substituted with an aromatic residue.

In another embodiment, one, two or three of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln. In another embodiment, the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln.

In another embodiment, one or two of the variable region amino acid residues 92 and 97 of SEQ ID NO: 2 are independently substituted with an aromatic amino acid residue. In another embodiment, the variable region amino acid residues 92 and 97 of SEQ ID NO: 2 are independently substituted with an aromatic amino acid residue.

In another embodiment, one or two of the variable region amino acid residues 101 of SEQ ID NO: 1 and 51 of SEQ ID NO: 2 are independently substituted with an amino acid residue selected from the group consisting of Tyr, Ser, Thr, Asn and Gln. In another embodiment, the variable region amino acid residues 101 of SEQ ID NO: 1 and 51 of SEQ ID NO: 2 are independently substituted with an amino acid residue selected from the group consisting of Tyr, Ser, Thr, Asn and Gln.

In another embodiment, the variable region amino acid residue 91 of SEQ ID NO: 2 is substituted with any amino acid residue except Gln. In yet another embodiment, the variable region amino acid residue 95 of SEQ ID NO: 2 is substituted with a different aromatic amino acid residue. In another embodiment, the variable region amino acid residue 97 of SEQ ID NO: 2 is substituted with an amino acid residue selected from the group consisting of Phe, Tyr, Trp, His, Asp, Glu, Asn and Gln.

In another embodiment, at least one, 2, 3, 4, 5, 6, 7, 8, 9, 10, 11 or 12 of the variable region amino acid residues 90-101 of SEQ ID NO: 2 is independently substituted with at least one or more different amino acids, and wherein the length of CDRL3 of the antibody is greater than or equal to 12 amino acid residues.

In another embodiment, the antibody, or antigen-binding portion thereof, has one or more of the following substitutions: (a) one, 2, 3, 4, 5, 6, 7, 8, 9, 10, 11 or 12 of the variable region amino acid residues 90-101 of SEQ ID NO: 2 is independently substituted with at least one or more different amino acids, and wherein the length of CDRL3 of the antibody is greater than or equal to 12 amino acid residues; (b) variable region amino acid residue 91 of SEQ ID NO: 2 is substituted with any amino acid residue except Gln; (c) variable region amino acid residue 95 of SEQ ID NO: 2 is substituted with a different aromatic amino acid residue; or (d) variable region amino acid residue 97 of SEQ ID NO: 2 is substituted with an amino acid residue selected from the group consisting of Phe, Tyr, Trp, His, Asp, Glu, Asn and Gln. In another embodiment, all of the variable region amino acid residues 90-101 of SEQ ID NO: 2 is independently substituted with at least one or more different amino acids, and wherein the length of CDRL3 of the antibody is greater than or equal to 12 amino acid residues.

In another embodiment, one or two of the variable region amino acid residues 52 and 53 of SEQ ID NO: 1 is independently substituted with an amino acid residue selected from the group consisting of Tyr, Ser, Thr, Asn, Gln, Lys and Arg. In one embodiment, the variable region amino acid residues 52 and 53 of SEQ ID NO: 1 is independently substituted with an amino acid residue selected from the group consisting of Tyr, Ser, Thr, Asn, Gln, Lys and Arg.

In another embodiment, the invention provides an isolated antibody, or antigen-binding portion thereof, that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein one, 2, 3, 4 or 5 of the variable region amino acid residues 33, 50, 57 and 99 of SEQ ID NO: 1 and 33 of SEQ ID NO: 2 are independently substituted with a different amino acid residue. In another embodiment, the variable region amino acid residues 33, 50, 57 and 99 of SEQ ID NO: 1 and 33 of SEQ ID NO: 2 are independently substituted with a different amino acid residue.

In one embodiment, variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys. In another embodiment, variable region amino acid residue 50 of SEQ ID NO: 1 is substituted with an amino acid residue selected from the group consisting of Phe, Tyr, Trp, His, Met, Gln, Arg and Lys. In another embodiment, variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln. In another embodiment, variable region amino acid residue 99 of SEQ ID NO: 1 is substituted with an amino acid residue selected from the group consisting of Phe, Tyr, Trp, His, Met, Arg and Lys. In another embodiment, variable region amino acid residue 33 of SEQ ID NO: 2 is substituted with an amino acid residue selected from the group consisting of Phe, Tyr, Trp, His, Gln and Lys.

In another embodiment, variable region amino acid residue 33 of SEQ ID NO: 1 is substituted with Lys. In another embodiment, variable region amino acid residue 50 of SEQ ID NO: 1 is substituted with Tyr or Trp. In another embodiment, variable region amino acid residue 57 of SEQ ID NO: 1 is substituted with Ile or Trp. In another embodiment, variable region amino acid residue 57 of SEQ ID NO: 1 is substituted with Ser or Thr. In another embodiment, variable region amino acid residue 99 of SEQ ID NO: 1 is substituted with Tyr or Trp. In another embodiment, variable region amino acid residue 33 of SEQ ID NO: 2 is substituted with Tyr or Trp.

In one embodiment, the isolated antibody, or antigen-binding portion thereof, is not the antibody J695 or Y61.

In another embodiment, the invention provides an isolated antibody, or antigen-binding portion thereof, that competes for binding with any of the antibodies or antigen-binding portions thereof disclosed herein.

In one embodiment, the invention provides a method for altering the activity of an antibody that binds to the p40 subunit of IL-12 and/or IL-23, or antigen binding portion thereof, wherein said antibody or antigen binding portion thereof comprises the heavy chain variable region amino acid sequence of SEQ ID NO: 1 and the light chain variable region amino acid sequence of SEQ ID NO: 2, comprising independently substituting at least one, 2, 3, 4, 5, 6, 7, 8, 9, 10, 11, 12, 13, 14, 15, 16, 17, 18, 19, 20 or 21 of the variable region amino acid residues 27, 32, 52, 53, 97, 101 and 102 of SEQ ID NO: 1 and amino acid residues 35, 51 and 90-101 of SEQ ID NO: 2 with a different amino acid residue, thereby altering the activity of an antibody that binds to the p40 subunit of IL-12 and/or IL-23, or antigen binding portion thereof. In one embodiment, the variable region amino acid residues 27, 32, 52, 53, 97, 101 and 102 of SEQ ID NO: 1 and amino acid residues 35, 51 and 90-101 of SEQ ID NO: 2 are substituted with a different amino acid residue, thereby altering the activity of an antibody that binds to the p40 subunit of IL-12 and/or IL-23, or antigen-binding portion thereof.

In one embodiment, one, two or three of the variable region amino acid residues 27, 32 and 102 of SEQ ID NO: 1 are independently substituted with an aromatic residue. In another embodiment, the variable region amino acid residues 27, 32 and 102 of SEQ ID NO: 1 are independently substituted with an aromatic residue. In another embodiment, one or two of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln. In another embodiment, the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln. In another embodiment, one or two of the variable region amino acid residues 92 and 97 of SEQ ID NO: 2 are independently substituted with an aromatic amino acid residue. In another embodiment, the variable region amino acid residues 92 and 97 of SEQ ID NO: 2 are independently substituted with an aromatic amino acid residue. In another embodiment, one or two of the variable region amino acid residues 101 of SEQ ID NO: 1 and 51 of SEQ ID NO: 2 are independently substituted with an amino acid residue selected from the group consisting of Tyr, Ser, Thr, Asn and Gln. In another embodiment, the variable region amino acid residues 101 of SEQ ID NO: 1 and 51 of SEQ ID NO: 2 are independently substituted with an amino acid residue selected from the group consisting of Tyr, Ser, Thr, Asn and Gln.

In another embodiment, the variable region amino acid residue 91 of SEQ ID NO: 2 is substituted with any amino acid residue except Gln. In another embodiment, the variable region amino acid residue 95 of SEQ ID NO: 2 is substituted with a different aromatic amino acid residue. In another embodiment, the variable region amino acid residue 97 of SEQ ID NO: 2 is substituted with an amino acid residue selected from the group consisting of Phe, Tyr, Trp, His, Asp, Glu, Asn and Gln. In another embodiment, one, 2, 3, 4, 5, 6, 7, 8, 9, 10, 11 or 12 of the variable region amino acid residues 90-101 of SEQ ID NO: 2 are independently substituted with at least one or more different amino acids, and wherein the length of CDRL3 of the antibody is greater than or equal to 12 amino acid residues. In another embodiment, the variable region amino acid residues 90-101 of SEQ ID NO: 2 are independently substituted with at least one or more different amino acids, and wherein the length of CDRL3 of the antibody is greater than or equal to 12 amino acid residues.

In one embodiment, the antibody, or antigen binding portion thereof, has one or more of the following substitutions: (a) at least one, 2, 3, 4, 5, 6, 7, 8, 9, 10, 11 or 12 of the variable region amino acid residues 90-101 of SEQ ID NO: 2 are independently substituted with at least one or more different amino acids, and wherein the length of CDRL3 of the antibody is greater than or equal to 12 amino acid residues; (b) variable region amino acid residue 91 of SEQ ID NO: 2 is substituted with any amino acid residue except Gln; (c) variable region amino acid residue 95 of SEQ ID NO: 2 is substituted with a different aromatic amino acid residue; or (d) variable region amino acid residue 97 of SEQ ID NO: 2 is substituted with an amino acid residue selected from the group consisting of Phe, Tyr, Trp, His, Asp, Glu, Asn and Gln. In another embodiment, the variable region amino acid residues 90-101 of SEQ ID NO: 2 are independently substituted with at least one or more different amino acids, and wherein the length of CDRL3 of the antibody is greater than or equal to 12 amino acid residues.

In another embodiment, at least one or two of the variable region amino acid residues 52 and 53 of SEQ ID NO: 1 are independently substituted with an amino acid residue selected from the group consisting of Tyr, Ser, Thr, Asn, Gln, Lys and Arg. In another embodiment, the variable region amino acid residues 52 and 53 of SEQ ID NO: 1 are independently substituted with an amino acid residue selected from the group consisting of Tyr, Ser, Thr, Asn, Gln, Lys and Arg.

In another embodiment, the invention provides methods for altering the activity of an antibody that binds to the p40 subunit of IL-12 and/or IL-23, or antigen binding portion thereof, wherein said antibody or antigen binding portion thereof comprises the heavy chain variable region amino acid sequence of SEQ ID NO: 1 and the light chain variable region amino acid sequence of SEQ ID NO: 2, comprising independently substituting at least one, 2, 3, 4 or 5 of the variable region amino acid residues 33, 50, 57 and 99 of SEQ ID NO: 1 and 33 of SEQ ID NO: 2 with a different amino acid residue, thereby altering the activity of an antibody that binds to the p40 subunit of IL-12 and/or IL-23, or antigen binding portion thereof. In one embodiment, the variable region amino acid residues 33, 50, 57 and 99 of SEQ ID NO: 1 and 33 of SEQ ID NO: 2 are substituted with a different amino acid residue, thereby altering the activity of an antibody that binds to the p40 subunit of IL-12 and/or IL-23, or antigen binding portion thereof.

In one embodiment, variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys. In another embodiment, variable region amino acid residue 50 of SEQ ID NO: 1 is substituted with an amino acid residue selected from the group consisting of Phe, Tyr, Trp, His, Met, Gln, Arg and Lys. In another embodiment, variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln. In another embodiment, variable region amino acid residue 99 of SEQ ID NO: 1 is substituted with an amino acid residue selected from the group consisting of Phe, Tyr, Trp, His, Met, Arg and Lys. In another embodiment, variable region amino acid residue 33 of SEQ ID NO: 2 is substituted with an amino acid residue selected from the group consisting of Phe, Tyr, Trp, His, Gln and Lys.

In one embodiment, variable region amino acid residue 33 of SEQ ID NO: 1 is substituted with Lys. In another embodiment, variable region amino acid residue 50 of SEQ ID NO: 1 is substituted with Tyr or Trp. In another embodiment, variable region amino acid residue 57 of SEQ ID NO: 1 is substituted with Ile or Trp. In another embodiment, variable region amino acid residue 57 of SEQ ID NO: 1 is substituted with Ser or Thr. In another embodiment, variable region amino acid residue 99 of SEQ ID NO: 1 is substituted with Tyr or Trp. In another embodiment, variable region amino acid residue 33 of SEQ ID NO: 2 is substituted with Tyr or Trp.

In one embodiment, the invention provides an isolated antibody, or antigen binding portion thereof, produced according to the methods described herein.

In another embodiment, the invention provides an isolated antibody, or antigen-binding portion thereof, that binds to the p40 subunit of IL-12 and/or IL-23, or antigen binding portion thereof, wherein said antibody binds to a conformational epitope comprising at least one amino acid residue or at least two or three amino acid residues selected from the group consisting of amino acid residues 16, 87 and 93 of the amino acid sequence of SEQ ID NO: 3, or within 1-10 Å of said amino acid residue. In one embodiment, the antibody or antigen-binding portion thereof binds to amino acid residue 16. In one embodiment, the antibody or antigen-binding portion thereof binds to amino acid residues 16, 87 and 93 of SEQ ID NO: 3.

In another embodiment, the isolated antibody, or antigen binding portion thereof, binds to the p40 subunit of IL-12 and/or IL-23 with a K_{off} of 1×10⁻³ M⁻¹ or less or a K_{d} of 1×10⁻¹⁰ M or less.

In another embodiment, the isolated antibody, or antigen binding portion thereof, neutralizes the biological activity of the p40 subunit of IL-12 and/or IL-23.

In one embodiment, the antibody, or antigen-binding portion thereof, of the invention does not include any antibody known in the art prior to the present invention to bind to the epitopes discussed herein. For example, in one embodiment, the antibody, or antigen-binding portion thereof, is not an antibody described in U.S. Patent Publication No. 2009/0202549, the entire contents of which are hereby expressly incorporated herein In another embodiment, the antibody, or antigen-binding portion thereof, is not an antibody described in U.S. Pat. No. 6,902,734 or U.S. Pat. No. 7,166,285, the entire contents of each of which are hereby expressly incorporated herein. In another embodiment, the antibody, or antigen-binding portion thereof, is not the antibody C340 described in U.S. Pat. No. 6,902,764 or U.S. Pat. No. 7,166,285, the entire contents of which are hereby expressly incorporated herein.

In another aspect, the invention provides a method for inhibiting the activity of IL-12 and/or IL-23 in a subject suffering from a disorder in which the activity of IL-12 and/or IL-23 is detrimental, comprising administering to the subject an antibody, or antigen binding portion thereof, of the invention, such that the activity of IL-12 and/or IL-23 in the subject is inhibited. In one embodiment, an effective amount of the antibody is administered to the subject.

In a related aspect, the invention provides a method for treating a subject suffering from a disorder in which the activity of IL-12 and/or IL-23 is detrimental, comprising administering to the subject an antibody, or antigen binding portion thereof, of the invention, thereby treating the subject. In one embodiment, an effective amount of the antibody is administered to the subject.

In another aspect, the invention provides a use of an antibody, or antigen binding portion thereof, of the invention in therapy. In another aspect, the invention provides a use of an antibody, or antigen binding portion thereof, of the invention for treating a disorder in which the activity of IL-12 and/or IL-23 is detrimental. In another aspect, the invention provides a use of an antibody, or antigen binding portion thereof, of the invention in the manufacture of a medicament for the treatment of a disorder in which the activity of IL-12 and/or IL-23 is detrimental. In another aspect, the invention provides a use of an antibody, or antigen binding portion thereof, of the invention for inhibiting the activity of IL-12 and/or IL-23 in a subject suffering from disorder in which the activity of IL-12 and/or IL-23 is detrimental. In another aspect, the invention provides a use of an antibody, or antigen binding portion thereof, of the invention in the manufacture of a medicament for inhibiting the activity of IL-12 and/or IL-23 in a subject suffering from disorder in which the activity of IL-12 and/or IL-23 is detrimental.

In one embodiment, the disorder in which the activity of IL-12 and/or IL-23 is detrimental is a disorder selected from the group consisting of psoriasis, rheumatoid arthritis, Crohn's disease, Multiple Sclerosis and psoriastic arthritis. In one embodiment, the disorder in which the activity of IL-12 and/or IL-23 is detrimental is psoriasis. In one embodiment, the disorder in which the activity of IL-12 and/or IL-23 is detrimental is rheumatoid arthritis. In one embodiment, the disorder in which the activity of IL-12 and/or IL-23 is detrimental is Crohn's disease. In one embodiment, the disorder in which the activity of IL-12 and/or IL-23 is detrimental is Multiple Sclerosis. In one embodiment, the disorder in which the activity of IL-12 and/or IL-23 is detrimental is psoriatic arthritis.

In one embodiment, the disorder in which the activity of IL-12 and/or IL-23 is detrimental is a disorder selected from the group consisting of sarcoidosis, palmo-plantar pustular psoriasis, and palmo-plantar pustulosis, severe palmar plantar psoriasis, active ankylosing spondylitis and primary biliary cirrhosis. In one embodiment, the disorder in which the activity of IL-12 and/or IL-23 is detrimental is sarcoidosis. In one embodiment, the disorder in which the activity of IL-12 and/or IL-23 is detrimental is palmo-plantar pustular psoriasis. In one embodiment, the disorder in which the activity of IL-12 and/or IL-23 is detrimental is palmo-plantar pustulosis. In one embodiment, the disorder in which the activity of IL-12 and/or IL-23 is detrimental is severe palmar plantar psoriasis. In one embodiment, the disorder in which the activity of IL-12 and/or IL-23 is detrimental is spondylitis. In one embodiment, the disorder in which the activity of IL-12 and/or IL-23 is detrimental is primary biliary cirrhosis.

In one embodiment, the disorder in which the activity of IL-12 and/or IL-23 is detrimental is an autoimmune disease. In one embodiment, the autoimmune disease is associated with inflammation, including, without limitation, rheumatoid spondylitis, allergy, autoimmune diabetes, autoimmune uveitis.

In one embodiment, the disorder in which the activity of IL-12 and/or IL-23 is detrimental is a disorder selected from the group consisting of rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjodgren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 depicts the heavy and light chain variable region amino acid sequences of a human antibody that binds human IL-12p40, J695. Kabat numbering is used to identify amino acid positions.

FIG. 2 depicts the CDR sequences and functional characteristics of J695 and selected precursor antibodies.

FIG. 3 depicts the unique hairpin conformation of J695 CDR L3 enabling phosphate ion coordination at the center of the combining site. CDR L3 of J695 (Fab Crystal Form I), which contains the cis His-L95A-Pro0L95B peptide bond, and select other residues are shown, along with tightly-bound water molecules (red spheres) and the phosphate ion (orange/red). Hydrogen bonds are shown as grey lines.

FIG. 4 depicts J695 CDR L3 adopting a non-canonical conformation. Superposition of J695 CDR L3 (Fab crystal Form I) with that from a representative structure of canonical class 1 (Al-Lazikani, Lesk et al. 1997) (4-4-20 Fab, pdb entry 1flr (Whitlow, Howard et al. 1995)). CDR L3 is more extended in J695 and has a bulge centered at Pro-L95B, which both alter the position of the conserved proline residue.

FIG. 5 depicts surface representations of the J695 antigen-binding site (Fab crystal Form II), showing that J695 and IL-12 p40 possess complementary charged surfaces, in particular, showing the highly electropositive binding cleft of the J695 binding site. The solvent accessible surface is colored according to electrostatic surface potential (blue, white, red: +15, 0, −15 kT/e). The left-hand view is from the side of the antigen-binding site, and the right-hand view is from directly above. Inset: Fab crystal Form I.

FIG. 6 depicts a surface representation of IL-12 p70, showing its highly electronegative surface patches. The electrostatic scale and coloring is: blue, white, red: +15, 0, −15 kT/e, respectively; the p35 subunit is tinted light-green. The N-terminus of IL-12 p40 is at left, and the C-terminus is at the right. Antibody binding sites discussed in the specification are highlighted.

FIG. 7 depicts J695 binding to the p40 subunit of IL-12 p70. In this figure, based on the J695 Fab/IL-12 p70 complex crystal structure, the J695 Fab light chain is colored light blue and the heavy chain is colored dark blue. Each CDR is a distinct color. The IL-12 p40 subunit is tan, and the p35 subunit is light-green. The primary loops on p40 that interact with J695, mostly in domain D1, are each a distinct color.

FIG. 8 depicts J695 binding IL-12 p40 at multiple sites. In this figure, based on the J695 Fab/IL-12 p70 complex crystal structure, the J695 Fab is colored light (light chain) and dark (heavy chain) blue; each CDR is a distinct color. The IL-12 p40 subunit is tan. Various key contact residues on J695 and IL-12 p40 are labeled; IL-12 p40 Loops 1, 3, and 4 are indicated.

FIG. 9 depicts the surface representation of the J695 combining site. In this figure, based on the J695 Fab/IL-12 p70 complex crystal structure, each CDR is colored distinctly. The view is from the position of bound IL-12 p40. IL-12 p40 residue Asp87 (side chain atoms shown as spheres) inserts deeply into a pocket formed by CDRs L1, L2, L3, and H3.

FIG. 10 is a crystal structure depicting that a large gap exists between J695 and IL-12 p40 at the combining site. (Top) The J695 surface, viewed from the side (rotated ˜90° from FIG. 9). Note the deep cleft. (Bottom) Binding of p40 leaves an unfilled gap (arrow) between CDRs H2 and L3 and p40 Loops 3 and 4.

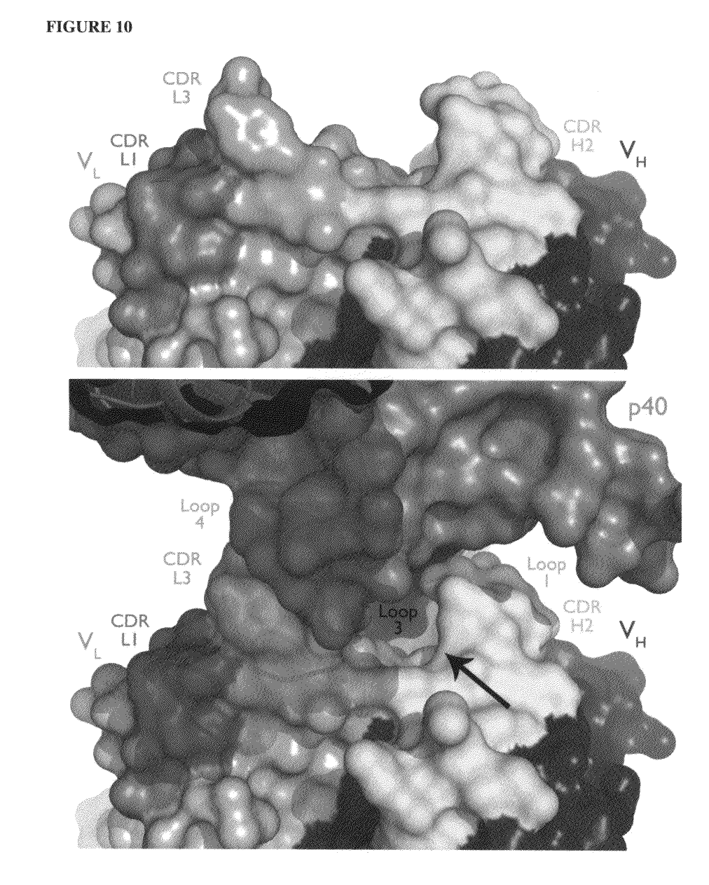

FIG. 11 depicts six antibody binding sites defined on IL-12 p40 by chimera mapping. Secondary structural elements and solvent accessibility (after (Yoon, C., S. C. Johnston, et al. 2000 “Charged residues dominate a unique interlocking topography in the heterodimeric cytokine interleukin-12.” The EMBO Journal 19 (14): 3530-3521); white, cyan and blue bar: not-, partly-, and fully-accessible) are indicated in this partial sequence alignment of p40 subunits. Identical residues are boxed in green; homologous and non-conserved residues are brown and red. Cynomolgus IL-12 p40 (not shown) is identical to rhesus p40, with the addition of a 25-residue C-terminal extension.

FIG. 12 depicts the locations of six antibody binding Sites defined on IL-12 p40 by chimera mapping. Cartoon representation based on the J695 Fab/IL-12 p70 complex crystal structure, showing the three-dimensional locations of IL-12 p40 Sites 7-12. The p40 and p35 subunits are tan and light blue; the p40 N-terminus is at right, and the C-terminus is at left. J695 Fᵥ is shown in shades of blue.

FIG. 13 is a crystal structure depicting the locations of six antibody binding sites defined on IL-12 p40 by chimera mapping. Surface representation based on the J695 Fab/IL-12 p70 complex crystal structure, showing the three-dimensional locations of IL-12 p40 sites 7-12 (FIG. 11). The p40 and p35 subunits are tan and light blue; the p40 N-terminus is at right, and the C-terminus is at left. J695 Fᵥ (cartoon) is shown in shades of blue. Inset: Back view; sites 7a, 7b, 8, 9, and 11 are visible.

FIG. 14 is a crystal structure depicting the locations of six additional Il-12 p40 Epitopes defined by chimera mapping. Surface representation based on the J695 Fab/IL-12 p70 complex crystal structure, as in FIG. 13, showing approximate locations of Epitopes 2-5. Left: Epitopes 3.1, 3.2, and 5. Right: Epitopes 2, 4a, 4b, and 4c.

FIG. 15 is a crystal structure depicting the locations of additional antibody binding sites adjacent to those defined on IL-12 p40 by chimera mapping. Surface representation based on the J695 Fab/IL-12 p70 complex crystal structure. Along with sites 7-12, as in FIG. 13, the three-dimensional locations of IL-12 p40 sites 13-18 are shown. Inset: Back view; sites 13, 14, 15, 16, and 17 are visible.

### DETAILED DESCRIPTION OF THE INVENTION

The present invention is based, at least in part, on an x-ray crystallographic study of polypeptides comprising the antigen binding fragment (Fab) of the anti-p40 subunit of IL-12/IL-23 antibody J695, alone and complexed to IL-12 p70. The atomic coordinates that result from this study are of use in identifying and designing improved antibodies and other antibody-like binding molecules (e.g., antibody fragments, domain antibodies, adnectins, nobodies, unibodies, aptamers or affibodies) that bind p40-containing cytokines such as IL-12 and IL-23. As described above, IL-23 is a heterodimeric cytokine composed of disulfide-linked p40 (the same p40 as found in IL-12) and p19 subunits.

The improved antibodies provided herein are of use in methods of treating a patient having a condition which is modulated by or dependent upon the biological activity of p40-containing cytokines, including, for example, a condition dependent on inappropriate or undesired stimulation of the immune system (multiple sclerosis, psoriasis, rheumatoid arthritis, Crohn's disease, lupus erythromatosis, chronic inflammatory diseases, and graft rejection following transplant surgery) or cancer.

In order that the present invention may be more readily understood, certain terms are first defined.

### I. Definitions

The following abbreviations and acronyms are used in this patent application. “Ab” refers to an antibody. “mAb” refers to a monoclonal antibody, “Ig” refers to an immunoglobulin. “Fab” refers to the antigen binding fragment of an antibody. “Wild-type” or “wildtype” refers to the unaltered, natural amino acid sequence of a protein.

The terms “interleukin 12” or “human interleukin 12” (abbreviated herein as IL-12 or hIL-12), as used herein, include a human cytokine that is secreted primarily by macrophages and dendritic cells. The term includes a heterodimeric protein comprising a 35 kD subunit (p35) and a 40 kD subunit (p40) which are both linked together with a disulfide bridge. The heterodimeric protein is referred to as a “p70 subunit”. The structure of human IL-12 is described further in, for example, Kobayashi, et al. (1989) J. Exp Med. 170:827-845; Seder, et al. (1993) Proc. Natl. Acad. Sci. 90:10188-10192; Ling, et al. (1995) J. Exp Med. 154:116-127; Podlaski, et al. (1992) Arch. Biochem. Biophys. 294:230-237. The term human IL-12 is intended to include recombinant human IL-12 (rh IL-12), which can be prepared by standard recombinant expression methods.

Interleukin-12 (IL-12) is an early, pro-inflammatory cytokine secreted by Ag-presenting cells that stimulates cell-mediated immunity to intracellular pathogens (Wolf, S. F., P. A. Temple, et al. (1991). “Cloning of cDNA for natural killer cell stimulatory factor, a heterodimeric cytokine with multiple biologic effects on T and natural killer cells.” J Immunol 146 (9): 3074-81; D'Andrea, A., M. Rengaraju, et al. (1992). “Production of natural killer cell stimulatory factor (interleukin 12) by peripheral blood mononuclear cells.” J. Exp. Med. 176: 1387-1398; Trinchieri, G. (1998). “Interleukin-12: a cytokine at the interface of inflammation and immunity.” Advanced Immunology 70: 83-243). The involvement of cytokines in a variety of autoimmune diseases such as rheumatoid arthritis, Crohn's disease, and multiple sclerosis has been well-established (Flavell, R. A. (2002). “The relationship of inflammation and initiation of autoimmune disease: role of TNF super family members.” Curr Top Microbiol Immunol 266: 1-9; O'Shea, J. J., A. Ma, et al. (2002). “Cytokines and autoimmunity.” Nat Rev Immunol 2 (1): 37-45). In particular, unregulated IL-12 secretion results in inappropriate autoimmune responses, for example in Crohn's disease (Tsukada, Y., T. Nakamura, et al. (2002). “Cytokine profile in colonic mucosa of ulcerative colitis correlates with disease activity and response granulocytapheresis.” The American Journal of Gastroenterology 97 (11): 2820-2828).

The terms “interleukin 23” or “human interleukin 23” (abbreviated herein as IL-23 or hIL-23), as used herein, include a human heterodimeric cytokine protein that consists of two subunits, p19 (the IL-23 alpha subunit), and p40 which is the beta subunit of IL-12 (i.e., IL-12B). IL-23 is secreted by a number of different cells including macrophages and dendritic cells. IL-23, like IL-12, appears to be important in the development of autoimmune diseases; for example, it plays a key role in a murine model of multiple sclerosis (Cua, D. J., J. Sherlock, et al. (2003). “Interleukin-23 rather than interleukin-12 is the critical cytokine for autoimmune inflammation of the brain.” Nature 421 (6924): 744-8). The receptor of IL23 is formed by the beta 1 subunit of IL12 (IL12RB1) and an IL23 specific subunit, IL23R. Both IL23 and IL12 can activate the transcription activator STAT4, and stimulate the production of interferon-gamma (IFNG). In contrast to IL12, which acts mainly on naive CD4(+) T cells, IL23 preferentially acts on memory CD4(+) T cells. IL-23 is an important part of the inflammatory response against infection. It promotes upregulation of the matrix metalloprotease MMP9, increases angiogenesis and reduces CD8+ T-cell infiltration. Recently, IL-23 has been implicated in the development of cancerous tumors. In conjunction with IL-6 and TGF-β1, IL-23 stimulates naive CD4+ T cells to differentiate into a novel subset of cells called Th17 cells, which are distinct from the classical Th1 and Th2 cells. Knockout mice deficient in either p40 or p19, or in either subunit of the IL-23 receptor (IL-23R and IL12R-β1) develop less severe symptoms of multiple sclerosis and inflammatory bowel disease highlighting the importance of IL-23 in the inflammatory pathway.

An “epitope” is a term of art that indicates the site or sites of interaction between an antibody and its antigen(s). As described by (Janeway, C., Jr., P. Travers, et al. (2001). Immunobiology: the immune system in health and disease. Part II, Section 3-8. New York, Garland Publishing, Inc): “An antibody generally recognizes only a small region on the surface of a large molecule such as a protein . . . [Certain epitopes] are likely to be composed of amino acids from different parts of the [antigen] polypeptide chain that have been brought together by protein folding. Antigenic determinants of this kind are known as conformational or discontinuous epitopes because the structure recognized is composed of segments of the protein that are discontinuous in the amino acid sequence of the antigen but are brought together in the three-dimensional structure. In contrast, an epitope composed of a single segment of polypeptide chain is termed a continuous or linear epitope” (Janeway, C., Jr., P. Travers, et al. (2001). Immunobiology: the immune system in health and disease. Part II, Section 3-8. New York, Garland Publishing, Inc).

As used herein, the terms “conformational epitope” or “non-linear epitope” or “discontinuous epitope” are used interchangeably to refer to an epitope which is composed of at least two amino acids which are not consecutive amino acids in a single protein chain. For example, a conformational epitope may be comprised of two or more amino acids which are separated by a stretch of intervening amino acids but which are close enough to be recognized by an antibody of the invention as a single epitope. As a further example, amino acids which are separated by intervening amino acids on a single protein chain, or amino acids which exist on separate protein chains, may be brought into proximity due to the conformational shape of a protein structure or complex to become a conformational epitope which may be bound by an antibody of the invention. Particular discontinuous and conformation epitopes are described herein.

It will be appreciated by one of skill in the art that, in general, a linear epitope bound by an antibody of the invention may or may not be dependent on the secondary, tertiary, or quaternary structure of the antigen, e.g., IL-12 or IL-23. For example, in some embodiments, an antibody of the invention may bind to a group of amino acids regardless of whether they are folded in a natural three dimensional protein structure. In other embodiments, an antibody of the invention may not recognize the individual amino acid residues making up the epitope, and may require a particular conformation (bend, twist, turn or fold) in order to recognize and bind the epitope.

As used herein, the term “loop” is used to refer to a turn in the secondary structure of a protein, wherein two Cα atoms closely approach each other (e.g., within about 7 Å or less) and are not involved in a regular secondary structure element such as an alpha helix or beta sheet. A loop may be extended and/or disorded without well-formed or fixed internal hydrogen bonding. A loop may include a turn in which two Cα atoms are separated by two, three, four, five or more residues.

The term “atomic coordinates” (or “structural coordinates” or “atomic model”) is a term of art that refers to mathematical three-dimensional coordinates of the atoms in the material derived from mathematical equations related to the patterns obtained on diffraction of x-rays by the atoms (x-ray scattering centers) of a crystalline material. The diffraction data are used to calculate an electron density map of the unit cell of the crystal. These electron density maps are used to establish the positions of the individual atoms within the unit cell of the crystal. Atomic coordinates can be transformed, as is known to those skilled in the art, to different coordinate systems without affecting the relative positions of the atoms. Such transformed atomic coordinates should be considered as equivalent to the original coordinates.

Unless otherwise indicated, the terms “antibody” and/or “antibodies” are used collectively to refer to an antibody, including whole antibodies and any antigen binding fragment (i.e., “antigen-binding portion”) or single chains thereof, and antibody variants, including bispecific, heterospecific, and heteroconjugate forms. Antibodies of the invention may be polyclonal, monoclonal, chimeric, humanized or human. Also included are any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof. The term “antibody” is also used herein to refer to antibody-like binding molecules or “antibody mimetics”, e.g., molecules that mimic the structure and/or function of an antibody, or fragment or portion thereof, but which are not limited to native antibody structures. Such antibody-like molecules include, for example, domain antibodies, adnectins, nanobodies, versabodies, unibodies, affibodies, avimers, anticalins, DARPins, peptidic molecules and aptamers.

In one embodiment, an “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., the p40 subunit of IL-12 and/or IL-23). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3rd ed. 1993); (iv) a Fd fragment consisting of the V_{H} and C_{H}1 domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The amino acids that make up antibodies described or encompassed herein are often abbreviated. The amino acid designations can be indicated by designating the amino acid by its single letter code, its three letter code, or name as is well understood in the art (Alberts, B., A. Johnson, et al. (2002). Molecular Biology of The Cell. New York, Garland Publishing, Inc.):

| Single Letter | Three Letter | |
|---|---|---|
| Code | Code | Name |
| A | Ala | Alanine |
| C | Cys | Cysteine |
| D | Asp | Aspartic acid |
| E | Glu | Glutamic acid |
| F | Phe | Phenylanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Furthermore, amino acid sequences described herein include “conservative mutations,” including the substitution, deletion or addition of nucleic acids that alter, add or delete a single amino acid or a small number of amino acids in a coding sequence where the nucleic acid alterations result in the substitution of a chemically similar amino acid. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N) and glutamine (Q); N, Q, serine (S), threonine (T), and tyrosine (Y); K, R, H, D, and E; D, E, N, and Q; alanine (A), valine (V), leucine (L), isoleucine (I), proline (P), phenylalanine (F), tryptophan (W), methionine (M), cysteine (C), and glycine (G); F, W, and Y; H, F, W, and Y; C, S and T; C and A; S and T; S, T, and Y; V, I, and L; V, I, and T. Other conservative amino acid substitutions are also recognized as valid, depending on the context of the amino acid in question. For example, in some cases, methionine (M) can substitute for lysine (K). In addition, sequences that differ by conservative variations are generally homologous.

An “isolated antibody”, as used herein, is intended to refer to an antibody that is substantially free of other antibodies having different antigenic specificities (e.g., an isolated antibody that specifically binds to a p40 subunit of IL-12/IL-23 is substantially free of antibodies that specifically bind antigens other than the p40 subunit of IL-12/23). Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences. It will be appreciated by one of skill in the art that when a sequence is “derived” from a particular species, said sequence may be a protein sequence, such as when variable region amino acids are taken from a murine antibody, or said sequence may be a DNA sequence, such as when variable region encoding nucleic acids are taken from murine DNA. A humanized antibody may also be designed based on the known sequences of human and non-human (e.g., murine or rabbit) antibodies. The designed antibodies, potentially incorporating both human and non-human residues, may be chemically synthesized. The sequences may also be synthesized at the DNA level and expressed in vitro or in vivo to generate the humanized antibodies.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” or “antibody mimic” is intended to refer to molecules capable of mimicking an antibody's ability to bind an antigen, but which are not limited to native antibody structures. Examples of such antibody mimetics include, but are not limited to, Domain antibodies, Adnectins (i.e., fibronectin based binding molecules), Affibodies, DARPins, Anticalins, Avimers, Nanobodies, Unibodies, Versabodies, Aptamers and Peptidic Molecules, all of which employ binding structures that, while they mimic traditional antibody binding, are generated from and function via distinct mechanisms. The embodiments of the instant invention, as they are directed to antibodies, or antigen binding portions thereof, also apply to the antibody mimetics described above.

Amino acid substitution (“point”) mutations are represented by the wild-type amino acid residue type, the residue number, and the mutated amino acid residue type. For example, point mutation of glycine 96 to asparagine is represented as either “Gly-96-Asn” or “G96N”, using the standard three- or one-letter abbreviations for amino acids.

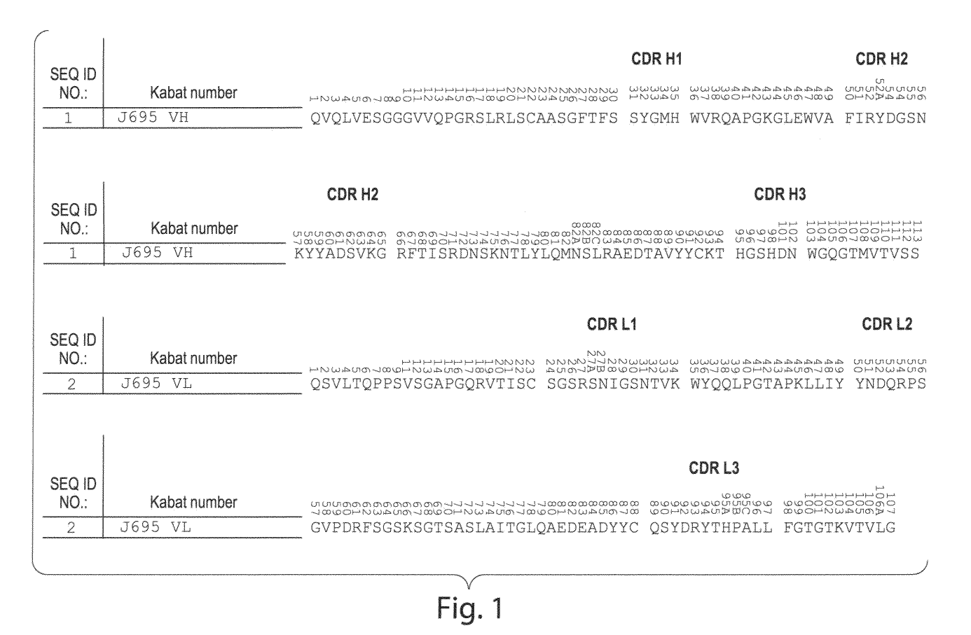

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For example, for the human anti-p40 subunit of IL-12/IL-23 antibody J695 referenced herein, the hypervariable regions are as follows. For the heavy chain variable region, the hypervariable region ranges from amino acid positions 27 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3. (See Kabat numbering for J695 shown in FIG. 1).

The term “activity” includes activities such as the binding specificity/affinity of an antibody for an antigen, for example, an anti-hIL-12 antibody that binds to an IL-12 antigen and/or the neutralizing potency of an antibody, for example, an anti-hIL-12 antibody whose binding to hIL-12 inhibits the biological activity of hIL-12, e.g. inhibition of PHA blast proliferation or inhibition of receptor binding in a human IL-12 receptor binding assay

The term “modifying”, as used herein, is intended to refer to changing one or more amino acids in the antibodies or antigen-binding portions thereof. The change can be produced by adding, substituting or deleting an amino acid at one or more positions. The change can be produced using known techniques, such as PCR mutagenesis.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Various aspects of the invention are described in further detail in the following subsections.

### II. Crystal Structures of J695 Fab

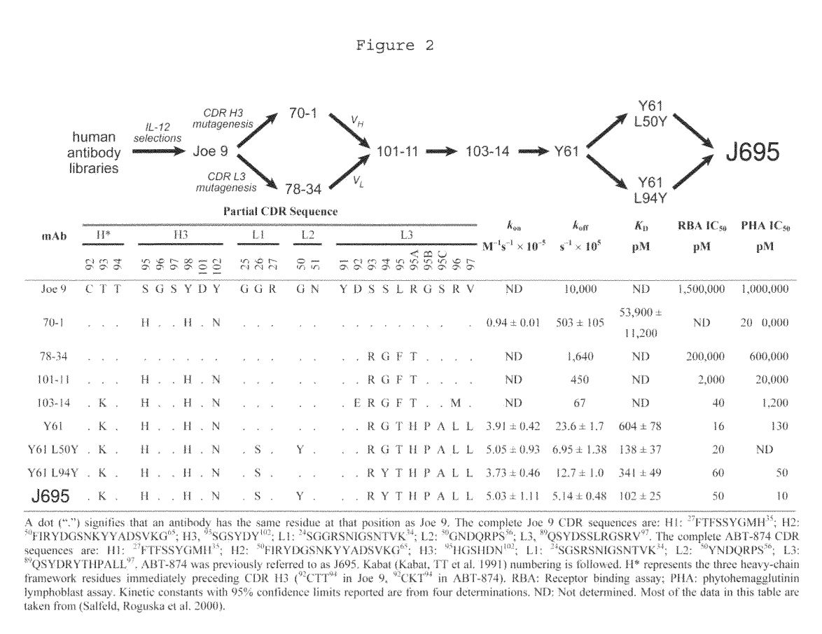

The examples herein describe the preparation and crystallization of polypeptides comprising the Fab of the human mAb J695. J695 is a recombinant human mAb against the p40 subunit of human IL-12 and human IL-23 that has therapeutic and diagnostic utility. J695 comprises IgG1 heavy and λ light chain constant region isotypes. It binds human IL-12 tightly (K_{d} 102±25 pM) and prevents its interaction with the IL-12 receptor (Salfeld et al. 1992 Science 255 (5047):959-965). Similarly, J695 binds tightly to both hp40 alone and hIL-23. The complete J695 CDR sequences are, with reference to the Kabat numbering system (See FIGS. 1 and 2): H1: ²⁷FTFSSYGMH³⁵ (aa 27-35 of SEQ ID NO:1); H2: ⁵⁰FIRYDGSNKYYADSVKG⁶⁵ (aa 50-66 of SEQ ID NO:1); H3: ⁹⁵HGSHDN¹⁰² (aa 99-104 of SEQ ID NO:1); L1: ²⁴SGSRSNIGSNTVK³⁴ (aa 23-35 of SEQ ID NO:2); L2: ⁵⁰YNDQRPS⁵⁶ (aa 51-57 of SEQ ID NO:2); L3: ⁸⁹QSYDRYTHPALL⁹⁷ (aa 90-101 of SEQ ID NO:2).

The J695 Fab fragment was prepared from CHO-cell produced J695 immunoglobulin by papain digestion followed by purification. For J695, the Fab is composed of heavy chain amino acid residues (as shown in SEQ ID NO:1) from about residue 1 to about residue 220 of SEQ ID NO:1, associated with light chain amino acid residues (as shown in SEQ ID NO:2) from about residue 1 to about residue 217 of SEQ ID NO:2. The Fab heavy and light chains are often covalently linked by a disulfide bond. Specific J695 Fab amino acid residues that make interactions with bound IL-12 p70 (p40 chain) are discussed in more detail below.

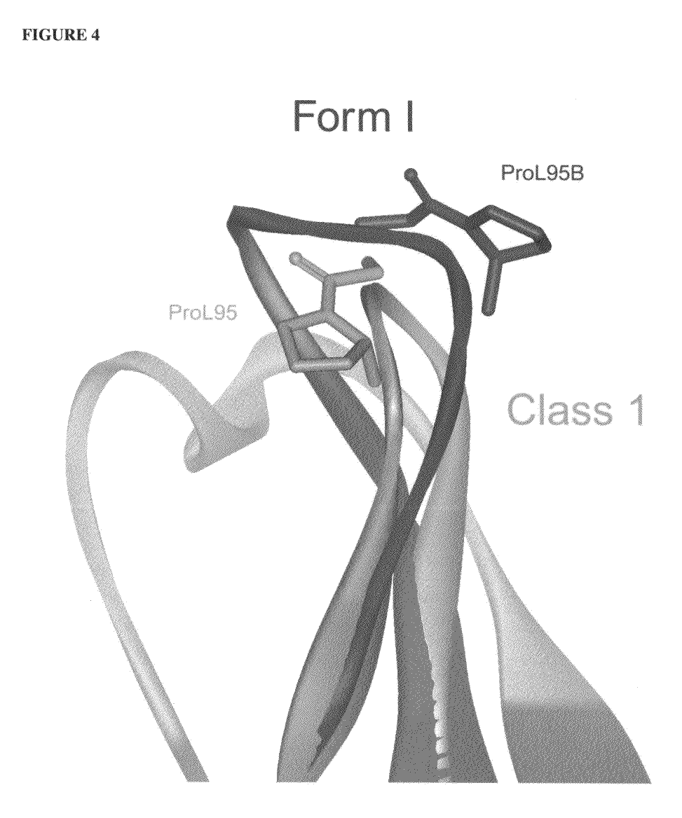

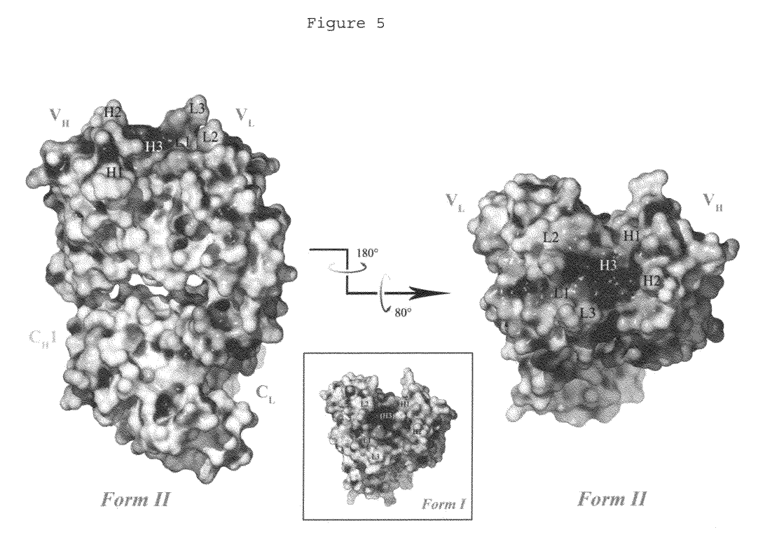

The J695 Fab was crystallized under a variety of conditions. In particular, the Fab has been crystallized in the orthorhombic space group P2₁2₁2₁, a=53.92 Å, b=67.36 Å, c=115.79 Å. This crystal form is referred to herein as “Form I” (see FIG. 4). Also in particular, the J695 Fab has been crystallized in the monoclinic space group P2₁, a=85.62 Å, b=173.41 Å, c=139.85 Å, β=105.5°. This crystal form is referred to herein as “Form II” (see FIG. 5). The term “space group” is a term of art that refers to the collection of symmetry elements of the unit cell of a crystal. The term “unit cell” is a term of art that refers to the fundamental repeating unit, akin to a building block, of a crystal. Neither of these crystalline forms have been reported previously.

Seven parameters uniquely describe the symmetry and geometrical characteristics of a crystal. These parameters are the space group (symmetry), the three unit cell axial lengths “a”, “b”, and “c”, and the three unit cell interaxial angles “α”, β”, and “γ” (geometry). “Unit cell axial length” and “unit cell interaxial angle” are terms of art that refer to the three-dimensional geometrical characteristics of the unit cell, in essence its length, width, and height, and whether the building block is a perpendicular or oblique parallelepiped. The unit cell axial lengths and interaxial angles can vary by as much as ±10% without substantively altering the arrangement of the molecules within the unit cell. Thus, when each of the unit cell axial lengths and interaxial angles is referred to herein as being “about” a particular value, it is to be understood that it is meant that any combination of these unit cell axial lengths and interaxial angles can vary by as much as ±10% from the stated values. Similarly, in particular cases, the space group of a crystal (and often in conjunction the unit cell parameters) can be altered to provide what appears to be, at first, a different crystal with altered symmetry (and geometrical) characteristics. Actually, however, this apparently new crystal is just another way of describing substantively the same crystalline form. As described below and in the Examples in detail, the J695 Fab has been crystallized in the monoclinic space group P2₁. With regard to all of the above discussion of crystal parameter variation either providing or not providing substantively the same crystals, the J695 Fab crystalline form presented herein is unique, irrespective of alternative, equally valid ways to describe substantively the same crystalline molecular arrangement.

The P2₁2₁2₁ orthorhombic unit cell reported here contains one J695 Fab molecule in the crystallographic asymmetric unit. The term “asymmetric unit” is a term of art that refers to the unique portion of a crystal's molecular contents that can be expanded, using mathematical symmetry operations that are particular to a specific space group and which are familiar to one skilled in the art, to produce first the intact unit cell, and then by application of mathematical translational symmetry operations, the entire macroscopic crystal. The P2₁ monoclinic unit cell reported here contains eight J695 Fab molecules in the crystallographic asymmetric unit. The eight unique Fabs in the Form II crystal are related to one another by non-crystallographic pseudosymmetry. In particular, two Fabs, aligned in an antiparallel fashion roughly along the <011> crystallographic direction, are related to one another by a pseudo-two-fold rotation axis (“dyad”) parallel to [100]. A second Fab pair is arrayed about the same dyad, but displaced by ˜½a. This tetrameric Fab assembly is duplicated by the translational vector [˜½a, ˜½b, ˜½c] to give the other four Fabs in the crystallographic asymmetric unit. Both of the new J695 Fab crystal forms reported herein have the advantage of providing the detailed atomic arrangement of the antigen-combining site of this antibody.

As shown by crystallographic structure determination, the J695 Fab crystals in space group P2₁2₁2₁ indeed contain not only one J695 Fab molecule in the crystallographic asymmetric unit, but also many ordered water molecules. Also as shown by crystallographic structure determination, the new J695 Fab crystals in space group P2₁ indeed contain not only eight J695 Fab molecules in the crystallographic asymmetric unit, but also many ordered water molecules.

Furthermore, as is apparent to one skilled in the art, additional crystal forms that do not differ substantively from the two crystalline forms described above can be obtained by slight modification of the protein or the crystallization conditions (such as the exact form of the protein used). These other crystals forms, which might be in different space groups, and thus appear at first glance to be distinct, should be considered as equivalent to the crystal forms reported here.

As described in the Examples, certain of these crystals were examined by x-ray crystallography and atomic coordinates for the polypeptides were obtained. The crystal structures of the J695 Fab were determined using molecular replacement and have been refined to free R-factors of 19.7% and 26.1% at 1.34-Å and 2.10-Å resolution for the Form I and Form II crystals, respectively. “Free R factor” (or “R_{free}”) is a term of art that indicates the unbiased degree of agreement between the experimentally-determined x-ray diffraction data from a crystal with theoretical diffraction data calculated from an atomic model (or atomic coordinates) constructed to explain the experimental data. R_{free} values are always greater than 0% (which indicates perfect agreement); values in the range of 10 to 30% indicate substantially correct agreement between the atomic model and the experimental data. R_{free} values typically are dependent upon the resolution of the experimentally-determined x-ray diffraction data. Lower resolution data (e.g., from 4- to 2-Å resolution) are generally associated with higher R_{free} values, whereas higher resolution data (e.g., from 1- to 2-Å resolution) are generally associated with lower R_{free} values.

1. CDR L3 of J695 Exhibits an Unusual Cis-to-Trans Peptide Bond Isomerization.

In J695 crystal Form I, CDR L3 (residues L89-L97) contains a cis-peptide bond between His-L95A^{L3} and Pro-L95B^{L3} (FIG. 2). Such a cis-proline is a conserved structural feature of CDR L3 canonical classes 1 and 2. See Chothia, C. and A. M. Lesk (1987). “Canonical Structures for the Hypervariable Regions of Immunoglobulins.” J. Mol. Biol. 196: 901-917; Chothia, C., A. M. Lesk, et al. (1989). “Conformations of immunoglobulin hypervariable regions.” Nature 342: 877-883. Al-Lazikani, B., A. M. Lesk, et al. (1997). “Standard conformations for the canonical structures of immunoglobulins.” J. Mol. Biol. 273: 927-948; Barre, S., A. S. Greenberg, et al. (1994). “Structural conservation of hypervariable regions in immunoglobulins evolution.” Structural Biology 1 (12): 915-920. In contrast, CDR L3 takes a distinct conformation in Form II, in which the His-L95A^{L3}-Pro-L95B^{L3} peptide bond adopts the trans configuration. The rearrangement of L3 brought about by this configurational switch is analogous to the induced-fit rearrangement of H3 first described for the anti-influenza virus hemagglutinin Fab 17/9 (Rini, J. M., U. Schulze-Gahmen, et al. (1992). “Structural evidence for induced fit as a mechanism for antibody-antigen recognition.” Science 255 (5047): 959-65) and the autoantibody BV04-01. Herron, J. N., X. M. He, et al. (1991). “An autoantibody to single-stranded DNA: comparison of the three-dimensional structures of the unliganded Fab and a deoxynucleotide-Fab complex.” Proteins 11 (3): 159-75. Because of this switch, the L3 CDRs of the two crystal forms superimpose poorly, with an r.m.s. deviation of 2.3 Å, whereas the other five CDRs superimpose well, with r.m.s. deviation's of 0.2-0.4 Å.

A systematic, algorithmic search of the Protein Data Bank (453 Ab structure entries available as of 28 Mar. 2003) (Berman, H. M., T. Battistuz, et al. (2002). “The Protein Data Bank.” Acta Cryst. D58: 899-907) was performed to identify examples of cis-to-trans peptide bond isomerization, both in the antibodies as a whole but especially within the CDRs. The algorithm used herein, which allowed the elimination of a large number of spurious cis/trans pairs, identified just one prior example of this phenomenon observed with J695, namely the anti-single stranded DNA mAb DNA-1 (Tanner, J. J., A. A. Komissarov, et al. (2001). “Crystal Structure of an Antigen-binding Fragment Bound to Single-stranded DNA.” J. Mol. Biol. 314: 807-822). Thus, it is believed that J695 is only the second Ab that unequivocally exhibits a peptide bond in any of the CDRs that adopts both the cis and the trans configurations, and it is the first Ab to exhibit a cis-to-trans isomerization in CDR L3.

2. CDR L3 Adopts Two Novel, Extended Hairpin Conformations

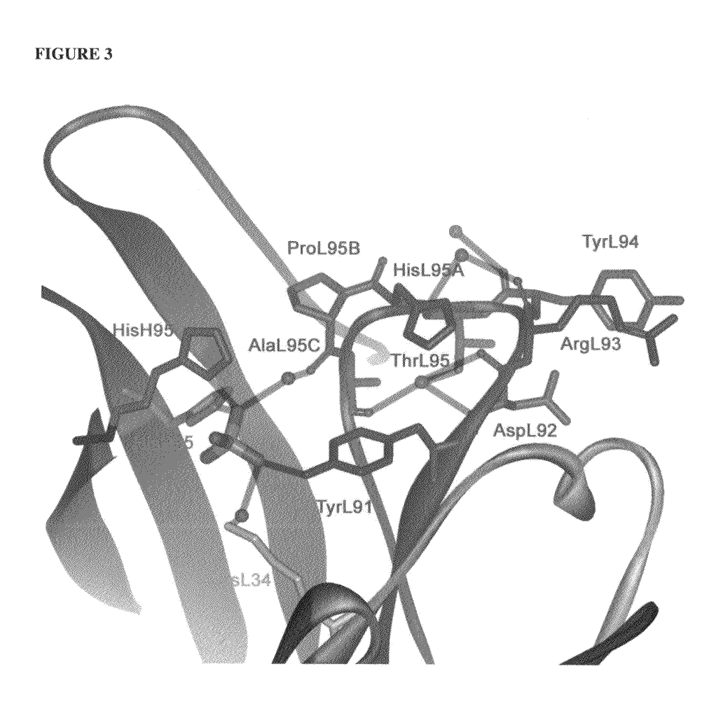

In both crystal forms, CDR L3 of J695 adopts distinct, extended hairpin conformations that have not been observed previously (FIG. 3). L3 is unusually long at 12 residues, the longest yet seen for a structurally-characterized Ab. The extraordinary length of L3 likely allows it to adopt its unusual conformations.

CDR L3 adopts a unique conformation in crystal Form I, despite the presence of the conserved cis-proline at position 95B described previously in canonical classes 1 and 2 (Chothia and Lesk 1987 Nature 342:877-883; Chothia and Lesk 1989 Nature 342:877-883; Barre and Greenberg 1994 Structural Biology 1 (12):915-920; Al-Lasikani and Lesk 1997 J. Mol. Biol. 273:927-948) because of its three-residue extension and lack of the conserved residue Gln-L90. The L3 conformation also does not correspond to any of the newer canonical clusters described by Martin and Thorton (Martin and Thornton 1996 J. Mol. Biol. 263:800-815) nor does it resemble any of the novel, non-cluster loop structures they documented. The extra residues allow L3 to extend from the framework and form a bulge around Pro-L95B^{L3}, thereby delimiting one end of the antigen-binding site. In this conformation, the cis-proline has flipped relative to the conformation observed in canonical class 1 so that the C_{β} atom is pointing toward the antigen-binding site rather than away from it (FIG. 4).

Three tightly-bound water molecules stabilize the extended L3 conformation. One water molecule in the center of the L3 hairpin, which plays a structural role similar to that of the usually-conserved Gln-L90, forms hydrogen bonds to the side-chain of Thr-L95^{L3} (3.0 Å), the main-chain carbonyl oxygen atoms of Asp-L92^{L3} (3.1 Å) and Ala-L95C^{L3} (2.7 Å), and the amide nitrogen of Asp-L92^{L3} (2.9 Å) (FIG. 3). The second water, located at the tip of the hairpin, forms hydrogen bonds to the carbonyl oxygen of Arg-L93^{L3} (3.1 Å) and the amide nitrogen of His-L95A^{L3} (2.7 Å), and the third forms a hydrogen bond (2.8 Å) to the carbonyl oxygen of Tyr-L94^{L3}. The cis-peptide bond also helps to form this novel structure. A bound phosphate (or sulfate) links the L1, L3, H2 and H3CDRs (FIG. 3) through direct and water-mediated interactions with the N^{ζ} atom of Lys-L34^{L1}, the carbonyl oxygen of Pro-L95B^{L3}, Tyr-L91^{L3} O^{η}, His-H35^{H1} N^{ε1}, and His-H95^{H3} N^{δ1}.

CDR L3 adopts a distinct, also non-canonical conformation in crystal Form II, in part due to isomerization of the His-L95A^{L3}-Pro-L95B^{L3} peptide bond to the trans configuration. The L3 conformation is rigidified by hydrogen bond interactions with several tightly-bound water molecules, in a fashion similar to Form I, but with loss of the hydrogen bond to the side chain of Thr-L95^{L3}. Water-mediated interactions distinct from those seen in Form I include bridging hydrogen bonds to the side chain of Gln-L31^{L1} and several main chain atoms of Thr-L95^{L3} and His-L95A^{L3}.

3. Insertion of CDR L3 into the Combining Site of a Second Fab Mimics Antigen Binding

Insertion of L3 from one molecule in the crystal lattice into the antigen-binding site of a second molecule reinforces the L3 conformation in crystal Form II. This intermolecular contact, which is not found in Form I, wedges L3 between L3′ and H3′ from the crystallographic symmetry-related Fab. This reciprocal L3 exchange displaces the bound phosphate anion observed in crystal Form I; the resulting void is filled by a general inward “tightening” of the CDRs, two well-ordered water molecules, and the side chain of Tyr-L′94^{L3}.

The reorganization of the tip of CDR L3 in Form II, caused by the cis-trans isomerization and the ensuing formation of extensive crystal packing contacts, can be described as a rotation of residues from Arg-L93^{L3} to His-L95A^{L3} by 153° into the antigen-binding cleft. This rotation, about an axis approximately defined by the Arg-L93^{L3} C^{α} and the pyrrolidine ring of Pro-L95B^{L3}, shifts Thr-L95^{L3} by over 9 Å toward the antigen-binding site. The C^{α} atom of Tyr-L94^{L3} moves 7.4 Å, and its side chain rotates into the combining site (O^{η} moves 15 Å) to form a hydrogen bond to O^{η} of the symmetry-related Tyr-L′91^{L3}. His-L95A^{L3} flips its orientation between the two crystal forms. Several additional contacts are observed in Form II between L3 and the symmetry-related H2 and H3CDRs. In contrast, CDR L3 in crystal Form I forms only a single intermolecular contact.

Thus, CDR L3 of J695 exhibits configurational isomerization that allows the Ab to present two rather different antigen combining sites to antigen. The intermolecular Ab/Ab interaction observed in crystal Form II may mimic the Ab/Ag interaction.

4. J695 Exhibits Structural Alterations at the Variable Domain Interface Characteristic of Antigen Binding

The interfaces between the variable domains in the two crystal forms differ substantially, with Form I resembling an unliganded Ab and Form II resembling a liganded Ab. First, the very short (six residues) CDR H3 is ordered in Form II only, adopting a “bulged torso” conformation (Morea et al. 1998 J. Mol. Biol 275:269-294). As discussed above, ordering of the four H3 residues H96-H101 is coupled with formation of crystal contacts that may substitute for interaction with IL-12. Ordering or conformational change of H3 upon antigen binding is commonly observed (Stanfield and Wilson 1994 Trends Biotechnol 2 (7):275-9).

Second, the solvent-accessible surface area buried at the V_{L}-V_{H} interface increases 38% from Form I to Form II (1,114 vs. 1,540±28 Å²). Such an increase is again characteristic of transformation from the unbound to the antigen-bound state (Stanfield et al. 1993 Structure 15:83-93). About two-thirds of this increase is due to ordering of H3. Consistent with the surface area differences, the V_{L}-V_{H} interface in Form I contains only one hydrogen-bonding interaction, the common buried, reciprocal exchange between the side chains of Gln-L38 and Gln-H39, whereas the interface in Form II has eight. These changes at the V_{L}-V_{H} interface contrast with the constancy of the C_{L}-C_{H}1 interface: the surface area buried between C_{L} and C_{H}1 is similar in the two crystal forms (Form I: 1,702 Å²; Form II: 1,757±159 Å², range 1,512-2,003 Å²). The relatively large variability (9%) in the Form II C_{L}-C_{H}1 interfaces, compared to the constancy (1.8%) exhibited by the variable domains, is likely due to the higher degree of disorder (reflected by higher temperature factors) in some of the Form II constant domains.

Third, the Fabs in crystal Form II exhibit a change, relative to Form I, in the pseudo-two-fold rotation axis that relates V_{L} to V_{H}. When the eight V_{L} domains of Form II are aligned on the Form I V_{L}, additional rotation must then be applied to the Form II V_{H} domains to bring them into alignment with V_{H} of Form I. These rotations average 2.1±0.9° (range 0.8-4.0). Such V_{L}-V_{H} rotational misalignment is characteristic of the differences between liganded and unliganded Fabs (Stanfield et al. 1993 Structure 15:83-93). These rotational differences are not linked to elbow angle changes, as six of the eight Form II Fabs have elbow angles identical to Form I (136±5 vs. 135°).

5. The J695 Antigen Binding Site has a Pronounced, Positively-Charged Cleft Poised to Bind a Negatively-Charged Peptide.

In both crystal forms, the J695 CDRs form a deep cleft between the light and heavy variable domains, a binding site more typical of antibodies directed against small molecule haptens (FIG. 5). In contrast, most protein-directed antibodies contain antigen-binding sites that possess a relatively flat surface (MacCallum, R. M., A. C. Martin, et al. (1996). “Antibody-antigen interactions: contact analysis and binding site topography.” J. Mol. Biol. 262 (5): 732-745). The cleft is open at both ends in crystal Form I whereas it is closed at both ends in Form II. The rearrangement of CDR L3 in Form II closes off one end of the cleft, and ordering of H3 completes the floor of the cleft and closes off the other end. The closed cleft is about 9 Å wide (V_{H} to V_{L}), ˜11 Å deep (floor to CDR tips), and ˜13 Å long (H3 to L3). The floor of the cleft is highly electropositive. Thus, J695 possesses the geometrical and charge characteristics needed to bind a negatively-charged peptide loop that extends away from the surface of IL-12.

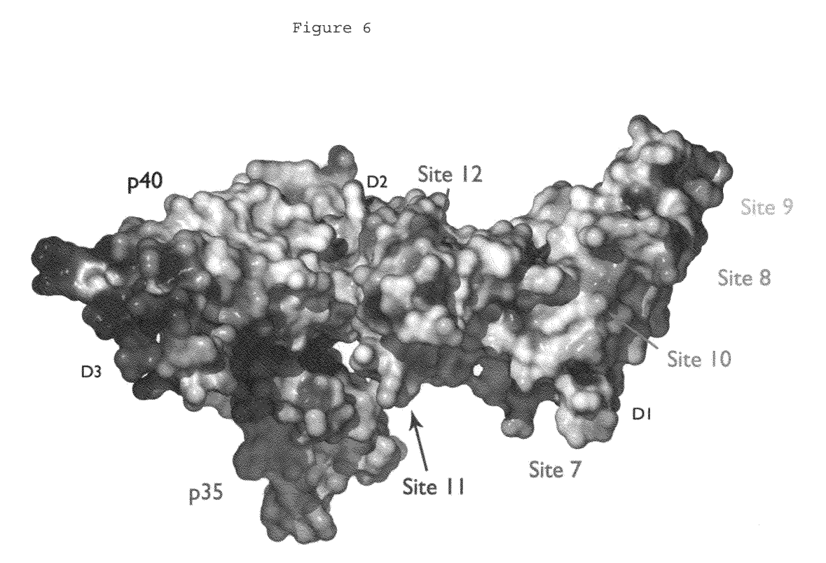

Mutations that decrease the positive charge of the J695 antigen-binding site, thereby interfering with its complementarity to negatively-charged IL-12 (FIG. 6), cause a loss in binding potency (see PCT Publication No. WO0056772 A1). Residues that contribute to the positively-charged cleft include: Asn-L31^{L1} (aa 32 of SEQ ID NO:2); Lys-L34^{L1} (aa 35 of SEQ ID NO:2); Gln-L89^{L3} (aa 90 of SEQ ID NO:2); His-H35^{H1} (aa 35 of SEQ ID NO:1); Lys-H93 (aa 97 of SEQ ID NO:1); His-H95^{H3} (aa 99 of SEQ ID NO:1); His-H98^{H3} (aa 102 of SEQ ID NO:1); Asn-H102^{H3} (aa 104 of SEQ ID NO:1); and Trp-H103 (aa 105 of SEQ ID NO:1).

CDR H3 of the J695 precursor Joe 9 lacks three of these residues. Introduction of His-H95^{H3}, and His-H98^{H3} alone brought about a five-fold improvement in binding in mAb 70-1 (FIG. 2). Combination with the repositioned L3 arginine residue in 78-34, to provide 110-11, led to a >50-fold improvement. Addition of the unusually-positioned (Morea, V., A. Tramontano, et al. (1998). “Conformations of the third hypervariable region of the VH domain of immunoglobulins.” J. Mol. Biol. 275: 269-294) framework residue Lys-H93 in 103-14 provided a 1,000-fold increase in efficacy over Joe 9. Even in the highly-optimized Y61 mutation of these positively-charged residues had a measurable impact upon IL-12 binding. For example, mutation of Y61 His-H95^{H3} to negatively-charged glutamate caused an 8-fold increase in the k_{off} rate constant (and by inference, a decrease in affinity as well), and mutation of Asn-L31^{L1} to aspartate led to a 2.5-fold increase. Thus, affinity maturation data, charge complementarity, and simple geometric considerations all indicate that J695 binds a prominent, negatively-charged loop on IL-12.

### III. Crystal Structure of J695 Fab Bound to IL-12 p70 (p40/p35)

A complex between the polypeptides comprising the Fab of the human mAb J695 and the polypeptides comprising human IL-12 p70 was prepared. As indicated above, human IL-12 p70 is composed of two subunits, a p40 polypeptide chain and a p35 polypeptide chain. The precursor (or propeptide) p40 chain amino acid residues are shown as SEQ ID NO:5. The precursor (or propeptide) p35 chain amino acid residues are shown as SEQ ID NO:6. The mature p40 chain amino acid residues, namely from about residue 23 to about residue 328 of SEQ ID NO:5, are associated with the mature p35 chain amino acid residues, namely from about residue 23 to about residue 213 of SEQ ID NO:6, to form the IL-12 p70 heterodimeric cytokine. The p40 and p35 chains are covalently linked by a disulfide bond. Henceforth, throughout this patent application the mature numbering of the IL-12 p40 and IL-12 p35 polypeptides is being used. Specific IL-12 p40 amino acid residues that make interactions with the J695 Fab are discussed in more detail below.

The amino acid sequence of native human IL-12 p40 (SEQ ID NO:5) is taken as defined in SWISS-PROT (http://www.expasy.ch; Entry Name: IL12B_HUMAN; Primary Accession Number: P29460). Amino acid residues 23 to 328 in this SWISS-PROT entry correspond to the mature IL-12 p40 polypeptide, which are referred to herein as residues 1 to 306, as shown in SEQ ID NO:3. The amino acid sequence of native human IL-12 p35 (SEQ ID NO:6) is taken as defined in SWISS-PROT (http://www.expasy.ch; Entry Name: IL12A_HUMAN; Primary Accession Number: P29459). Amino acid residues 23 to 219 in this SWISS-PROT entry correspond to the mature IL-12 p35 polypeptide, which are referred to herein as residues 1 to 197, as shown in SEQ ID NO:4.

As described in the Examples, the complex has been crystallized under a variety of conditions. In particular, the J695 Fab/IL-12 p70 complex has been crystallized in the orthorhombic space group C222₁, a=136.3151 Å, b=209.5560 Å, c=217.1127 Å. This crystalline form has not been reported previously.

As described below and in the Examples in detail, the C222₁ orthorhombic unit cell reported here contains two molecules of the J695 Fab and two molecules of IL-12 p70 in the crystallographic asymmetric unit. As shown by crystallographic structure determination, the new J695 Fab/IL-12 p70 complex crystals in space group C222₁ indeed contain not only two molecules of the J695 Fab and two molecules of IL-12 p70 in the crystallographic asymmetric unit, but also many ordered water molecules.

Furthermore, as is apparent to one skilled in the art, additional crystal forms that do not differ substantively from the orthorhombic form described above can be obtained by slight modification of the protein or the crystallization conditions (such as the exact forms of the protein used). These other crystals forms, which might be in different space groups, and thus appear at first glance to be distinct, should be considered as equivalent to the crystal forms reported here.

As described in the Examples, certain of these crystals were examined by x-ray crystallography and atomic coordinates for the polypeptides were obtained. In particular, the C222₁ crystal form report herein which was examined by x-ray crystallography has the advantage of revealing the precise molecular interactions between J695 and IL-12 p70, including the three-dimensional conformation of both molecules at the combining site, as well as which IL-12 amino acid residues comprise the binding site, or epitope. The crystal structure of the one-to-one complex between J695 Fab and IL-12 p70 was determined and refined to a free R factor of 28.7% at 3.25-Å resolution.

### IV. Antibodies that Bind the P40 Subunit of IL-12 and/or IL-23

The antibodies of the invention bind specifically to the p40 subunit of IL-12 and/or IL-23 and, preferably, to a particular domain or portion or conformational epitope of the p40 subunit described herein, such as, for example, to a portion and/or conformational epitope comprising at least one amino acid selected from residues 1-197 of the amino acid sequence of the mature human p40 protein (SEQ ID NO: 3). In a preferred embodiment, the binding of the antibodies, or antigen binding portions thereof, of the invention to the p40 subunit of IL-12 and/or IL-23 modulates, e.g., inhibits or reduces, the activity of the p40 subunit of IL-12 and/or IL-23 and/or the activity of the p40-containing cytokine. For example, the antibody, or antigen-binding portion thereof, may block the binding of the p40-containing cytokine, e.g., IL-12 or IL-23, to its receptor, e.g., the IL-12 or IL-23 receptor, respectively.

The antibodies of the invention are selected or designed to bind to specific domains or portions of the p40 subunit, for example, a portion comprising at least one amino acid selected from residues 1-197 of the amino acid sequence of the mature human p40 protein (SEQ ID NO: 3). In one embodiment, the antibodies of the invention are selected or designed to bind to a portion and/or conformational epitope of the p40 subunit comprising at least one amino acid selected from residues 1-197 of the amino acid sequence of the mature human p40 protein (SEQ ID NO: 3). In other embodiments, the antibodies of the invention are selected or designed to bind to a portion and/or conformational epitope of the p40 subunit comprising at least one amino acid residue of loops 1-7 of the p40 subunit, e.g., wherein at least one amino acid residue is selected from residues 14-23, 58-60, 84-107, 124-129, 157-164 and 194-197 of the amino acid sequence of the mature human p40 protein (SEQ ID NO: 3). In other embodiments the antibodies, or antigen binding portions thereof, are selected or designed to bind to proteins sharing homology to a domain of the p40 subunit of IL-12 and/or IL-23. For example, an antibody may be selected or designed to bind a domain which is at least 50% identical, at least 60% identical, at least 70% identical, at least 80% identical, at least 90% identical, or at least 95%, 96%, 97%, 98% or 99% identical to a domain of the p40 subunit of IL-12 and/or IL-23. Such an antibody, or antigen binding portion thereof, would be able to bind protein domains which are functionally similar to the domains of the p40 subunit of IL-12 and/or IL-23.

In one embodiment, the antibodies, or antigen-binding portions thereof, bind protein motifs which represent a contiguous string of amino acids. In other embodiments, the antibodies, or antigen binding portions thereof, bind protein motifs or consensus sequences which represent a three dimensional structure in the protein. Such motifs or consensus sequences would not represent a contiguous string of amino acids, but a non-contiguous amino acid arrangement that results from the three-dimensional folding of the p40 subunit of IL-12 and/or IL-23 (i.e., a “structural motif” or “non-linear epitope”). An example of such a motif would be Epitope 1 as described in Table 4 of section IV(C), e.g., comprising Tyr16, Asp87 and Asp93 of human p40. In one embodiment, an antibody of the present invention binds to, for example, a non-linear epitope comprising one or more amino acid residues from loops 1-7 of the p40 subunit of IL-12 and/or IL-23. Antibodies of the invention are described in further detail in the subsections below.

A. Antibodies Based on the Crystal Structure of J695 Fab/IL-12 p70 Complex

1. Contacts on IL-12 p40

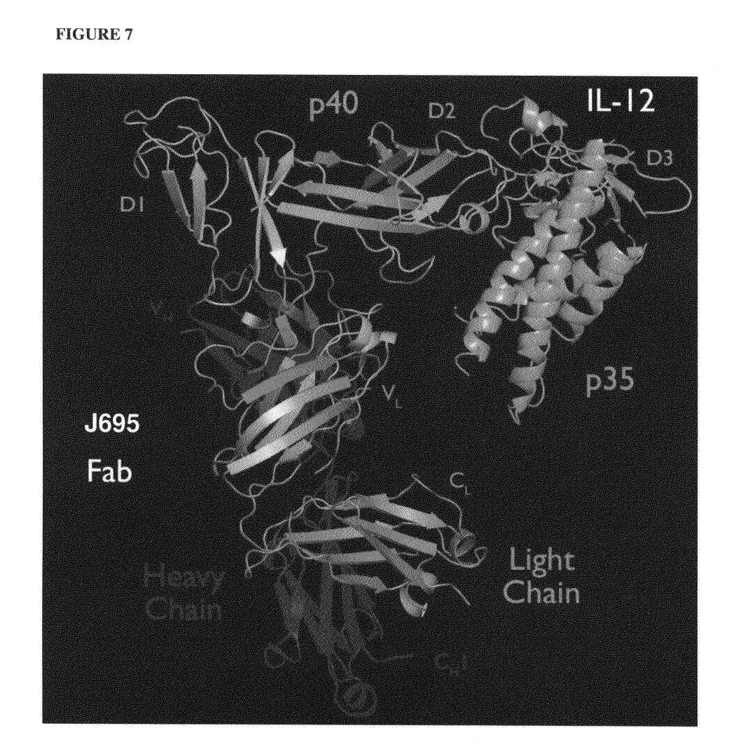

The J695 Fab/IL-12 p70 complex crystal structure indicates that J695 binds to IL-12 via the p40 subunit; there are no contacts between J695 and the p35 subunit (FIG. 7). All references to amino acid residues of the IL-12 p40 subunit are made with reference to the mature p40 polypeptide as shown in SEQ ID NO:3.

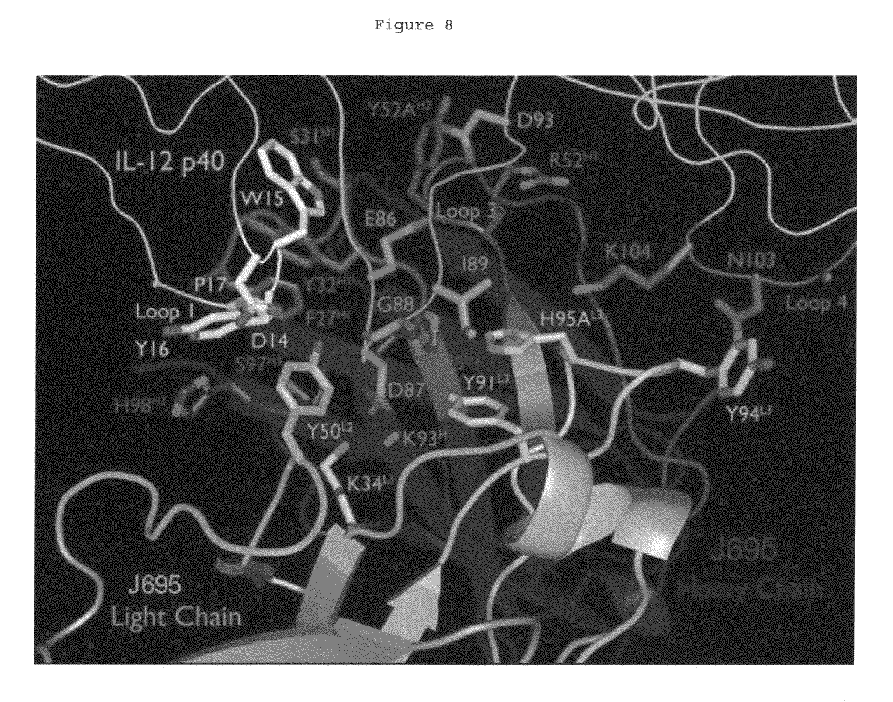

The bulk of the interactions between J695 Fab and p40 occur in the N-terminal domain D1 of p40, about amino acid residues 1 to 197, and more preferably between amino acids 1 to 107 of the mature p40 polypeptide (about residues 23 to 130 of the immature sequence; see mature p40 polypeptide sequence set forth in SEQ ID NO:3) (FIG. 8). Thus, in an exemplary embodiment, the invention provides an antibody that binds to the p40 subunit of IL-12 and/or IL-23, wherein the antibody binds to a portion and/or conformational epitope of the p40 subunit comprising at least one amino acid residue selected from amino acid residues 1-197 of SEQ ID NO:3, or within 1-10 Å of the amino acid residue. In another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue selected from amino acid residues 1-107 of SEQ ID NO:3, or within 1-10 Å of the amino acid residue.

Some interactions are also made to other domains of IL-12 p40. In particular, J695 binds to IL-12 p40 and makes contact with the following IL-12 p40 amino acid residues: Asp14, Trp15, Tyr16, Pro17, Asp18, Ala19, Pro20, Gly21, Glu22, Met23, Lys58, Glu59, Phe60, Lys84, Lys85, Glu86, Asp87, Gly88, Ile89, Trp90, Ser91, Thr92, Asp93, Ile94, Leu95, Lys96, Asp97, Gln98, Lys99, Glu100, Pro101, Lys102, Asn103, Lys104, Thr105, Phe106, Leu107, Thr124, Thr125, Ile126, Ser127, Thr128, Asp129, Arg157, Val158, Arg159, Gly160, Asp161, Asn162, Lys163, Glu164, His194, Lys195, Leu196, and Lys197 (FIG. 8). These residues are situated, respectively, in at least one loop of loops 1-7 of the p40 subunit. Therefore, also encompassed by the present invention is an antibody that binds to the p40 subunit of IL-12 and/or IL-23, wherein the antibody binds to a portion and/or conformational epitope of the p40 subunit comprising at least one amino acid residue of loops 1-7. In an exemplary embodiment, the invention provides an antibody that binds to the p40 subunit of IL-12 and/or IL-23, wherein the antibody binds to a portion and/or conformational epitope of the p40 subunit comprising at least one amino acid residue of loops 1-7, or within 1-10 Å, e.g., within 0.1, 0.5, 1, 2, 3, 4, 5, 6, 7, 8, 9 or 10 Å of the amino acid residue.

In particular, J695 binds to IL-12 p40 and makes contact with the following IL-12 p40 amino acid residues that comprise IL-12 p40 Loop 1, namely residues: Asp14, Trp15, Tyr16, Pro17, Asp18, Ala19, Pro20, Gly21, Glu22, and Met23 (FIG. 8). Accordingly, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 1 selected from the group consisting of residues 14-23, or within 1-10 Å of the amino acid residue. In an additional embodiment, the antibody binds to a portion and/or conformational epitope of the p40 subunt comprising at least one amino acid residue of loop 1 selected from the group consisting of 14-18, or within 1-10 Å of the amino acid residue. In a preferred embodiment, the antibody binds to a portion and/or conformational epitope of the p40 subunt comprising at least one amino acid residue of loop 1 selected from the group consisting of 14-17, or within 1-10 Å of the amino acid residue. In another preferred embodiment, the antibody binds to a portion and/or conformational epitope of the p40 subunt comprising at least one amino acid residue of loop 1 selected from the group consisting of 15-17, or within 1-10 Å of the amino acid residue.

The crystal structure analysis also indicates that J695 binds to IL-12 p40 and makes contact with the following IL-12 p40 amino acid residues that comprise IL-12 p40 Loop 2, namely residues: Lys58, Glu59, and Phe60. Accordingly, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 2 selected from the group consisting of residues 58-60, or within 1-10 Å of the amino acid residue.

In addition, the crystal structure analysis indicates that J695 binds to IL-12 p40 and makes contact with the following IL-12 p40 amino acid residues that comprise IL-12 p40 Loop 3, namely residues: Lys84, Lys85, Glu86, Asp87, Gly88, Ile89, Trp90, Ser91, Thr92, Asp93, and Ile94 (FIG. 8). Accordingly, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 3 selected from the group consisting of residues 84-94, or within 1-10 Å of the amino acid residue. In another embodiment, the antibody binds to a portion and/or conformational epitope of the p40 subunt comprising at least one amino acid residue of loop 3 selected from the group consisting of 85-93, or within 1-10 Å of the amino acid residue. In an additional embodiment, the antibody binds to a portion and/or conformational epitope of the p40 subunt comprising at least one amino acid residue of loop 3 selected from the group consisting of 86-89 and 93, or within 1-10 Å of the amino acid residue. In a preferred embodiment, the antibody binds to a portion and/or conformational epitope of the p40 subunt comprising at least one amino acid residue of loop 3 selected from the group consisting of 86, 87, 89 and 93, or within 1-10 Å of the amino acid residue.

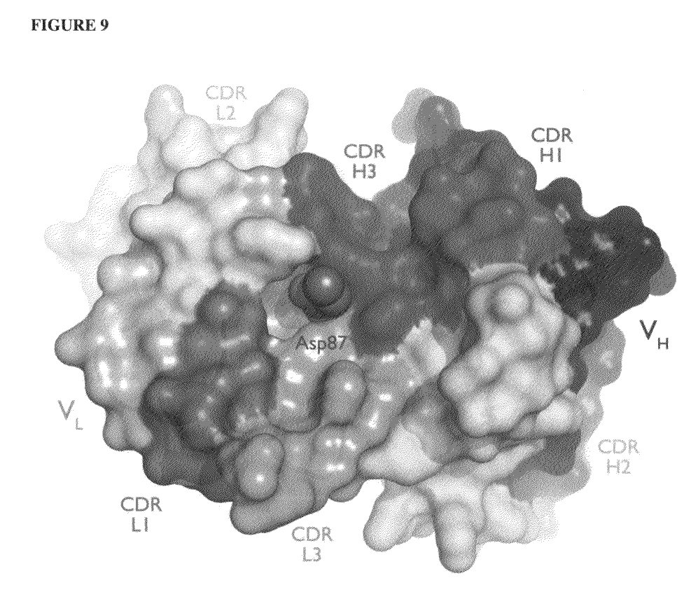

IL-12 p40 amino acid residue Asp87 is especially prominent in the binding to J695. Its side chain carboxylate binds deeply in the combining site (FIG. 9), at the same location where a bound phosphate ion was observed in the Form I crystal structure of the J695 Fab. Therefore, in an additional preferred embodiment, the antibody binds to a portion and/or conformational epitope of the p40 subunt comprising amino acid residue 87 of loop 3, or within 1-10 Å of the amino acid residue.

Furthermore, the crystal structure analysis indicates that J695 binds to IL-12 p40 and makes contact with the following IL-12 p40 amino acid residues that comprise IL-12 p40 Loop 4, namely residues: Leu95, Lys96, Asp97, Gln98, Lys99, Glu100, Pro101, Lys102, Asn103, Lys104, Thr105, Phe106, and Leu107 (FIG. 8). Accordingly, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 4 selected from the group consisting of residues 95-107, or within 1-10 Å of the amino acid residue. In another embodiment, the antibody binds to a portion and/or conformational epitope of the p40 subunt comprising at least one amino acid residue of loop 4 selected from the group consisting of 102-104, or within 1-10 Å of the amino acid residue. In a preferred embodiment, the antibody binds to a portion and/or conformational epitope of the p40 subunt comprising at least one amino acid residue of loop 4 selected from the group consisting of 103 and 104, or within 1-10 Å of the amino acid residue. In another preferred embodiment, the antibody binds to a portion and/or conformational epitope of the p40 subunit comprising amino acid residue 104 of loop 4, or within 1-10 Å of the amino acid residue. In yet another preferred embodiment, the antibody binds to a portion and/or conformational epitope of the p40 subunit comprising amino acid residue 103 of loop 4, or within 1-10 Å of the amino acid residue.

The crystal structure analysis also indicates that J695 binds to IL-12 p40 and makes contact with the following IL-12 p40 amino acid residues that comprise IL-12 p40 Loop 5, namely residues: Thr124, Thr125, Ile126, Ser127, Thr128, and Asp129 (FIG. 8). Accordingly, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 5 selected from the group consisting of residues 124-129, or within 1-10 Å of the amino acid residue.

The crystal structure analysis also indicates that J695 binds to IL-12 p40 and makes contact with the following IL-12 p40 amino acid residues that comprise IL-12 p40 Loop 6, namely residues: Arg157, Val158, Arg159, Gly160, Asp161, Asn162, Lys163, and Glu164. Accordingly, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 6 selected from the group consisting of residues 157-164, or within 1-10 Å of the amino acid residue.

The crystal structure analysis also indicates that J695 binds to IL-12 p40 and makes contact with the following IL-12 p40 amino acid residues that comprise IL-12 p40 Loop 7, namely residues: His194, Lys195, Leu196, and Lys197. Accordingly, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 7 selected from the group consisting of residues 194-197, or within 1-10 Å of the amino acid residue.

The crystal structure analysis further indicates that the majority of the specific interactions between J695 and IL-12 are the interactions with the following IL-12 p40 Loops: Loop 1, Loop 3, and Loop 4. For example, most of the specific contacts between J695 and IL-12 p70 reside in an epitope comprised primarily of four IL-12 p40 surface loops (residues 14-23, 58-60, 84-94, and 95-107; Loops 1, 2, 3, and 4, respectively, referred to above) that are not contiguous in primary sequence, a so-called “conformational” epitope (Janeway, C., Jr., P. Travers, et al. (2001). Immunobiology: the immune system in health and disease. New York, Garland Publishing, Inc). As such, in another embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loops 1-4 selected from the group consisting of residues 14-23, 58-60, 84-94, and 95-107, or within 1-10 Å of the amino acid residue. In an additional embodiment, the invention encompasses an antibody that binds to a portion and/or conformational epitope of the p40 subunit comprising at least one amino acid residue of loops 1-4 selected from the group consisting of residues 14-18, 85-93, and 102-104, or within 1-10 Å of the amino acid residue. In a further embodiment, the invention encompasses an antibody that binds to a portion and/or conformational epitope of the p40 subunit comprising at least one amino acid residue of loops 1-4 selected from the group consisting of residues 14-17, 86-89, 93, and 103-104, or within 1-10 Å of the amino acid residue. In another embodiment, the invention encompasses an antibody that binds to a portion and/or conformational epitope of the p40 subunit comprising at least one amino acid residue of loops 1-4 selected from the group consisting of residues 15-17, 86-87, 89, 93, and 104, or within 1-10 Å of the amino acid residue.

In still an additional embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loops 1-2 selected from the group consisting of residues 14-23 and 58-60, or within 1-10 Å of the amino acid residue. In another embodiment, the invention encompasses an antibody that binds to a portion and/or conformational epitope of the p40 subunit comprising at least one amino acid residue of loops 1-2 selected from the group consisting of residues 15, 17-21, 23, and 58-60, or within 1-10 Å of the amino acid residue.

In still an additional embodiment, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loop 1 selected from the group consisting of residues 14-23 and at least one amino acid residue of loop 2 selected from the group consisting of residues 58-60, or within 1-10 Å of the amino acid residue. In another embodiment, the antibody binds to a portion and/or conformational epitope of the p40 subunit comprising at least one amino acid residue of loops 1 and 3 selected from the group consisting of residues 14-23 and 84-94, or within 1-10 Å of the amino acid residue. In an additional embodiment, the antibody binds to a portion and/or conformational epitope of the p40 subunit comprising at least one amino acid residue of loop 1 selected from the group consisting of residues 14-23 and at least one amino acid residue of loop 3 selected from the group consisting of residues 84-94, or within 1-10 Å of the amino acid residue.

In further embodiments, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loops 1 and 4 selected from the group consisting of residues 14-23 and 95-107, or within 1-10 Å of the amino acid residue. In an additional embodiment, the antibody binds to a portion and/or conformational epitope of the p40 subunit comprising at least one amino acid residue of loop 1 selected from the group consisting of residues 14-23 and at least one amino acid residue of loop 4 selected from the group consisting of residues 95-107, or within 1-10 Å of the amino acid residue.

In further embodiments, the invention provides an antibody that binds to the p40 subunit of IL-12 and/or IL-23, e.g., human IL-12 and/or human IL-23, wherein the antibody binds to a portion and/or conformational epitope of the p40 subunit comprising at least one amino acid residue of loops 3 and 4 selected from the group consisting of residues 84-94 and 95-107, or within 1-10 Å of the amino acid residue. In an additional embodiment, the antibody binds to a portion and/or conformational epitope of the p40 subunit comprising at least one amino acid residue of loop 3 selected from the group consisting of residues 84-94 and at least one amino acid residue of loop 4 selected from the group consisting of residues 95-107, or within 1-10 Å of the amino acid residue.

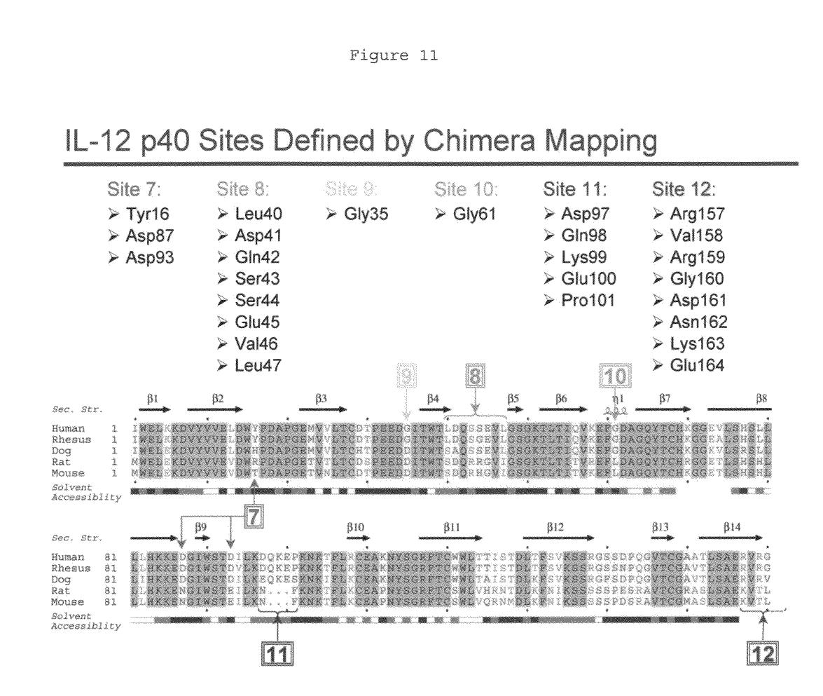

The experimentally-determined combining site between J695 and IL-12 p70 is consistent with known data concerning which p40 residues modulate binding of J695, specifically the known cross-reactivity, or lack thereof, between J695 and IL-12 p40 or IL-12 p70 from various sources, for example human, rhesus monkey, dog, rat, or mouse IL-12 (FIG. 11). For example, two key amino acid residues at the binding site are not conserved between human IL-12 and rat or mouse IL-12, namely IL-12 p40 amino acid residues Tyr16 (Loop 1) and Asp87 (Loop 3). Alteration of these two residues, namely Tyr16Arg (rat) or Tyr16Thr (mouse), and Asp87Asn (rat or mouse), as is found in rat or mouse IL-12, or in human/rat chimeric proteins (see below), essentially abrogates binding to J695.

Furthermore, deletion of IL-12 p40 amino acid residues Gln98, Lys99, and Glu100, as is found in rat or mouse IL-12 p40, alters the shapes of Loop3 and Loop4 and thus the proper presentation of the critical residues noted above to J695. The observed combining site is also consistent with the known binding of J695 to any of IL-12 p70, IL-12 p40, or IL-23 p40/p19 heterodimer, all with essentially equal affinity (FIG. 7). Finally, the observed crystallographic combining site is also consistent with known mutagenesis data from the affinity maturation of J695, i.e., which mutations made to J695 precursor antibodies affected IL-12 binding efficacy (as described in PCT Publication No. WO0056772 A1, the entire contents of which are hereby incorporated herein by reference).

In one embodiment of the invention, the antibody that binds to the p40 subunit of IL-12 and/or IL-23, or antigen-binding portion thereof, binds to a noncontinuous or conformational epitope. In one embodiment, the invention provides an antibody that binds to the p40 subunit of IL-12 and/or IL-23, wherein the antibody binds to a conformational epitope of the p40 subunit comprising at least two amino acid residues selected from amino acid residues of loops 1-7, i.e., amino acid residues 14-23, 58-60, 84-107, 124-129, 157-164 and 194-197 of the amino acid sequence of SEQ ID NO: 3, or within 1-10 Å of said amino acid residue. In one embodiment, the antibody binds to a conformational epitope of the p40 subunit comprising at least two amino acid residues selected from the amino acid residues of loop 1, i.e., amino acid residues 14-23. In one embodiment, the antibody binds to a conformational epitope of the p40 subunit comprising at least two amino acid residues selected from the amino acid residues of loop 2, i.e., amino acid residues 58-60. In one embodiment, the antibody binds to a conformational epitope of the p40 subunit comprising at least two amino acid residues selected from the amino acid residues of loop 3, i.e., amino acid residues 84-94. In one embodiment, the antibody binds to a conformational epitope of the p40 subunit comprising at least two amino acid residues selected from the amino acid residues of loop 4, amino acid residues 95-107. In one embodiment, the antibody binds to a conformational epitope of the p40 subunit comprising at least two amino acid residues selected from the amino acid residues of loop 5, i.e., amino acid residues 124-129. In one embodiment, the antibody binds to a conformational epitope of the p40 subunit comprising at least two amino acid residues selected from the amino acid residues of loop 6, i.e., amino acid residues 157-164. In one embodiment, the antibody binds to a conformational epitope of the p40 subunit comprising at least two amino acid residues selected from the amino acid residues of loop 7, i.e., amino acid residues 194-197. In another embodiment, the antibody binds to a conformational epitope of the p40 subunit comprising two or more amino acid residues selected from the amino acid residues of loops 1-7, wherein at least two of the two or more amino acid residues reside in different loops. It is to be understood that the at least two amino acid residues that reside in different loops may be from any combination of loops, e.g., loops 1 and 2, loops 1 and 3, loops 1 and 4, loops 1 and 5, loops 1 and 6, loops 1 and 7, loops 2 and 3, loops 2 and 4, loops 2 and 5, loops 2 and 6, loops 2 and 7, loops 3 and 4, loops 3 and 5, loops 3 and 6, loops 3 and 7, loops 4 and 5, loops 4 and 6, loops 4 and 7, loops 5 and 6, loops 5 and 7, or loops 6 and 7.

For example, in one embodiment, the antibody binds to a conformational epitope of the p40 subunit comprising at least one amino acid residue selected from the amino acid residues of loop 1 and at least one amino acid residue selected from the amino acid residues of loop 2. In one embodiment, the antibody binds to a conformational epitope of the p40 subunit comprising at least one amino acid residue selected from the amino acid residues of loop 1 and at least one amino acid residue selected from the amino acid residues of loop 3. In one embodiment, the antibody binds to a conformational epitope of the p40 subunit comprising at least one amino acid residue selected from the amino acid residues of loop 1 and at least one amino acid residue selected from the amino acid residues of loop 4. In one embodiment, the antibody binds to a conformational epitope of the p40 subunit comprising at least one amino acid residue selected from the amino acid residues of loop 2 and at least one amino acid residue selected from the amino acid residues of loop 3. In one embodiment, the antibody binds to a conformational epitope of the p40 subunit comprising at least one amino acid residue selected from the amino acid residues of loop 2 and at least one amino acid residue selected from the amino acid residues of loop 4. In one embodiment, the antibody binds to a conformational epitope of the p40 subunit comprising at least one amino acid residue selected from the amino acid residues of loop 3 and at least one amino acid residue selected from the amino acid residues of loop 4. It is to be understood that the conformational epitope of the p40 subunit may comprise at least two amino acid residues that reside in different loops, wherein the different loops may be any combination of loops 1, 2, 3, 4, 5, 6 and 7.

2. Contacts on J695

All of the J695 complementarity determining regions (CDRs) contact IL-12 40. In particular, binding of IL-12 occurs primarily through six regions of the overall J695 combining site, which are identified as “Sites”, as described below and in FIG. 8.

Site 1 comprises three aromatic residues (Phe, Tyr, Trp, or His), two of which are located in CDR H1 (Phe-H27 and Tyr-H32), and one of which is located in CDR H3 (His-H98), such that the Cβ atoms of these three residues form a triangle with dimensions of about 8 Å (between the two H1 residues), 11 Å and 11 Å (between each H1 residue and the H3 residue). The amino acid residues of Site 1 form a pocket into which IL-12 p40 residues Tyr16 and Pro17 are inserted, where they make numerous van der Waals interactions with J695. It is apparent from the J695/IL-12 p70 crystal structure determined here that one or more aromatic residues could be substituted for Phe-H27, Tyr-H32, or His-H98 (e.g., corresponding to amino acid residues 27, 32 and 102 of SEQ ID NO: 1, respectively) with retention or even enhancement of the binding characteristics of J695.

Site 2 comprises three residues drawn from the group of composed of Lys, Arg, Tyr, Asn, and Gln, with one residue each in CDRs L1 (Lys-L34), L3 (Tyr-L91), and H3 (including the three framework residues that proceed H3; Lys-H93), such that the Cβ atoms of these three residues form a triangle with dimensions of about 10 Å (between the L1 and L3 residues), 12 Å (between the L1 and H3 residues), and 15 Å (between the L3 and H3 residues). The amino acid residues of J695 Site 2 form a pocket into which IL-12 p40 residue Asp87 is inserted; the three J695 amino acids form specific complementary charge and hydrogen bond interactions with the Asp87 side chain carboxylate (FIG. 9). It is apparent from the J695/IL-12 p70 crystal structure determined here that one or more residues drawn from the group composed of Lys, Arg, Tyr, Asn, and Gln, could be substituted for Lys-L34 (e.g., corresponding to amino acid residue 35 of SEQ ID NO:2), Tyr-L91 (e.g., corresponding to amino acid residue 92 of SEQ ID NO:2), or Lys-H93 (e.g., corresponding to amino acid residue 97 of SEQ ID NO:1) with retention or even enhancement of the binding characteristics of J695.

Site 3 comprises two aromatic residues (Phe, Tyr, Trp, or His), both located in CDR L3 (Tyr-L91 and His-L95A), such that the Cβ atoms of these two residues are separated by about 5 Å. The amino acid residues of Site 3 form a pocket into which IL-12 p40 residue Ile89 is inserted, where it makes numerous van der Waals interactions with J695. It is apparent from the J695/IL-12 p70 crystal structure determined here that one or more aromatic residues could be substituted for Tyr-L91 or His-L95A (e.g., corresponding to amino acid residues 92 and 97 of SEQ ID NO:2, respectively) with retention or even enhancement of the binding characteristics of J695.

Site 4 comprises two residues drawn from the group of composed of Tyr, Ser, Thr, Asn, and Gln, with one residue each in CDRs L2 (Tyr-L50) and H3 (Ser-H97), such that the Cβ atoms of these two residues are separated by about 7 Å. The amino acid residues of J695 Site 4 form a pocket into which IL-12 p40 residue Asp14 is inserted; the two J695 amino acids form specific complementary charge and hydrogen bond interactions with the Asp 14 side chain carboxylate. It is apparent from the J695/IL-12 p70 crystal structure determined here that one or more residues drawn from the group composed of Tyr, Ser, Thr, Asn, and Gln, could be substituted for Tyr-L50 (e.g., corresponding to amino acid residue 51 of SEQ ID NO:2) or Ser-H97 (e.g., corresponding to amino acid residue 101 of SEQ ID NO:1) with retention or even enhancement of the binding characteristics of J695.

Site 5 comprises the entire CDR L3 of J695 (corresponding to amino acid residues 90-101 of SEQ ID NO:2), which possesses the following characteristics: (i) the length of CDR L3 is equal to or greater than 12 amino acid residues (it is 12 amino acid residues long in J695); (ii) the amino acid residue at CDR L3 position 90 is not Gln (it is Ser in J695); (iii) the amino acid residue at CDR L3 position 94 is aromatic (it is Tyr in J695); (iv) the amino acid residue at CDR L3 position 95A is drawn from the group of composed of Phe, Tyr, Trp, His, Asp, Glu, Asn, and Gln (it is His in J695); the amino acid residue at CDR L3 position 95B is Pro.

The amino acid residues of Site 5 form a β-hairpin loop that extends out from the center of the J695 combining site to contact IL-12 p40 residues Lys102, Asn103, and Lys104. Each of the above characteristics contributes either to the productive binding conformation of CDR L3 or to the binding specific interactions with IL-12. It is apparent from the J695/IL-12 p70 crystal structure determined here that CDR L3 variants in which one or more of the following changes, namely (i) CDR L3 length greater than 12 amino acid residues, (ii) substitution of a different aromatic residue for Tyr-L94, or (iii) substitution of a residue drawn from the group composed of Phe, Tyr, Trp, His, Asp, Glu, Asn, and Gln for His-L95A, could be made with retention or even enhancement of the binding characteristics of J695.

Site 6 comprises two residues drawn from the group composed of Tyr, Ser, Thr, Asn, Gln, Lys, and Arg, with both residues in CDR H2 (Arg-H52 and Tyr-H52A), such that the Cβ atoms of these two residues are separated by about 6 Å. The amino acid residues of J695 Site 6 form a wall against which IL-12 p40 residue Asp93 is placed; the two J695 amino acids form specific complementary charge and hydrogen bond interactions with the Asp93 side chain carboxylate. It is apparent from the J695/IL-12 p70 crystal structure determined here that one or more residues drawn from the group composed of Tyr, Ser, Thr, Asn, Gln, Lys, and Arg could be substituted for Arg-H52 or Tyr-H52A (e.g., corresponding to amino acid residues 52 or 53 of SEQ ID NO:1, respectively) with retention or even enhancement of the binding characteristics of J695.

Furthermore, it is apparent from the J695/IL-12 p70 crystal structure determined here that not all of the six Sites described above are needed to bind IL-12 p40 or other p40-containing cytokines. In particular, antibodies that possess at least one binding site drawn from the group composed of Site 1, Site 2, Site 3, Site 4, Site 5, and Site 6 described above, with variation of the sites as described above allowed, may exhibit retained or even enhanced binding characteristics compared to J695. Similarly, antibodies that possess two, three, four, five, or six binding sites drawn from the group of Sites 1 through 6 described above, with variation of the sites as described above allowed, may exhibit retained or even enhanced binding characteristics compared to J695.

Accordingly, in one aspect, the invention provides an antibody that binds to the p40 subunit of IL-12 and/or IL-23, wherein the antibody comprises the heavy chain variable region amino acid sequence of SEQ ID NO: 1 and the light chain variable region amino acid sequence of SEQ ID NO: 2, wherein any one of the variable region residues other than amino acid residues 27, 32, 52, 53, 97, 101 and 102 of SEQ ID NO: 1 and amino acid residues 35, 51 and 90-101 of SEQ ID NO: 2 are independently substituted with a different amino acid.

In another aspect, the invention provides an antibody that binds to the p40 subunit of IL-12 and/or IL-23, wherein the antibody comprises the heavy chain variable region amino acid sequence of SEQ ID NO: 1 and the light chain variable region amino acid sequence of SEQ ID NO: 2, wherein one or more of the variable region amino acid residues 27, 32, 52, 53, 97, 101 and 102 of SEQ ID NO: 1 and 35, 51 and 90-101 of SEQ ID NO: 2 are independently substituted with a different amino acid residue. In one embodiment of this aspect, one or more of the variable region amino acid residues 27, 32 and 102 of SEQ ID NO: 1 are independently substituted with an aromatic residue. In an additional embodiment, one or more of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln. In an additional embodiment, one or more of the variable region amino acid residues 92 and 97 of SEQ ID NO: 2 are independently substituted with an aromatic amino acid residue. In still another embodiment, one or more of the variable region amino acid residues 101 of SEQ ID NO: 1 and 51 of SEQ ID NO: 2 are independently substituted with an amino acid residue selected from the group consisting of Tyr, Ser, Thr, Asn and Gln. In a further embodiment, the variable region amino acid residue 91 of SEQ ID NO: 2 is independently substituted with any amino acid residue except Gln. In another embodiment, the variable region amino acid residue 95 of SEQ ID NO: 2 is independently substituted with a different aromatic amino acid residue. In still another embodiment, the variable region amino acid residue 97 of SEQ ID NO: 2 is substituted with an amino acid residue selected from the group consisting of Phe, Tyr, Trp, His, Asp, Glu, Asn and Gln. In yet another embodiment, one or more of the variable region amino acid residues 90-101 of SEQ ID NO: 2 is independently substituted with at least one or more different amino acids, and wherein the length of CDRL3 of the antibody is greater than or equal to 12 amino acid residues.

In an additional embodiment, the invention provides an antibody that binds to the p40 subunit of IL-12 and/or IL-23, wherein the antibody comprises the heavy chain variable region amino acid sequence of SEQ ID NO: 1 and the light chain variable region amino acid sequence of SEQ ID NO: 2, wherein the antibody has one or more of the following substitutions: (a) one or more of the variable region amino acid residues 90-101 of SEQ ID NO: 2 is independently substituted with at least one or more different amino acids, and wherein the length of CDRL3 of the antibody is greater than or equal to 12 amino acid residues; (b) variable region amino acid residue 91 of SEQ ID NO: 2 is substituted with any amino acid residue except Gln; (c) variable region amino acid residue 95 of SEQ ID NO: 2 is substituted with a different aromatic amino acid residue; or (d) variable region amino acid residue 97 of SEQ ID NO: 2 is substituted with an amino acid residue selected from the group consisting of Phe, Tyr, Trp, His, Asp, Glu, Asn and Gln. In another embodiment, one or more of the variable region amino acid residues 52 and 53 of SEQ ID NO: 1 is independently substituted with an amino acid residue selected from the group consisting of Tyr, Ser, Thr, Asn, Gln, Lys and Arg.

In a related aspect, the invention provides methods for altering the activity of an antibody that binds to the p40 subunit of IL-12 and/or IL-23, wherein the antibody comprises the heavy chain variable region amino acid sequence of SEQ ID NO: 1 and the light chain variable region amino acid sequence of SEQ ID NO: 2. In one embodiment of this aspect of the invention, the method comprises substituting one or more of the variable region amino acid residues 27, 32, 52, 53, 97, 101 and 102 of SEQ ID NO: 1 and amino acid residues 35, 51 and 90-101 of SEQ ID NO: 2 with a different amino acid residue, thereby altering the activity of an antibody that binds to the p40 subunit of IL-12 and/or IL-2. In an additional embodiment, one or more of the variable region amino acid residues 27, 32 and 102 of SEQ ID NO: 1 are independently substituted with an aromatic residue. In a further embodiment, one or more of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln. In still another embodiment, one or more of the variable region amino acid residues 92 and 97 of SEQ ID NO: 2 are independently substituted with an aromatic amino acid residue. In yet another embodiment, one or more of the variable region amino acid residues 101 of SEQ ID NO: 1 and 51 of SEQ ID NO: 2 are independently substituted with an amino acid residue selected from the group consisting of Tyr, Ser, Thr, Asn and Gln. In another embodiment, the variable region amino acid residue 91 of SEQ ID NO: 2 is substituted with any amino acid residue except Gln. In an additional embodiment, the variable region amino acid residue 95 of SEQ ID NO: 2 is substituted with a different aromatic amino acid residue. In another embodiment, the variable region amino acid residue 97 of SEQ ID NO: 2 is substituted with an amino acid residue selected from the group consisting of Phe, Tyr, Trp, His, Asp, Glu, Asn and Gln. In another embodiment, one or more of the variable region amino acid residues 90-101 of SEQ ID NO: 2 is independently substituted with at least one or more different amino acids, and wherein the length of CDRL3 of the antibody is greater than or equal to 12 amino acid residues.

In another embodiment, the invention provides methods for altering the activity of an antibody that binds to the p40 subunit of IL-12 and/or IL-23, wherein the antibody comprises the heavy chain variable region amino acid sequence of SEQ ID NO: 1 and the light chain variable region amino acid sequence of SEQ ID NO: 2, wherein the antibody has one or more of the following substitutions: (a) one or more of the variable region amino acid residues 90-101 of SEQ ID NO: 2 is independently substituted with at least one or more different amino acids, and wherein the length of CDRL3 of the antibody is greater than or equal to 12 amino acid residues; (b) variable region amino acid residue 91 of SEQ ID NO: 2 is substituted with any amino acid residue except Gln; (c) variable region amino acid residue 95 of SEQ ID NO: 2 is substituted with a different aromatic amino acid residue; or (d) variable region amino acid residue 97 of SEQ ID NO: 2 is substituted with an amino acid residue selected from the group consisting of Phe, Tyr, Trp, His, Asp, Glu, Asn and Gln. In another embodiment, one or more of the variable region amino acid residues 52 and 53 of SEQ ID NO: 1 is independently substituted with an amino acid residue selected from the group consisting of Tyr, Ser, Thr, Asn, Gln, Lys and Arg.

### B. Additional Useful Alterations to J695 Based Upon J695 Fab/IL-12 p70 Complex Structure

Although J695 makes a large number of specific interactions with IL-12, as described in detail above, additional changes to the J695 combining site would provide variant antibodies that may exhibit retained or even enhanced binding characteristics compared to J695. Notably, a large gap is present between J695 and IL-12 p40 at the combining site. Binding of p40 only partly fills the combining site's deep cleft, leaving an unfilled gap (FIG. 9, arrow), especially between J695 CDRs H2 and L3 and p40 Loops 3 and 4. Thus, variants that address this gap, or other deficiencies, would be beneficial. These antibody variants would be expected to exhibit improved characteristics by four mechanisms: (i) to make additional specific interactions with IL-12 p40; (ii) to fill gaps that exist between J695 and IL-12 p40; (iii) to limit the motion of IL-12 p40 once bound to a variant antibody combining site; or (iv) to pre-organize the variant antibody into the productive binding conformation. Some combination of these four mechanisms may also lead to more therapeutically effective antibodies. In particular, five groups of variations to the J695 amino acid sequence alone or in combination, may be performed as described below.

First, antibodies which possesses at least two of the binding sites selected from the group consisting of Site 1, Site 2, Site 3, Site 4, Site 5, and Site 6 described above, and which possesses in addition an amino acid residue at CDR H1 position 33 (e.g., corresponding to amino acid residue 33 of SEQ ID NO: 1) selected from the group consisting of Phe, Tyr, Trp, His, Met, Val, Leu, Ile, Pro, Ala, Ser, Thr, Asn, Gln, Arg, and Lys, may exhibit retained or even enhanced binding characteristics compared to J695. In particular, the mutation Gly-H33-Lys at this position would be expected to fill the gap between J695 and the IL-12 p40 amino acid residue Glu88, and LysH33 and Glu88 would be expected to make an additional salt-bridge interaction.

Second, antibodies which possesses at least two of the binding sites selected from the group consisting of Site 1, Site 2, Site 3, Site 4, Site 5, and Site 6 described above, and which possesses in addition an amino acid residue at CDR H2 position 50 (e.g., corresponding to amino acid residue 50 of SEQ ID NO: 1) selected from the group consisting of Phe, Tyr, Trp, His, Met, Gln, Arg, and Lys, may exhibit retained or even enhanced binding characteristics compared to J695. In particular, the mutations Phe-H50-Tyr and Phe-H50-Trp at this position would be expected to fill the gap between J695 and the IL-12 p40 amino acid residues Thr92 and Lys104.

Third, antibodies which possesses at least two of the binding sites selected from the group consisting of Site 1, Site 2, Site 3, Site 4, Site 5, and Site 6 described above, and which possesses in addition an amino acid residue at CDR H2 position 56 (e.g., corresponding to amino acid residue 57 of SEQ ID NO: 1) selected from the group consisting of Phe, Tyr, Trp, His, Met, Val, Leu, Ile, Pro, Ala, Ser, Thr, Asp, Glu, Asn, and Gln may exhibit retained or even enhanced binding characteristics compared to J695. In particular, the mutations Asn-H56-Ile and Asn-H56-Trp at this position would be expected to fill the gap between J695 and the IL-12 p40 amino acid residues Asp97 and Lys104, and to limit the motion of IL-12 p40 once bound to the antibody. Furthermore, the mutations Asn-H56-Ser and Asn-H56-Thr at this position would be expected in addition to pre-organize ArgH52 into the productive binding conformation by formation of a hydrogen bond between Ser Oγ (Oγ1 in Thr) and Arg Nε.

Fourth, antibodies which possesses at least two of the binding sites selected from the group consisting of Site 1, Site 2, Site 3, Site 4, Site 5, and Site 6 described above, and which possesses in addition an amino acid residue at CDR H3 position 95 (e.g., corresponding to amino acid residue 99 of SEQ ID NO: 1) selected from the group consisting of Phe, Tyr, Trp, His, Met, Arg, and Lys, may exhibit retained or even enhanced binding characteristics compared to J695. In particular, the mutations His-H95-Tyr and His-H95-Trp at this position would be expected to fill the gap between J695 and the IL-12 p40 amino acid residue Glu86, and to limit the motion of IL-12 p40 once bound to the antibody. Furthermore, the mutation His-H95-Tyr at this position would be expected in addition to form a hydrogen bond between Tyr O_{η} and the carbonyl oxygen atom of Glu86.

Fifth, antibodies which possesses at least two of the binding sites selected from the group consisting of Site 1, Site 2, Site 3, Site 4, Site 5, and Site 6 described above, and which possesses in addition an amino acid residue at CDR L1 position 32 (e.g., corresponding to amino acid residue 33 of SEQ ID NO: 2) selected from the group consisting of Phe, Tyr, Trp, His, Gln and Lys, may exhibit retained or even enhanced binding characteristics compared to J695. In particular, the mutations Thr-L32-Tyr and Thr-L32-Trp at this position would be expected to fill the gap between J695 and the IL-12 p40 amino acid residue Gly88.

Accordingly, the present invention also provides, in one aspect, an antibody that binds to the p40 subunit of IL-12 and/or IL-23, wherein the antibody comprises the heavy chain variable region amino acid sequence of SEQ ID NO: 1 and the light chain variable region amino acid sequence of SEQ ID NO: 2, wherein one or more of the variable region amino acid residues 33, 50, 57 and 99 of SEQ ID NO: 1 and 33 of SEQ ID NO: 2 are independently substituted with a different amino acid residue. In one embodiment, the variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys. In another embodiment, the variable region amino acid residue 33 of SEQ ID NO:1 is substituted with Lys. In a further embodiment, the variable region amino acid residue 50 of SEQ ID NO: 1 is substituted with an amino acid residue selected from the group consisting of Phe, Tyr, Trp, His, Met, Gln, Arg and Lys. In yet another embodiment, the variable region amino acid residue 50 of SEQ ID NO: 1 is substituted with Tyr or Trp.

In another embodiment, the variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln. In another embodiment, the variable region amino acid residue 57 of SEQ ID NO: 1 is substituted with Ile or Trp. In still another embodiment, the variable region amino acid residue 57 of SEQ ID NO: 1 is substituted with Ser or Thr. In a further embodiment, the variable region amino acid residue 99 of SEQ ID NO: 1 is substituted with an amino acid residue selected from the group consisting of Phe, Tyr, Trp, His, Met, Arg and Lys. In another embodiment, the variable region amino acid residue 99 of SEQ ID NO: 1 is substituted with Tyr or Trp. In an additional embodiment, the variable region amino acid residue 33 of SEQ ID NO: 2 is substituted with an amino acid residue selected from the group consisting of Phe, Tyr, Trp, His, Gln and Lys. In a further embodiment, the variable region amino acid residue 33 of SEQ ID NO: 2 is substituted with Tyr or Trp.

In another aspect, the invention provides antibodies that are capable of undergoing competitive binding; i.e., competitively inhibiting any of the antibodies described herein. Accordingly, in another embodiment the invention comprises an antibody that competes for binding of the p40 subunit of IL-12 and/or IL-23 with any of the antibody species described herein.

In another aspect, the invention provides methods for altering the activity of an antibody that binds to the p40 subunit of IL-12 and/or IL-23, wherein the antibody comprises the heavy chain variable region amino acid sequence of SEQ ID NO: 1 and the light chain variable region amino acid sequence of SEQ ID NO: 2, comprising substituting one or more of the variable region amino acid residues 33, 50, 57 and 99 of SEQ ID NO: 1 and 33 of SEQ ID NO: 2 with a different amino acid residue, thereby altering the activity of an antibody that binds to the p40 subunit of IL-12 and/or IL-23. In one embodiment of the method, the variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys. In another embodiment, the variable region amino acid residue 33 of SEQ ID NO: 1 is substituted with Lys. In an additional embodiment, the variable region amino acid residue 50 of SEQ ID NO: 1 is substituted with an amino acid residue selected from the group consisting of Phe, Tyr, Trp, His, Met, Gln, Arg and Lys. In a further embodiment, the variable region amino acid residue 50 of SEQ ID NO: 1 is substituted with Tyr or Trp. In another embodiment, the variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln. In an additional embodiment, the variable region amino acid residue 57 of SEQ ID NO: 1 is substituted with Ile or Trp. In yet another embodiment, the variable region amino acid residue 57 of SEQ ID NO: 1 is substituted with Ser or Thr. In still another embodiment, the variable region amino acid residue 99 of SEQ ID NO: 1 is substituted with an amino acid residue selected from the group consisting of Phe, Tyr, Trp, His, Met, Arg and Lys. In another embodiment, the variable region amino acid residue 99 of SEQ ID NO: 1 is substituted with Tyr or Trp. In a further embodiment, the variable region amino acid residue 33 of SEQ ID NO: 2 is substituted with an amino acid residue selected from the group consisting of Phe, Tyr, Trp, His, Gln and Lys. In still another embodiment, the variable region amino acid residue 33 of SEQ ID NO: 2 is substituted with Tyr or Trp.

In a further aspect, the invention provides and encompasses an antibody as described herein, including an antibody produced according to any of the methods described herein. For example, in any of the antibody embodiments described herein, the antibody binds to the p40 subunit of IL-12 and/or IL-23 with a K_{off} of 1×10⁻³ M⁻¹ or less or a IQ of 1×10⁻¹⁰ M or less. Further, in any of the antibody embodiments encompassed by the invention, the antibody neutralizes the biological activity of the p40 subunit of IL-12 and/or IL-23. Functional characteristics of the antibodies encompassed by the invention are further discussed below in section V(C).

In a still further aspect, the antibodies of the invention are not one of the antibodies existing in the art and inherently binding to the epitopes identified in the specification herein. For example, in one embodiment, the antibodies of the invention are not an antibody described in U.S. Pat. No. 6,914,128, e.g., are not the antibody Y61 or J695 (as described in U.S. Pat. No. 6,914,129, the entire contents of which are hereby incorporated herein).

### C. Antibodies Based Upon the Determination of the Epitopes of Other Anti-IL-12 Antibodies

The epitopes of other anti-IL-12 antibodies were determined using a rat/human IL-12 p40 chimeric protein (or “chimeras”) approach. Predominantly human IL-12 p40 molecules that had certain rat IL-12 p40 amino acid residue(s) incorporated at specific positions were expressed and purified. Binding of these chimeras, as well as IL-12 control proteins (e.g., human and rat IL-12 p40 and/or p70), to a panel of antibodies (e.g., J695, C8.6.2 or C11.5.14, as described further below) was determined using surface plasmon resonance binding analysis. In addition, predominantly rat IL-12 p40 chimeras that had certain human IL-12 p40 amino acid residue(s) incorporated at specific positions were similarly expressed, purified, and analyzed.

1. Preparation of Human/Rat and Rat/Human IL-12 p40 Chimeras

The specific amino acid residues that were mutated in the IL-12 p40 chimeras are found in several different Sites located within IL-12 p40. The human/rat IL-12 p40 chimeras that were tested are listed in Table 1 and the rat/human IL-12 p40 chimeras are listed in Table 2.

TABLE 1
Predominantly human IL-12 p40 chimeras prepared
and tested for antibody binding.

| Human | Residues Mutated to the | |
|---|---|---|
| Chimera | Rat p40 Sequence | Site(s) |
| 1 | Y16R | 7a |
| 2 | D87N | 7b |
| 3 | D93E | 7c |
| 4 | D87N & D93E | 7b, 7c |
| 5 | D87N & P101F | 7b, 11 |
| 6 | 40-47 | 8 |
| 7 | 40-47 & 97-101 | 8, 11 |
| 8 | 97-101 | 11 |
| 9 | G35D & G61L | 9, 10 |
| 10 | 157-164 | 12 |
| 11 | None (control) | N/A |

TABLE 2
Predominantly rat IL-12 p40 chimeras prepared
and tested for antibody binding.

| Rat | Residues Mutated to the | |
|---|---|---|
| Chimera | Human p40 Sequence | Site(s) |
| A | R16Y | 7a |
| B | N87D | 7b |
| C | E93D | 7c |
| D | R16Y, N87D, E93D | 7 |

The cloning and construction of expression plasmids for preparing the chimeras were carried out as follows. The cDNA encoding the human IL-12p40 (purchased from InvivoGen, CA, catalog no. porf-hil12) subunit was PCR amplified by the Expand Polymerase Kit (Roche) using primers 5′-CAC CAT GGG TCA CCA GCA GTT GGT C-3′ (SEQ ID NO:7) and 5′-ACC CTG GAA GTA CAG GTT TTC ACT GCA GGG CAC AGA TGC CCA TTC GC-3′ (SEQ ID NO:8). The resulting 1009 bp product was cloned into pENTR/D-TOPO using the Gateway BP reaction (Invitrogen). Site-directed mutagenesis was performed using the Quick-Change XL Site-Directed Mutagenesis Kit according to manufacturer's instructions using plasmid pENTR/D-hIL-12p40 as a template and the oligonucleotide primers listed in Table 2.1. The presence of the desired mutations was confirmed by DNA sequencing. Following mutagenesis, wild type hIL-12p40 and mutants were subcloned into the mammalian expression vector pcDNA DEST40 using the Gateway LR reaction to make pcDNA DEST40-hIL-12p40 and variants thereof.

IL-12p40 chimeric proteins were expressed by transient transfection in HEK293.F cells. HEK293.F cells were cultured in 250 mL Erlenmeyer flasks (Corning, N.Y.) in Freestyle 293 expression medium (Invitrogen) at 8% CO₂ and 37° C. For each construct, 30×10⁶ cells were transfected with 30 μg of plasmid DNA using 293fectin in a 100 mL Erlenmeyer flask at 30 mL scale. Cells were incubated at 37° C., in a humidified 8% CO₂ atmosphere with shaking. After 72 hr, cells were harvested and supernatants analyzed for secreted IL-12p40 by Western blot. The hIL-12p40 containing supernatants were used directly in subsequent binding assays described below.

TABLE 2.1
List of Primers: Forward primers (F), and reverse primers (R)

| Primers | | SEQ ID |
|---|---|---|
| Name | Sequence | NO: |
| Ch1 (F) | 5′-CGTAGAATTGGATTGGCGTCCGGATGCCCCTGGAG-3′ | 9 |
| Ch1 (R) | 5′-CTCCAGGGGCATCCGGACGCCAATCCAATTCTACG-3′ | 10 |
| Ch2 (F) | 5′-CTGCTTCACAAAAAGGAAAACGGAATTTGGTCCACTG-3′ | 11 |
| Ch2 (R) | 5′-CAGTGGACCAAATTCCGTTTTCCTTTTTGTGAAGCAG | 12 |
| Ch3 (F) | 5′-GATGGAATTTGGTCCACTGAGATTTTAAAGGACCAGAAAG-3′ | 13 |
| Ch3 (R) | 5′-CTTTCTGGTCCTTTAAAATCTCAGTGGACCAAATTCCATC-3′ | 14 |
| Ch4 (F) | 5′-GGTCCACTGATATTTTAAAGAACCAGAAAGAATTCAAAAATAAGACCTTTCTAAGATG-3′ | 15 |
| Ch4 (R | 5′-CATCTTAGAAAGGTCTTATTTTTGAATTCTTTCTGGTTCTTTAAAATATCAGTGGACC-3′ | 16 |
| Ch5 (F) | 5′-GACACCCCTGAAGAAGATGACATCACCTGGACCTTGGACC-3′ | 17 |
| Ch5 (R | 5′ GGTCCAAGGTCCAGGTGATGTCATCTTCTTCAGGGGTGTC-3′ | 18 |
| Ch6 (F) | 5′-GATGGTATCACCTGGACCTCCGACCAGCGCCGGGGGGTCATCGGCTCTGGCAAAACCCTG-3′ | 19 |
| Ch6 (R) | 5′-GGTCAGGGTTTTGCCAGAGCCGATGACCCCCCGGCGCTGGTCGGAGGTCCAGGTGATACC-3 | 20 |
| Ch7 (F) | Primers sets 6 & 9 | |
| Ch8 (F) | 5′-GCTGCTACACTCTCTGCAGAGAAGGTCACCCTGAACCAGCGTGACTATGAGTACTC-3′ | 21 |
| Ch8 (R) | 5′-GGCACTCCACTGAGTACTCATAGCACGCTGGTTCAGGGTGACCTTCTCTGCAGA-3′ | 22 |
| Ch9 (F) | 5′-GGTCCACTGATATTTTAAAGAACTTCAAAAATAAGACCTTTCTAAGATG-3′ | 23 |
| Ch9 (R | 5′-CATCTTAGAAAGGTCTTATTTTTGAAGTTCTTTAAAATATCAGTGGACC-3′ | 24 |
| Ch10 (F) | 5′-GTCCACTGATATTTTAAAGGACCCCAAAAATAAGACCTTTCTAAG-3′ | 25 |
| Ch10 (R | 5′-CTTAGAAAGGTCTTATTTTTGGGGTCCTTTAAAATATCAGTGGAC-3′ | 26 |

2. The Human/Rat and Rat/Human Chimeras Define Seven Additional Sites on IL-12 p40

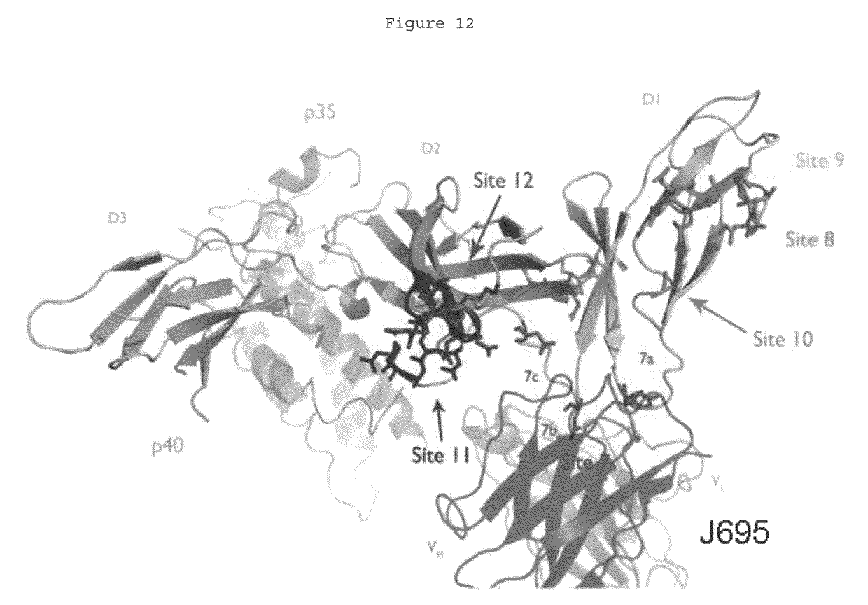

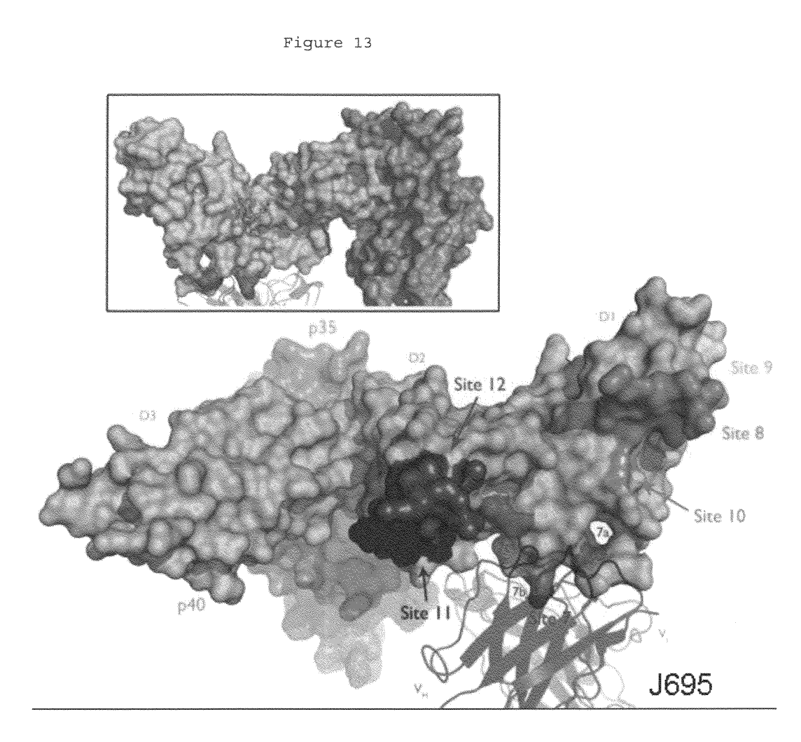

Seven additional “Sites” defined and delineated by the Il-12 p40 chimeras are shown in relationship to an alignment of several IL-12 p40 amino acid sequences in FIG. 11, and relative to the three-dimensional structure of IL-12 p70 (and bound J695) in FIGS. 6, 12 and 13. These Sites are described in more detail below, and are summarized in Table 3 below.

Site 7 comprises human IL-12 p40 amino acid residues Tyr16, Asp87, and Asp93. These residues are located on two different surface loops on domain 1 of IL-12 p40 (Yoon, C., S. C. Johnston, et al. (2000). “Charged residues dominate a unique interlocking topography in the heterodimeric cytokine interleukin-12.” The EMBO Journal 19 (14): 3530-3521). Taken alone, the residues of Site 7 define a discontinuous (or conformational) epitope, as revealed by the J695/IL-12 p70 complex crystal structure. Site 7 can be considered to consist of three sub-Sites, namely sub-Site 7a (Tyr16), sub-Site 7b (Asp87), and sub-Site 7c (Asp93).

Site 8 comprises human IL-12 p40 amino acid residues Leu40, Asp41, Gln42, Ser43, Ser44, Glu45, Val46, and Leu47. These residues form a surface loop on domain 1 of IL-12 p40 (Yoon, C., S. C. Johnston, et al. (2000). “Charged residues dominate a unique interlocking topography in the heterodimeric cytokine interleukin-12.” The EMBO Journal 19 (14): 3530-3521). Taken alone, the residues of Site 8 define a continuous (or linear) epitope.

Site 9 comprises human IL-12 p40 amino acid residue Gly35. This residue is located on a surface loop on domain 1 of IL-12 p40 (Yoon, C., S. C. Johnston, et al. (2000). “Charged residues dominate a unique interlocking topography in the heterodimeric cytokine interleukin-12.” The EMBO Journal 19 (14): 3530-3521) positioned on one side of the Site 8 loop (on the side opposite Site 10; see below). Taken alone, the residue of Site 9 defines a continuous (or linear) epitope.

Site 10 comprises human IL-12 p40 amino acid residue Gly61. This residue is located on a surface loop on domain 1 of IL-12 p40 (Yoon, C., S. C. Johnston, et al. (2000). “Charged residues dominate a unique interlocking topography in the heterodimeric cytokine interleukin-12.” The EMBO Journal 19 (14): 3530-3521) positioned on one side of the Site 8 loop (on the side opposite Site 9; see above). Taken alone, the residue of Site 10 defines a continuous (or linear) epitope.

Site 11 comprises human IL-12 p40 amino acid residues Asp97, Gln98, Lys99, Glu100, and Pro101. These residues form a surface loop on domain 1 of IL-12 p40 (Yoon, C., S. C. Johnston, et al. (2000). “Charged residues dominate a unique interlocking topography in the heterodimeric cytokine interleukin-12.” The EMBO Journal 19 (14): 3530-3521). Taken alone, the residues of Site 11 define a continuous (or linear) epitope.

Site 12 comprises human IL-12 p40 amino acid residues Arg157, Val158, Arg159, Gly160, Asp161, Asn162, Lys163, and Glu164. These residues form a (disordered) surface loop on domain 2 of IL-12 p40 (Yoon, C., S. C. Johnston, et al. (2000). “Charged residues dominate a unique interlocking topography in the heterodimeric cytokine interleukin-12.” The EMBO Journal 19 (14): 3530-3521); this loop is ordered in the J695 Fab/IL-12 p70 complex structure described here. Taken alone, the residues of Site 12 define a continuous (or linear) epitope.

TABLE 3
Summary of Sites 7-12

| Site | Amino Acid Residues |
|---|---|
| 7 | Tyr16 (7a), Asp87 (7b), Asp93 (7c) |
| 8 | Leu40, Asp41, Gln42, Ser43, Ser44, Glu45, |
| | Val46, Leu47 |
| 9 | Gly35 |
| 10 | Gly61 |
| 11 | Asp97, Gln98, Lys99, Glu100, Pro101 |
| 12 | Arg157, Val158, Arg159, Gly160, Asp161, |
| | Asn162, Lys163, Glu164 |

The binding of the rat/human IL-12 p40 chimeras by various antibodies was analyzed by Surface Plasmon Resonance. Specifically, antibody was covalently linked via free amine groups to the Biacore chip dextran matrix by first activating carboxyl groups on the matrix with 100 mM N-hydroxysuccinimide (NHS) and 400 mM N-Ethyl-N′-(3-dimethylaminopropyl)-carbodiimide hydrochloride (EDC). This was completed across four different flow cells. Approximately fifty microliters of each antibody (25 μg/mL) diluted in sodium acetate, pH 4.5, was injected across the activated biosensor and free amines on the protein were bound directly to the activated carboxyl groups. Typically, 5000 resonance units were immobilized. Unreacted matrix EDC-esters were deactivated by an injection of 1 M ethanolamine.

To ascertain the epitope pattern of several different monoclonal antibodies against IL-12p40 supernatant samples, a direct binding assay was conducted. Aliquots of recombinant human IL-12p40 (100 nM) were injected across covalently immobilized antibody on the Biacore dextran chip biosensor surface at a flow rate of 25 mL/min. Before injection of the antigen and immediately afterward, HBS-EP buffer alone flowed through each flow cell. The net difference in the signals between the baseline and the point corresponding to approximately 30 seconds after completion of ligand injection was taken to represent the final binding value (approximately 500-2500 RU's). The response was measured in Resonance Units (RU's). A positive pair-wise binding sensorgram was declared only where binding of the first probe to the target molecule was rapid and strong. The covalently immobilized antibody-coupled surfaces were completely regenerated using 10 mM HCl (5 min contact time) and retained their full binding capacity over twenty cycles.

A summary of the binding data obtained by Surface Plasmon Resonance for the human/rat and rat/human IL-12 p40 chimeras is summarized below in Table 3.1.

TABLE 3.1
Summary of surface plasmon resonance binding data obtained with the human IL-12
p40 chimeras that possess mutations to the corresponding rat p40 residues.

| | Human Chimera* | | | | | | | | | |
|---|---|---|---|---|---|---|---|---|---|---|
| | Site(s) | | | | | | | | | |
| | | | | 4 | 5 | | 7 | | 9 | |
| | 1 | 2 | 3 | 7b, 7c | 7c, 11 | 6 | 8, 11 | 8 | 9, 10 | 10 |
| | 7a | 7b | 7c | D87N & | D87N & | 8 | 40-47 & | 11 | G35D & | 12 |
| mAb | Y16R | D87N | D93E | D93E | P101F | 40-47 | 97-101 | 97-101 | G61L | 157-164 |
| J695 | ++ | ++ | ± | ND | − | − | − | − | − | − |
| 1A6.1 | − | − | ± | ND | − | − | − | − | + | − |
| 1D4.1 | − | − | − | − | − | + | + | − | + | − |
| 1D4.7 | − | − | − | − | − | + | + | − | ± | − |
| 3G7.2 | − | − | − | ND | − | + | + | − | + | − |
| 8E1.1 | + | + | ± | ND | + | − | + | + | − | − |
| C8.6.2 | − | − | − | − | − | + | + | − | ± | − |
| C11.5.14 | − | − | − | ND | − | − | ± | ± | − | ++ |

*Chimeras are listed in Table 3. Data are summarized as: “ND”, no data were measured; “−”, no effect; “±”, weak effect (slightly faster k_{off}); “+”, strong effect (much faster k_{off}); “++”, extremely strong effect (no significant binding was observed);

3. Delineation and Definition of Seven Additional IL-12 p40 Epitopes as Determined by Binding Analysis of Human/Rat IL-12 p40 Chimeras

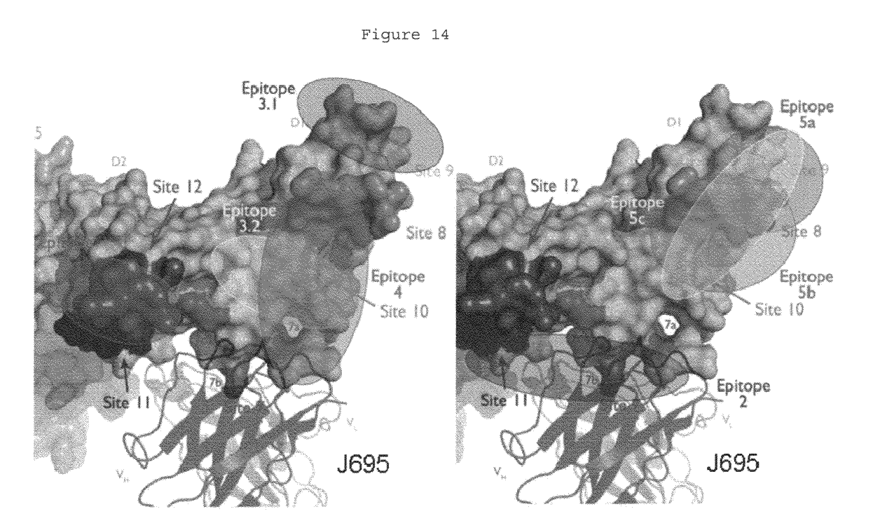

Using the chimeras and surface plasmon resonance methodology described above, seven additional Epitopes of IL-12 p40, in addition to the crystallographically-determined J695 epitope (e.g., as described above in sections II-V), were delineated and defined. Epitope 1 identified using the chimeras and surface plasmon resonance methodology comprises amino acid residues falling within the crystallographically-determined J695 epitope, and thereby confirms the crystallographically-determined J695 epitope. The antibody/chimera binding data are summarized above in Table 3.1. These Epitopes comprise one or more antigenic “Sites”, described above, on the surface of IL-12 p40. These Sites are shown in relationship to an alignment of several IL-12 p40 amino acid sequences in FIG. 11, and relative to the three-dimensional structure of IL-12 p70 (and bound J695) in FIGS. 6, 12 and 13. The additional six Epitopes, namely Epitopes 2, 3.1 or 3.2, 4a, 4b, 4c, and 5, are illustrated schematically in FIG. 14. All eight Epitopes, i.e., Epitopes 1-5 (i.e., Epitopes 1, 2, 3.1 or 3.2, 4a, 4b, 4c and 5) are summarized in Table 4 and are described in detail below.

TABLE 4
Summary of antibody Epitopes determined by surface plasmon resonance
binding data obtained with the human IL-12 p40 chimeras that possess
mutations to the corresponding rat p40 residues.

| | | Major | Minor | |
|---|---|---|---|---|
| mAb | Epitope | Site(s) | Site(s) | Comments |
| J695 | 1 | 7a | 7c | In accord with crystallographically-determined |
| | | 7b | | epitope |
| 1A6.1 | 3 | 9 or 10 | 7c | Binding to both Sites 9 and 10 not consistent with |
| | (3.1, 3.2) | | | lack of effect of Site 8 |
| 1D4.1 | 4 (a, b, c) | 8 | — | Since Site 8 is flanked by Sites 9 and 10, binding |
| | | 9 and/or 10 | | could be to Sites 8 and 9, 8 and 10, or 8, 9, and 10 |
| 1D4.7 | 4 (a, b, c) | 8 | — | Since Site 8 is flanked by Sites 9 and 10, binding |
| | | 9 and/or 10 | | could be to Sites 8 and 9, 8 and 10, or 8, 9, and 10 |
| 3G7.2 | 4 (a, b, c) | 8 | — | Since Site 8 is flanked by Sites 9 and 10, binding |
| | | 9 and/or 10 | | could be to Sites 8 and 9, 8 and 10, or 8, 9, and 10 |
| 8E1.1 | 2 | 7a | 7c | Related to Epitope 1, but distinct due to effect from |
| | | 7b | | Site 11 |
| | | 11 | | |
| C8.6.2 | 4 (a, b, c) | 8 | | Since Site 8 is flanked by Sites 9 and 10, binding |
| | | 9 and/or 10 | | could be to Sites 8 and 9, 8 and 10, or 8, 9, and 10 |
| C11.5.14 | 5 | 12 | — | In accord with FLITRX-determined epitope |

Epitope 1. Antibodies that bind to IL-12 p40 at Epitope 1 include: J695 (as described in PCT Publication No. WO0056772 A1). Mutation at Sites 7a (Tyr16) and 7b (Asp87) ablates binding; mutation at Site 7c (Asp93) has a minor effect. This biochemically-defined epitope is consistent with that observed crystallographically.

Epitope 2. Antibodies that bind to IL-12 p40 at Epitope 2 include: the humanized monoclonal antibody 8E1.1. A description of antibody 8E11.1 can be found at least in U.S. Pat. No. 7,700,739, the entire contents of which, and in particular the description of antibody 8E11.1, are hereby incorporated herein. Mutation at Sites 7a (Tyr16), 7b (Asp87), and 11 (Asp97, Gln98, Lys99, Glu100, and Pro101) has a strong effect on binding; mutation at Site 7c (Asp93) has a minor effect. Epitope 2 is clearly related to Epitope 1, but the strong effect of mutation at Site 11 upon the binding of 8E1.1, but not that of J695, distinguishes these two Epitopes.

Epitope 3. Antibodies that bind to IL-12 p40 at Epitope 3 include: the humanized monoclonal antibody 1A6.1 A description of antibody 1A6.1 can be found at least in U.S. Pat. No. 7,700,739, the entire contents of which, and in particular the description of antibody 1A6.1, are hereby incorporated herein. Mutation at Sites 9 (Gly35) and 10 (Gly61) together had a strong effect upon binding. These two residues were only mutated together. Alone, it would be impossible to determine whether Epitope 3 is defined by one glycine, or the other, or both. But, the complete lack of effect of mutation at Site 8 (Leu40, Asp41, Gln42, Ser43, Ser44, Glu45, Val46, and Leu47), coupled with knowledge of the three-dimensional structure of IL-12 p40, indicates that Epitope 3 is defined by binding either to Site 9 and, given the minimal size of antibody combining sites (Davies, D. R., E. A. Padlan, et al. 1990 “Antibody-antigen complexes.” Annu Rev Biochem 59: 439-73; Davies, D. R. and G. H. Cohen 1996 “Interactions of protein antigens with antibodies.” Proc Natl Acad Sci USA 93 (1): 7-12), other residues surrounding Site 9 (Gly35) that are distal to Site 8, i.e. Epitope 3.1, or to Site 10 (Gly61) and other residues surrounding Site 10 that are distal to Site 8, i.e. Epitope 3.2, but not both. The true Epitope 3 is one or the other of 3.1 and 3.2, but not both.

Epitope 4. Antibodies that bind to IL-12 p40 at Epitope 4 include the reference murine antibody C8.6.2 (D'Andrea, A., M. Rengaraju, et al. (1992). “Production of natural killer cell stimulatory factor (interleukin 12) by peripheral blood mononuclear cells.” J. Exp. Med. 176: 1387-1398), and three humanized monoclonal antibodies, namely 3G7.2, 1D4.1, and 1D4.7. A description of antibodies 3G7.2, 1D4.1, and 1D4.7 can be found at least in U.S. Pat. No. 7,700,739, the entire contents of which, and in particular the description of antibodies 3G7.2, 1D4.1, and 1D4.7, are hereby incorporated herein. Mutation at Site 8 (Leu40, Asp41, Gln42, Ser43, Ser44, Glu45, Val46, and Leu47) strongly affected binding, and mutation at either Site 9 (Gly35) or Site 10 (Gly61) had a weak or strong effect. Again drawing on knowledge of the three-dimensional structure of IL-12 p40, since Site 8 is flanked by Sites 9 and 10, binding of any of these antibodies could be to Sites 8 and 9, Sites 8 and 10, or Sites 8, 9, and 10. Thus, Epitope 4 actually defines a family of related, partially overlapping epitopes, namely: Epitope 4a (Sites 8 and 9); Epitope 4b (Sites 8 and 10); and Epitope 4c (Sites 8, 9, and 10). Antibodies C8.6.2, 3G7.2, 1D4.1, and 1D4.7 could each bind to any epitope taken from the list of Epitopes 4a, 4b, and 4c; they are under no constraint to bind to the same epitope.

Epitope 5. Antibodies that bind to IL-12 p40 at Epitope 5 include the reference murine antibody C11.5.14 (D'Andrea, A., M. Rengaraju, et al. (1992). “Production of natural killer cell stimulatory factor (interleukin 12) by peripheral blood mononuclear cells.” J. Exp. Med. 176: 1387-1398). Mutation at Site 12 (Arg157, Val158, Arg159, Gly160, Asp161, Asn162, Lys163, and Glu164) ablated binding of C11.5.14, and mutation at Site 11 had a weak effect (Asp97, Gln98, Lys99, Glu100, and Pro101). These chimera-derived binding results that define Epitope 5 are consistent with the previously-determined C11.5.14 epitope determined by “FLITRX” peptide display on thioredoxin/flagellin fusion proteins (Lu, Z., K. S. Murray, et al. (1995). “Expression of thioredoxin random peptide libraries on the Escherichia coli cell surface as functional fusions to flagellin: a system designed for exploring protein-protein interactions.” Biotechnology (N Y) 13 (4): 366-72).

Accordingly, in an additional aspect, the invention provides an antibody that binds to the p40 subunit of IL-12 and/or IL-23, wherein the antibody binds to a conformational epitope. In one embodiment, the conformational epitope comprises at least one amino acid residue selected from the group consisting of amino acid residues 16, 87 and 93 of the amino acid sequence of SEQ ID NO:3 (e.g., Epitope 1, comprising Sites 7a-c). In another embodiment, the antibody binds to amino acid residue 16 (i.e., Site 7a). It is to be understood that, in certain embodiments, when reference is made to an antibody of the invention binding an epitope, e.g., a conformational epitope, the intention is for the antibody to bind only to those specific residues that make up the epitope and not other residues in the linear amino acid sequence of the antigen, e.g., the p40 subunit of IL-12 and/or IL-23.

In another aspect, the invention provides an antibody that binds to the p40 subunit of IL-12 and/or IL-23, wherein the antibody binds to a conformational epitope comprising at least one amino acid residue selected from the group consisting of amino acid residues 16, 87 and 93 of the amino acid sequence of SEQ ID NO:3 (e.g., Epitope 1, comprising Sites 7a-c) and any epitope described in US 2009/0202549, the entire contents of which are hereby incorporated by reference herein.

In an additional aspect; the invention provides an antibody that binds to the p40 subunit of IL-12 and/or IL-23, wherein the antibody binds to a conformational epitope comprising at least one amino acid residue selected from the group consisting of amino acid residues 97, 98, 99, 100 and 101 of SEQ ID NO:3 (e.g., Epitope 2, comprising Sites 7a, 7b and 11). In another aspect, the invention provides an antibody that binds to the p40 subunit of IL-12 and/or IL-23, wherein the antibody binds to a conformational epitope comprising at least one amino acid residue selected from the group consisting of amino acid residues 16, 87, 93, 97, 98, 99, 100 and 101 of SEQ ID NO:3 (e.g., Epitope 2, comprising Sites 7a, 7b and 11 and 7c).

In an additional aspect, the invention provides an antibody that binds to the p40 subunit of IL-12 and/or IL-23, wherein the antibody binds to a conformational epitope comprising at least one amino acid residue selected from the group consisting of amino acid residues 35 and 36 of SEQ ID NO:3 (e.g., Epitope 3, comprising Sites 9 or 10). In one embodiment, the antibody binds to the p40 subunit of IL-12 and/or IL-23, wherein the antibody binds to a conformational epitope comprising amino acid residue 35 or amino acid residue 36 of SEQ ID NO:3 (e.g., Epitope 3, comprising Sites 9 or 10). In a related aspect, the invention provides an antibody that binds to the p40 subunit of IL-12 and/or IL-23, wherein the antibody binds to a conformational epitope comprising amino acid residue 93 and further comprising amino acid residue 35 or amino acid residue 36 of SEQ ID NO:3 (e.g., Epitope 3, comprising Sites 9 or 10, and 7c).

In an additional aspect, the invention provides an antibody that binds to the p40 subunit of IL-12 and/or IL-23, wherein the antibody binds to a conformational epitope comprising at least one amino acid residue selected from the group consisting of amino acid residues 40-47 and 35 of SEQ ID NO:3 (e.g., Epitope 4a, comprising Sites 8 and 9). In an related aspect, the invention provides an antibody that binds to the p40 subunit of IL-12 and/or IL-23, wherein the antibody binds to a conformational epitope comprising at least one amino acid residue selected from the group consisting of amino acid residues 40-47 and 61 of SEQ ID NO:3 (e.g., Epitope 4b, comprising Sites 8 and 10). In a further related aspect, the invention provides an antibody that binds to the p40 subunit of IL-12 and/or IL-23, wherein the antibody binds to a conformational epitope comprising at least one amino acid residue selected from the group consisting of amino acid residues 40-47, 35 and 62 of SEQ ID NO:3 (e.g., Epitope 4c, comprising Sites 8, 9 and 10).

In an additional aspect, the invention provides an antibody that binds to the p40 subunit of IL-12 and/or IL-23, wherein the antibody binds to a conformational epitope comprising at least one amino acid residue selected from the group consisting of amino acid residues 157-164 of SEQ ID NO:3 (e.g., Epitope 5, comprising Site 12).

In one embodiment, the antibody does not bind to one or more of: (a) a conformational epitope comprising at least one amino acid residue selected from the group consisting of residues 16, 87 and 97-101 of the amino acid sequence of SEQ ID NO:3 (e.g., Epitope 2, comprising Sites 7a, 7b and 11); (b) a conformational epitope comprising at least one amino acid residue selected from the group consisting of residues 35 and 61 of the amino acid sequence of SEQ ID NO:3 (e.g., Epitope 3, comprising Sites 9 or 10); (c) a conformational epitope comprising at least one amino acid residue selected from the group consisting of residues 40-47, 35 and 61 of the amino acid sequence of SEQ ID NO:3 (e.g, Epitopes 4a-c, comprising Sites 8, 9 and/or 10); and (c) a continuous epitope comprising at least one amino acid residue selected from the group consisting of residues 157-164 of the amino acid sequence of SEQ ID NO:3 (e.g., Epitope 5, comprising Site 12).

4. Description of Additional IL-12 p40 Binding Sites as Determined by Binding Analysis of Human/Rat IL-12 p40 Chimeras Combined with Knowledge of the J695 Fab/IL-12 p70 Crystal Structure.

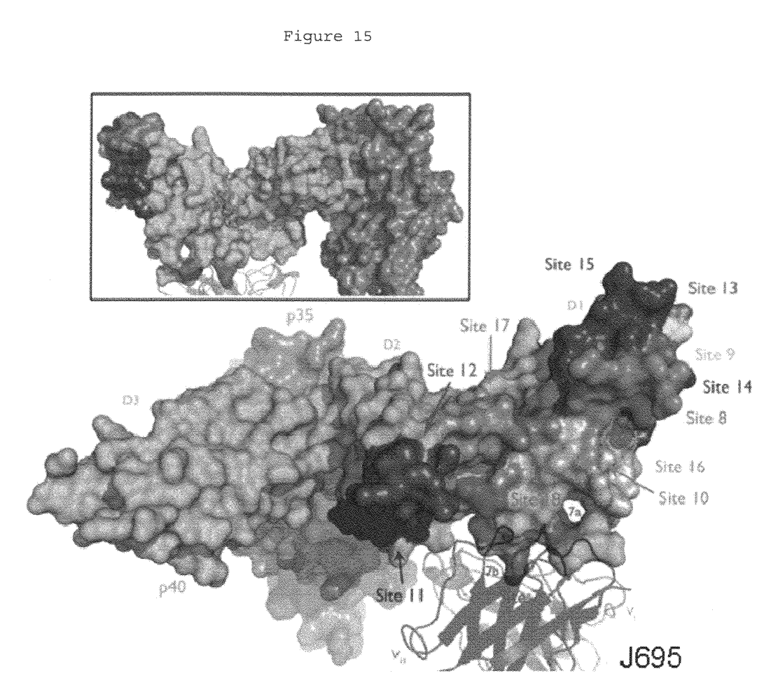

Additional binding sites can be determined from the surface plasmon resonance binding data obtained with human/rat IL-12 p40 chimeras, described above, combined with knowledge of the three-dimensional disposition of these sites, as provided by the J695 Fab/IL-12 p70 crystal structure. These additional antibody binding Sites are shown in FIG. 15.

For example, as discussed above in reference to Epitopes 3.1 and 3.2, the humanized monoclonal antibody 1A6.1 binds either to Site 9 (Gly35) or to Site 10 (Gly61), but not to both simultaneously, because simultaneous binding would be inconsistent with the complete lack of effect of mutation at Site 8 (Leu40, Asp41, Gln42, Ser43, Ser44, Glu45, Val46, and Leu47) upon the binding, given the known sizes and shapes of antibody combining sites (Davies, D. R., E. A. Padlan, et al. (1990). “Antibody-antigen complexes.” Annu Rev Biochem 59: 439-73; Davies, D. R. and G. H. Cohen (1996). “Interactions of protein antigens with antibodies.” Proc Natl Acad Sci USA 93 (1): 7-12).

Therefore, antibody 1A6.1 either binds to Site 9 and in addition other residues surrounding Site 9 (Gly35) that are distal to Site 8, i.e. Epitope 3.1; or, antibody 1A6.1 binds to Site 10 and in addition other residues surrounding Site 10 (Gly61) that are distal to Site 8, i.e. Epitope 3.2. These “other residues”, which are mostly located on surface-exposed loops of IL-12 p40, are defined below:

Site 13, which is located near Site 9 but is distal to Site 8, comprises IL-12 p40 amino acid residues Pro31, Glu32, Glu33, Asp34, Ile36, Thr37, Trp38, and Thr39.

Site 14, which is located near Site 9 but is distal to Site 8, comprises IL-12 p40 amino acid residues Gly48, Ser49, Gly50, Lys51, Thr52, Leu53, and Thr54.

Site 15, which is located near Site 9 but is distal to Site 8, comprises IL-12 p40 amino acid residues Gly64, Gln65, Thr67, Lys68, His69, Lys70, Gly71, Gly72, Glu73, Val74, Leu75, Ser76, and His77.

Site 16, which is located near Site 10 but is distal to Site 8, comprises IL-12 p40 amino acid residues Ile55, Gln56, Val57, Ly58, Glu59, Phe60, Asp62, Ala63, and Tyr66.

Site 17, which is located near Site 10 but is distal to Site 8, comprises IL-12 p40 amino acid residues Thr124, Thr125, Ile126, Ser127, Thr128, Asp129, Leu130, and Thr131.

Site 18, which is located near Site 10 but is distal to Site 8, comprises IL-12 p40 amino acid residues His194, Lys195, Leu196, and Lys197.

Thus, the present invention also provides a class of antibodies that bind to Site 9, but not Site 8, and which in addition bind to one or more sites selected from the group consisting of Site 13, Site 14, and Site 15. In addition, the present invention provides a class of antibodies that bind to Site 10, but not Site 8, and which in addition bind to one or more sites selected from the group consisting of Site 16, Site 17, and Site 18. Furthermore, because of the three-dimensional disposition of these Sites 9, 10, and 13-17, the present invention also provides antibodies that bind to Site 9, but not Site 8, and in addition bind to one or more sites selected from the group consisting of Site 13, Site 14, Site 15, Site 16, Site 17, and Site 18. The present invention further provides antibodies that bind to Site 10, but not Site 8, and in addition bind to one or more sites selected from the group consisting of Site 13, Site 14, Site 15, Site 16, Site 17, and Site 18.

### D. Engineered and Modified Antibodies

The V_{H} and/or V_{L} sequences of an antibody prepared according the methods of the present invention and may be used as starting material to engineer a modified antibody, which modified antibody may have altered properties from the starting antibody. An antibody can be engineered by modifying one or more residues within one or both of the original variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Framework sequences for antibodies can be obtained from public DNA databases or published references that include germline antibody gene sequences. For example, germline DNA sequences for human heavy and light chain variable region genes can be found in the “VBase” human germline sequence database (available on the Internet at mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference. As another example, the germline DNA sequences for human heavy and light chain variable region genes can be found in the Genbank database.

In one embodiment, the antibodies of the invention that bind the p40 subunit of IL-12/IL-23 comprise a heavy chain variable region derived from a member of the V_{H}3 family of germline sequences, and a light chain variable region derived from a member of the Vλ1 family of germline sequences. Moreover, the skilled artisan will appreciate that any member of the V_{H}3 family heavy chain sequence can be combined with any member of the Vλ1 family light chain sequence.

Antibody protein sequences are compared against a compiled protein sequence database using one of the sequence similarity searching methods called the Gapped BLAST (Altschul et al. (1997) Nucleic Acids Research 25:3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (vbase.mrc-cpe.cam.ac.uk/vbase1/list2.php) are translated and the region between and including FR1 through FR3 framework region is retained. The database sequences have an average length of 98 residues. Duplicate sequences which are exact matches over the entire length of the protein are removed. A BLAST search for proteins using the program blastp with default, standard parameters except the low complexity filter, which is turned off, and the substitution matrix of BLOSUM62, filters for the top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Identified V_{H} CDR1, CDR2, and CDR3 sequences, and the V_{L} CDR1, CDR2, and CDR3 sequences, can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays known in the art. For example, an antibody of the present invention may be mutated to create a library, which may then be screened for binding to a p40 subunit of IL-12/IL-23. Preferably conservative modifications (as discussed above) are introduced. The mutations may be amino acid substitutions, additions or deletions, but are preferably substitutions. Moreover, typically no more than one, two, three, four or five residues within a CDR region are altered.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. These strategies will be effective as long as the binding of the antibody to the p40 subunit of IL-12/IL-23 is not compromised.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in International PCT Application No. PCT/US08/73569 (PCT Publication No. WO 2009/026274), which is hereby incorporated by reference in its entirety. Such modifications include, but are not limited to, the replacement of an existing amino acid residue at or near the C-terminus of a full-length heavy chain sequence, as well as the introduction of a cysteine-containing extension to the c-terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C-terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase alpha-L-fucosidase removes fucosyl residues from antibodies (Tarentino, A. L. et al. (1975) Biochem. 14:5516-23).

Additionally or alternatively, an antibody can be made that has an altered type of glycosylation, wherein that alteration relates to the level of sialyation of the antibody. Such alterations are described in PCT Publication No. WO/2007/084926 to Dickey et al, and PCT Publication No. WO/2007/055916 to Ravetch et al., both of which are incorporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al. J. Virology, 15 (4), 882-893 (1975) and in Leibiger et al., Biochem J., 338, 529-538 (1999), respectively. Desialylated antibodies may be further purified by using affinity chromatography. Alternatively, one may employ methods to increase the level of sialyation, such as by employing sialytransferase enzymes. Conditions of such a reaction are generally described in Basset et al., Scandinavian Journal of Immunology, 51 (3), 307-311 (2000).

Another modification of the antibodies herein that is contemplated by the invention is pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono(C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al. As such, the methods of pegylation described here also apply the peptidic molecules of the invention described below.

### E. Antibody Fragments and Antibody Mimetics

The instant invention is not limited to traditional antibodies and may be practiced through the use of antibody fragments and antibody mimetics. As detailed below, a wide variety of antibody fragment and antibody mimetic technologies have now been developed and are widely known in the art. While a number of these technologies, such as domain antibodies, Nanobodies, and UniBodies make use of fragments of, or other modifications to, traditional antibody structures, there are also alternative technologies, such as Adnectins, Affibodies, DARPins, Anticalins, Avimers, Versabodies, Aptamers and SMIPS that employ binding structures that, while they mimic traditional antibody binding, are generated from and function via distinct mechanisms. Some of these alternative structures are reviewed in Gill and Damle (2006) 17: 653-658.

Domain Antibodies (dAbs) are the smallest functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, domain antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The Nanoclone method (see, e.g., WO 06/079372, which is herein incorporated by reference in its entirety) is a proprietary method for generating Nanobodies against a desired target, based on automated high-throughout selection of B-cells and could be used in the context of the instant invention.

UniBodies are another antibody fragment technology, however this technology is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, UniBodies may function to inhibit or silence, but not kill, the cells to which they are bound. Additionally, UniBody binding to cancer cells do not stimulate them to proliferate. Furthermore, because UniBodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

Adnectin molecules are engineered binding proteins derived from one or more domains of the fibronectin protein. Fibronectin exists naturally in the human body. It is present in the extracellular matrix as an insoluble glycoprotein dimer and also serves as a linker protein. It is also present in soluable form in blood plasma as a disulphide linked dimer. The plasma form of fibronectin is synthesized by liver cells (hepatocytes), and the ECM form is made by chondrocytes, macrophages, endothelial cells, fibroblasts, and some cells of the epithelium (see Ward M., and Marcey, D., callutheran.edu/Academic_Programs/Departments/BioDev/omm/fibro/fibro.htm). As mentioned previously, fibronectin may function naturally as a cell adhesion molecule, or it may mediate the interaction of cells by making contacts in the extracellular matrix. Typically, fibronectin is made of three different protein modules, type I, type II, and type III modules. For a review of the structure of function of the fibronectin, see Pankov and Yamada (2002) J Cell Sci.; 115 (Pt 20):3861-3, Hohenester and Engel (2002) 21:115-128, and Lucena et al. (2007) Invest Clin. 48:249-262.

In a preferred embodiment, adnectin molecules are derived from the fibronectin type III domain by altering the native protein which is composed of multiple beta strands distributed between two beta sheets. Depending on the originating tissue, fibronecting may contain multiple type III domains which may be denoted, e.g., ¹Fn3, ²Fn3, ³Fn3, etc. The ¹⁰Fn3 domain contains an integrin binding motif and further contains three loops which connect the beta strands. These loops may be thought of as corresponding to the antigen binding loops of the IgG heavy chain, and they may be altered by methods discussed below to specifically bind a target of interest, e.g., the p40 subunit of IL-12/IL-23. Preferably, a fibronectin type III domain useful for the purposes of this invention is a sequence which exhibits a sequence identity of at least 30%, at least 40%, at least 50%, at least 60%, at least 70%, at least 80%, at least 90%, or at least 95% to the sequence encoding the structure of the fibronectin type III molecule which can be accessed from the Protein Data Bank (PDB, rcsb.org/pdb/home/home.do) with the accession code: 1ttg. Adnectin molecules may also be derived from polymers of ¹⁰Fn3 related molecules rather than a simple monomeric ¹⁰Fn3 structure.

Although the native ¹⁰Fn3 domain typically binds to integrin, ¹⁰Fn3 proteins adapted to become adnectin molecules are altered so to bind antigens of interest, e.g., the p40 subunit of IL-12/IL-23. In one embodiment, the alteration to the ¹⁰Fn3 molecule comprises at least one mutation to a beta strand. In a preferred embodiment, the loop regions which connect the beta strands of the ¹⁰Fn3 molecule are altered to bind to the p40 subunit of IL-12/IL-23.

The alterations in the ¹⁰Fn3 may be made by any method known in the art including, but not limited to, error prone PCR, site-directed mutagenesis, DNA shuffling, or other types of recombinational mutagenesis which have been referenced herein. In one example, variants of the DNA encoding the ¹⁰Fn3 sequence may be directly synthesized in vitro, and later transcribed and translated in vitro or in vivo. Alternatively, a natural ¹⁰Fn3 sequence may be isolated or cloned from the genome using standard methods (as performed, e.g., in U.S. Pat. Application No. 20070082365), and then mutated using mutagenesis methods known in the art.

In one embodiment, a target protein, e.g., the p40 subunit of IL-12/IL-23, may be immobilized on a solid support, such as a column resin or a well in a microtiter plate. The target is then contacted with a library of potential binding proteins. The library may comprise ¹⁰Fn3 clones or adnectin molecules derived from the wild type ¹⁰Fn3 by mutagenesis/randomization of the ¹⁰Fn3 sequence or by mutagenesis/randomization of the ¹⁰Fn3 loop regions (not the beta strands). In a preferred embodiment the library may be an RNA-protein fusion library generated by the techniques described in Szostak et al., U.S. Ser. No. 09/007,005 and 09/247,190; Szostak et al., WO989/31700; and Roberts & Szostak (1997) 94:12297-12302. The library may also be a DNA-protein library (e.g., as described in Lohse, U.S. Ser. No. 60/110,549, U.S. Ser. No. 09/459,190, and WO 00/32823). The fusion library is then incubated with the immobilized target (e.g., the p40 subunit of IL-12/IL-23) and the solid support is washed to remove non-specific binding moieties. Tight binders are then eluted under stringent conditions and PCR is used to amply the genetic information or to create a new library of binding molecules to repeat the process (with or without additional mutagenesis). The selection/mutagenesis process may be repeated until binders with sufficient affinity to the target are obtained. Adnectin molecules for use in the present invention may be engineered using the PROfusion™ technology employed by Adnexus, a Briston-Myers Squibb company. The PROfusion technology was created based on the techniques referenced above (e.g., Roberts & Szostak (1997) 94:12297-12302). Methods of generating libraries of altered ¹⁰Fn3 domains and selecting appropriate binders which may be used with the present invention are described fully in the following U.S. patent and patent application documents and are incorporated herein by reference: U.S. Pat. Nos. 7,115,396; 6,818,418; 6,537,749; 6,660,473; 7,195,880; 6,416,950; 6,214,553; 6623926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. Pat. Application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. The generation of diversity in fibronectin type III domains, such as ¹⁰Fn3, followed by a selection step may be accomplished using other methods known in the art such as phage display, ribosome display, or yeast surface display, e.g., Lipov\check{s}ek et al. (2007) Journal of Molecular Biology 368: 1024-1041; Sergeeva et al. (2006) Adv Drug Deliv Rev. 58:1622-1654; Petty et al. (2007) Trends Biotechnol. 25: 7-15; Rothe et al. (2006) Expert Opin Biol Ther. 6:177-187; and Hoogenboom (2005) Nat Biotechnol. 23:1105-1116.

It should be appreciated by one of skill in the art that the methods references cited above may be used to derive antibody mimics from proteins other than the preferred ¹⁰Fn3 domain. Additional molecules which can be used to generate antibody mimics via the above referenced methods include, without limitation, human fibronectin modules ¹Fn3-⁹Fn3 and ¹¹Fn3-¹⁷Fn3 as well as related Fn3 modules from non-human animals and prokaryotes. In addition, Fn3 modules from other proteins with sequence homology to ¹⁰Fn3, such as tenascins and undulins, may also be used. Other exemplary proteins having immunoglobulin-like folds (but with sequences that are unrelated to the V_{H} domain) include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule P0, CD8, CD4, CD2, class I MHC, T-cell antigen receptor, CD1, C2 and I-set domains of VCAM-1, I-set immunoglobulin fold of myosin-binding protein C, I-set immunoglobulin fold of myosin-binding protein H, I-set immunoglobulin-fold of telokin, telikin, NCAM, twitchin, neuroglian, growth hormone receptor, erythropoietin receptor, prolactin receptor, GC-SF receptor, interferon-gamma receptor, beta-galactosidase/glucuronidase, beta-glucuronidase, and transglutaminase. Alternatively, any other protein that includes one or more immunoglobulin-like folds may be utilized to create a adnecting like binding moiety. Such proteins may be identified, for example, using the program SCOP (Murzin et al., J. Mol. Biol. 247:536 (1995); Lo Conte et al., Nucleic Acids Res. 25:257 (2000).

An aptamer is another type of antibody-mimetic which is encompassed by the present invention. Aptamers are typically small nucleotide polymers that bind to specific molecular targets. Aptamers may be single or double stranded nucleic acid molecules (DNA or RNA), although DNA based aptamers are most commonly double stranded. There is no defined length for an aptamer nucleic acid; however, aptamer molecules are most commonly between 15 and 40 nucleotides long.

Aptamers often form complex three-dimensional structures which determine their affinity for target molecules. Aptamers can offer many advantages over simple antibodies, primarily because they can be engineered and amplified almost entirely in vitro. Furthermore, aptamers often induce little or no immune response.

Aptamers may be generated using a variety of techniques, but were originally developed using in vitro selection (Ellington and Szostak. (1990) Nature. 346 (6287):818-22) and the SELEX method (systematic evolution of ligands by exponential enrichment) (Schneider et al. 1992. J Mol Biol. 228 (3):862-9) the contents of which are incorporated herein by reference. Other methods to make and uses of aptamers have been published including Klussmann. The Aptamer Handbook Functional Oligonucleotides and Their Applications. ISBN: 978-3-527-31059-3; Ulrich et al. 2006. Comb Chem High Throughput Screen 9 (8):619-32; Cerchia and de Franciscis. 2007. Methods Mol Biol. 361:187-200; Ireson and Kelland. 2006. Mol Cancer Ther. 2006 5 (12):2957-62; U.S. Pat. Nos. 5,582,981; 5,840,867; 5,756,291; 6,261,783; 6,458,559; 5,792,613; 6,111,095; and U.S. patent application Ser. Nos. 11/482,671; 11/102,428; 11/291,610; and 10/627,543 which are all incorporated herein by reference.

The SELEX method is clearly the most popular and is conducted in three fundamental steps. First, a library of candidate nucleic acid molecules is selected from for binding to specific molecular target. Second, nucleic acids with sufficient affinity for the target are separated from non-binders. Third, the bound nucleic acids are amplified, a second library is formed, and the process is repeated. At each repetition, aptamers are chosen which have higher and higher affinity for the target molecule. SELEX methods are described more fully in the following publications, which are incorporated herein by reference: Bugaut et al. 2006. 4 (22):4082-8; Stoltenburg et al. 2007 Biomol Eng. 2007 24 (4):381-403; and Gopinath. 2007. Anal Bioanal Chem. 2007. 387 (1):171-82.

An “aptamer” of the invention also been includes aptamer molecules made from peptides instead of nucleotides. Peptide aptamers share many properties with nucleotide aptamers (e.g., small size and ability to bind target molecules with high affinity) and they may be generated by selection methods that have similar principles to those used to generate nucleotide aptamers, for example Baines and Colas. 2006. Drug Discov Today. 11 (7-8):334-41; and Bickle et al. 2006. Nat Protoc. 1 (3):1066-91 which are incorporated herein by reference.

Affibody molecules represent a new class of affinity proteins based on a 58-amino acid residue protein domain, derived from one of the IgG-binding domains of staphylococcal protein A. This three helix bundle domain has been used as a scaffold for the construction of combinatorial phagemid libraries, from which Affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Ronmark J, Harmon M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

Lipocalins are cloned and their loops are subjected to engineering in order to create Anticalins. Libraries of structurally diverse Anticalins have been generated and Anticalin display allows the selection and screening of binding function, followed by the expression and production of soluble protein for further analysis in prokaryotic or eukaryotic systems. Studies have successfully demonstrated that Anticalins can be developed that are specific for virtually any human target protein can be isolated and binding affinities in the nanomolar or higher range can be obtained.

Anticalins can also be formatted as dual targeting proteins, so-called Duocalins. A Duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as Duocalins have significant potential in targeting cell surface molecules in disease, mediating agonistic effects on signal transduction pathways or inducing enhanced internalization effects via binding and clustering of cell surface receptors. Furthermore, the high intrinsic stability of Duocalins is comparable to monomeric Anticalins, offering flexible formulation and delivery potential for Duocalins.

Additional information regarding Anticalins can be found in U.S. Pat. No. 7,250,297 and International Patent Application Publication No. WO 99/16873, both of which are hereby incorporated by reference in their entirety.

Another antibody mimetic technology useful in the context of the instant invention are Avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding Avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for Versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding Versabodies can be found in U.S. Patent Application Publication No. 2007/0191272 which is hereby incorporated by reference in its entirety.

SMIPs™ (Small Modular ImmunoPharmaceuticals-Trubion Pharmaceuticals) are engineered to maintain and optimize target binding, effector functions, in vivo half life, and expression levels. SMIPS consist of three distinct modular domains. First they contain a binding domain which may consist of any protein which confers specificity (e.g., cell surface receptors, single chain antibodies, soluble proteins, etc). Secondly, they contain a hinge domain which serves as a flexible linker between the binding domain and the effector domain, and also helps control multimerization of the SMIP drug. Finally, SMIPS contain an effector domain which may be derived from a variety of molecules including Fc domains or other specially designed proteins. The modularity of the design, which allows the simple construction of SMIPs with a variety of different binding, hinge, and effector domains, provides for rapid and customizable drug design.

More information on SMIPs, including examples of how to design them, may be found in Zhao et al. (2007) Blood 110:2569-77 and the following U.S. Pat. App. Nos. 20050238646; 20050202534; 20050202028; 20050202023; 20050202012; 20050186216; 20050180970; and 20050175614.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25 (8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

### F. Antibody Physical Properties

The antibodies of the present invention, which bind to the p40 subunit of IL-12/IL-23, may be further characterized by the various physical properties. Various assays may be used to detect and/or differentiate different classes of antibodies based on these physical properties.

In some embodiments, antibodies of the present invention may contain one or more glycosylation sites in either the light or heavy chain variable region. The presence of one or more glycosylation sites in the variable region may result in increased immunogenicity of the antibody or an alteration of the pK of the antibody due to altered antigen binding (Marshall et al (1972) Annu Rev Biochem 41:673-702; Gala F A and Morrison S L (2004) J Immunol 172:5489-94; Wallick et al (1988) J Exp Med 168:1099-109; Spiro R G (2002) Glycobiology 12:43R-56R; Parekh et al (1985) Nature 316:452-7; Mimura et al. (2000) Mol Immunol 37:697-706). Glycosylation has been known to occur at motifs containing an N-X-S/T sequence. Variable region glycosylation may be tested using a Glycoblot assay, which cleaves the antibody to produce a Fab, and then tests for glycosylation using an assay that measures periodate oxidation and Schiff base formation. Alternatively, variable region glycosylation may be tested using Dionex light chromatography (Dionex-LC), which cleaves saccharides from a Fab into monosaccharides and analyzes the individual saccharide content. In some instances, it may be preferred to have an antibody that does not contain variable region glycosylation. This can be achieved either by selecting antibodies that do not contain the glycosylation motif in the variable region or by mutating residues within the glycosylation motif using standard techniques well known in the art.

Each antibody will have a unique isoelectric point (pI), but generally antibodies will fall in the pH range of between 6 and 9.5. The pI for an IgG1 antibody typically falls within the pH range of 7-9.5 and the pI for an IgG4 antibody typically falls within the pH range of 6-8. Antibodies may have a pI that is outside this range. Although the effects are generally unknown, there is speculation that antibodies with a pI outside the normal range may have some unfolding and instability under in vivo conditions. The isoelectric point may be tested using a capillary isoelectric focusing assay, which creates a pH gradient and may utilize laser focusing for increased accuracy (Janini et al (2002) Electrophoresis 23:1605-11; Ma et al. (2001) Chromatographia 53:S75-89; Hunt et al (1998) J Chromatogr A 800:355-67). In some instances, it is preferred to have an antibody that contains a pI value that falls in the normal range. This can be achieved either by selecting antibodies with a pI in the normal range, or by mutating charged surface residues using standard techniques well known in the art.

Each antibody will have a melting temperature that is indicative of thermal stability (Krishnamurthy R and Manning M C (2002) Curr Pharm Biotechnol 3:361-71). A higher thermal stability indicates greater overall antibody stability in vivo. The melting point of an antibody may be measure using techniques such as differential scanning calorimetry (Chen et al (2003) Pharm Res 20:1952-60; Ghirlando et al (1999) Immunol Lett 68:47-52). T_{M1} indicates the temperature of the initial unfolding of the antibody. T_{M2} indicates the temperature of complete unfolding of the antibody. Generally, it is preferred that the T_{M1} of an antibody of the present invention is greater than 60° C., preferably greater than 65° C., even more preferably greater than 70° C. Alternatively, the thermal stability of an antibody may be measure using circular dichroism (Murray et al. (2002) J. Chromatogr Sci 40:343-9).

In a preferred embodiment, antibodies that do not rapidly degrade may be desired. Fragmentation of an antibody may be measured using capillary electrophoresis (CE) and MALDI-MS, as is well understood in the art (Alexander A J and Hughes D E (1995) Anal Chem 67:3626-32).

In another preferred embodiment, antibodies are selected that have minimal aggregation effects. Aggregation may lead to triggering of an unwanted immune response and/or altered or unfavorable pharmacokinetic properties. Generally, antibodies are acceptable with aggregation of 25% or less, preferably 20% or less, even more preferably 15% or less, even more preferably 10% or less and even more preferably 5% or less. Aggregation may be measured by several techniques well known in the art, including size-exclusion column (SEC) high performance liquid chromatography (HPLC), and light scattering to identify monomers, dimers, trimers or multimers.

### V. Production of Antibodies of the Invention

### A. Production of Polyclonal Antibodies of the Invention

Polyclonal antibodies of the present invention can be produced by a variety of techniques that are well known in the art. Polyclonal antibodies are derived from different B-cell lines and thus may recognize multiple epitopes on the same antigen. Polyclonal antibodies are typically produced by immunization of a suitable mammal with the antigen of interest, e.g., the p40 subunit of IL-12/IL-23. Animals often used for production of polyclonal antibodies are chickens, goats, guinea pigs, hamsters, horses, mice, rats, sheep, and, most commonly, rabbit. Standard methods to produce polyclonal antibodies are widely known in the art and can be combined with the methods of the present invention (e.g., research.cm.utexas.edu/bkitto/Kittolabpage/Protocols/Immunology/PAb.html; U.S. Pat. Nos. 4,719,290, 6,335,163, 5,789,208, 2,520,076, 2,543,215, and 3,597,409, the entire contents of which are incorporated herein by reference.

### B. Production of Monoclonal Antibodies of the Invention

Monoclonal antibodies (mAbs) of the present invention can be produced by a variety of techniques, including conventional monoclonal antibody methodology e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes. It should be noted that antibodies (monoclonal or polyclonal) or antigen binding portions thereof, may be raised to any epitope on the p40 subunit of IL-12/IL-23, including any conformational, discontinuous, or linear epitopes described herein.

Several methods known in the art are useful for specifically selecting an antibody or antigen binding fragment thereof that specifically binds a discontinuous epitope of interest. For example, the techniques disclosed in U.S. Publication No. 2005/0169925, the entire contents of which are incorporated herein by reference, allow for the selection of an antibody which binds to two different peptides within a protein sequence. Such methods may be used in accordance with the present invention to specifically target the conformational and discontinuous epitopes disclosed herein. If the conformational epitope is a protein secondary structure, such structures often form easily in smaller peptides (e.g., <50 amino acids). Thus, immunizing an animal with smaller peptides could capture some conformational epitopes. Alternatively, two small peptides which comprise a conformational epitope (e.g., the peptides identified in Table 5) may be connected via a flexible linker (e.g., polyglycol, or a stretch of polar, uncharged amino acids). The linker will allow the peptides to explore various interaction orientations. Immunizing with this construct, followed by appropriate screening could allow for identification of antibodies directed to a conformational epitope. In a preferred embodiment, peptides to specific conformational or linear epitopes may be generated by immunizing an animal with a particular domain of the p40 subunit of IL-12/IL-23 (e.g., the epitopes described in sections II(A) and II(C), including the Sites described in Table 3 and the Epitopes described in Table 4 above) and subsequently screening for antibodies which bind the epitope of interest. In one embodiment cryoelectron microscopy (Jiang et al. (2008) Nature 451, 1130-1134; Joachim (2006) Oxford University Press ISBN:0195182189) may be used to identify the epitopes bound by an antibody or antigen binding fragment of the invention. In another embodiment, the p40 subunit of IL-12/IL-23 or a domain thereof may be crystallized with the bound antibody or antigen binding fragment thereof and analyzed by X-ray crystallography to determine the precise epitopes that are bound. In addition, epitopes may be mapped by replacing portions of the p40 subunit of IL-12/IL-23 sequence with the corresponding sequences from mouse or another species. Antibodies directed to epitopes of interest will selectively bind the human sequence regions and, thus, it is possible to sequentially map target epitopes. This technique of chimera based epitope mapping has been used successfully to identify epitopes in various settings (see Henriksson and Pettersson (1997) Journal of Autoimmunity. 10 (6):559-568; Netzer et al. (1999) J Biol. Chem. 1999 Apr. 16; 274 (16):11267-74; Hsia et al. (1996) Mol. Microbiol. 19, 53-63, the entire contents of which are incorporated herein by reference).

If a p40 subunit of IL-12/IL-23 domain of interest is glycosylated, antibodies or antigen binding portions thereof (and other antibody mimetics of the invention), may be raised such that they bind to the relevant amino acid and/or sugar residues. The p40 subunit of human IL-12/23 contains 10 cysteine residues and four potential N-linked glycosylation sites. The glycosylation pattern of the p40 subunit of IL-12/23 is further described at least in: Yoon et al. 2000 EMBO 19 (14):3530-3541; Gubler et al. 1991 Proc. Natl. Acad. Sci. USA 88:4143-4147; and Brunda et al. 1994 J. Leukocyte Biol. 55:280-288, the entire contents of each of which are hereby incorporated by reference herein. Thus, it is contemplated that antibodies or antigen binding portions thereof (and other moieties of the invention), may be raised such that they also bind to sugar residues which may be attached to any epitope identified herein. For this purpose, an antigenic peptide of interest may, be produced in an animal cell such that it gets properly glycosylated and the glycosylated antigenic peptide may then be used to immunize an animal. Suitable cells and techniques for producing glycosylated peptides are known in the art and described further below (see, for example, the technologies available from GlycoFi, Inc., Lebanon, N.H. and BioWa; Princeton, N.J.). The proper glycosylation of a peptide may be tested using any standard methods such as isoelectric focusing (IEF), acid hydrolysis (to determine monosaccharide composition), chemical or enzymatic cleavage, and mass spectrometry (MS) to identify glycans. The technology offered by Procognia (procognia.com) which uses a lectin-based array to speed up glycan analysis may also be used. O-glycosylation specifically may be detected using techniques such as reductive alkaline cleavage or “beta elimination”, peptide mapping, liquid chromatography, and mass spectrometry or any combination of these techniques.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.). Alternatively, a humanized antibody may be designed at the DNA or protein level, given knowledge of human and non-human sequences. Such antibodies may be directly synthesized chemically, or the DNA may be synthesized and expressed in vitro or in vivo to produce a humanized antibody.

In a preferred embodiment, the antibodies of the invention are human monoclonal antibodies. Such human monoclonal antibodies directed against a domain or epitope of the p40 subunit of IL-12/IL-23 as described herein, can be generated using transgenic or transchromosomic mice carrying parts of the human immune system rather than the mouse system. These transgenic and transchromosomic mice include mice referred to herein as HuMAb mice and KM Mice™, respectively, and are collectively referred to herein as “human Ig mice.”

The HuMAb Mouse® (Medarex, Inc.) contains human immunoglobulin gene miniloci that encode unrearranged human heavy (μ and γ) and κ light chain immunoglobulin sequences, together with targeted mutations that inactivate the endogenous μ and κ chain loci (see e.g., Lonberg, et al. (1994) Nature 368 (6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication WO 02/43478 to Ishida et al.

Still further, alternative transgenic animal systems expressing human immunoglobulin genes are available in the art and can be used to raise the antibodies of the invention. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise the antibodies of the invention. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise the antibodies of the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al. In one embodiment, human monoclonal antibodies of the invention can be prepared using phage display techniques as described in U.S. Pat. No. 6,914,128, the entire contents of which are incorporated by reference herein. In another embodiment, human monoclonal antibodies of the invention can be prepared from human antibody libraries such as those described in U.S. Pat. No. 6,914,128, the entire contents of which are incorporated by reference herein.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In another embodiment, antibodies of the invention may be raised using well known phage display techniques, as described in Marks, J. D., et al. ((1991). J. Mol. Biol. 222, 581), Nissim, A., et al. ((1994). EMBO J. 13, 692) and U.S. Pat. Nos. 6,794,132; 6,562,341; 6,057,098; 5,821,047; and 6,512,097.

In a further embodiment, antibodies of the present invention may be found using yeast cell surface display technology as described, for example, in U.S. Pat. Nos. 6,423,538; 6,300,065; 6,696,251; 6,699,658.

Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG Alternatively, the single cell suspension of splenic lymphocytes from immunized mice can be fused using an electric field based electrofusion method, using a CytoPulse large chamber cell fusion electroporator (CytoPulse Sciences, Inc., Glen Burnie Md.). Cells are plated at approximately 2×10⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

### Generation of Transfectomas Producing Monoclonal Antibodies of the Invention

Antibodies of the invention also can be produced in a host cell transfectoma (a type of hybridoma) using, for example, a combination of recombinant DNA techniques and gene transfection methods as is well known in the art (e.g., Morrison, S. (1985) Science 229:1202).

For example, to express the antibodies, or antibody fragments thereof, isolated nucleic acid molecules, e.g., DNA, encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences.

The phrase “nucleic acid molecule” includes DNA molecules and RNA molecules. A nucleic acid molecule may be single-stranded or double-stranded, but preferably is double-stranded DNA.

The phrase “isolated nucleic acid molecule”, as used herein in reference to nucleic acids encoding antibodies or antibody portions (e.g., VH, VL, CDR3) that bind hIL-12 including “isolated antibodies”), includes a nucleic acid molecule in which the nucleotide sequences encoding the antibody or antibody portion are free of other nucleotide sequences encoding antibodies or antibody portions that bind antigens other than hIL-12, which other sequences may naturally flank the nucleic acid in human genomic DNA. Thus, for example, an isolated nucleic acid of the invention encoding a VH region of an anti-IL-12 antibody contains no other sequences encoding other VH regions that bind antigens other than IL-12. The phrase “isolated nucleic acid molecule” is also intended to include sequences encoding bivalent, bispecific antibodies, such as diabodies in which VH and VL regions contain no other sequences other than the sequences of the diabody.

The term “vector” includes a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the described antibodies can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{K} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. In certain embodiments, the host cell may be a eukaryotic cell or a prokaryotic cell.

The term “regulatory sequence” is intended to include promoters, enhancers and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr− host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

In view of the foregoing, another aspect of the invention pertains to nucleic acid, vector and host cell compositions that can be used for recombinant expression of the antibodies and antibody portions of the invention. In one embodiment, the invention features isolated nucleic acids that encode CDRs of J695, and/or the full heavy and/or light chain variable region of J695. Accordingly, in one embodiment, the invention provides an isolated nucleic acid encoding an antibody heavy chain variable region that encodes the J695 heavy chain CDR3 as set forth in the amino acid sequence of SEQ ID NO:1. In one embodiment, the nucleic acid encoding the antibody heavy chain variable region further encodes a J695 heavy chain CDR2 as set forth in the amino acid sequence of SEQ ID NO: 1. In another embodiment, the nucleic acid encoding the antibody heavy chain variable region further encodes a J695 heavy chain CDR1 as set forth in the amino acid sequence of SEQ ID NO: 1. In another embodiment, the isolated nucleic acid encodes an antibody heavy chain variable region comprising the amino acid sequence of SEQ ID NO: 1 (the full VH region of J695). In various embodiments, the nucleic acids encode an antibody heavy chain variable region further comprising one or more substitutions as described herein, e.g., as described in sections II(A)(2) and II(B) above.

In other embodiments, the invention provides an isolated nucleic acid encoding an antibody light chain variable region that encodes the J695 light chain CDR3 as set forth in the amino acid sequence of SEQ ID NO: 2. In one embodiment, the nucleic acid encoding the antibody light chain variable region further encodes a J695 light chain CDR2 as set forth in the amino acid sequence of SEQ ID NO: 2. In one embodiment, the nucleic acid encoding the antibody light chain variable region further encodes a J695 light chain CDR1 as set forth in the amino acid sequence of SEQ ID NO: 2. In another embodiment, the isolated nucleic acid encodes an antibody light chain variable region comprising the amino acid sequence of SEQ ID NO: 2 (the full VL region of J695). In various embodiments, the nucleic acids encode an antibody light chain variable region further comprising one or more substitutions as described herein, e.g., as described in sections II(A)(2) and II(B) above.

The invention also provides recombinant expression vectors encoding both an antibody heavy chain and an antibody light chain. For example, in one embodiment, the invention provides a recombinant expression vector encoding: a) an antibody heavy chain having a variable region comprising the amino acid sequence of SEQ ID NO: 1; and b) an antibody light chain having a variable region comprising the amino acid sequence of SEQ ID NO: 2, and further comprising one or more substitutions as described herein, e.g., as described in sections II(A)(2) and II(B) above.

The invention also provides host cells into which one or more of the recombinant expression vectors of the invention have been introduced. Still further the invention provides a method of synthesizing a recombinant human antibody of the invention by culturing a host cell of the invention in a suitable culture medium until a recombinant human antibody of the invention is synthesized. The method can further comprise isolating the recombinant human antibody from the culture medium.

Preferred mammalian host cells for expressing the recombinant antibodies of the invention include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

C. Characterization of Antibody Binding to the p40 Subunit of IL-12 and/or IL-23

The present invention provides anti-p40 subunit of IL-12 and/or anti-IL-23 antibodies (also referred to herein as IL-12p40 antibodies and IL-23p40 antibodies, respectively) that specifically bind to the p40 subunit of IL-12 and/or IL-23. As used herein, an antibody that “specifically binds” to a p40 subunit of IL-12 and/or IL-23 is intended to refer to an antibody that binds to a p40 subunit of IL-12 and/or IL-23 with a K_{d} of 1×10⁻⁷ M or less, more preferably 5×10⁻⁸ M or less, more preferably 1×10⁻⁸ M or less, more preferably 5×10⁻⁹ M or less, more preferably 1×10⁻⁹ M or less, more preferably 5×10⁻¹⁰ M or less, and more preferably 1×10⁻¹⁰ M or less, and more preferably 1×10⁻¹¹ or less.

The term “does not substantially bind” to a protein or cells, as used herein, means does not bind or does not bind with a high affinity to the protein or cells, i.e. binds to the protein or cells with a K_{d} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

Anti-p40 subunit of IL-12 and/or anti-IL-23 antibodies provided by the present invention can optionally be characterized by high affinity binding to the p40 subunit of IL-12 and/or IL-23. The affinity or avidity of an antibody for an antigen can be determined experimentally using any suitable method. (See, for example, Berzofsky, et al, “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., Kₐ) are preferably made with standardized solutions of antibody and antigen, and a standardized buffer, such as the buffer described herein. Standard assays to evaluate the binding ability of the antibodies toward the p40 subunit of IL-12/IL-23 are known in the art, including for example, ELISAs, Western blots and RIAs. The binding kinetics (e.g., binding affinity) of the antibodies also can be assessed by standard assays known in the art, such as by ELISA, Scatchard and Biacore analysis.

The term “K_{d},” as used herein, is intended to refer to the dissociation constant, of a particular antibody-antigen interaction and is expressed as a molar concentration (M). K_{d} values for antibodies can be determined using methods well established in the art. A preferred method for determining the K_{d} of an antibody is by using surface plasmon resonance, preferably using a biosensor system such as a Biacore® system.

The dissociation rate constant (k_{off}) of an antibody can be determined by surface plasmon resonance. Generally, surface plasmon resonance analysis measures real-time binding interactions between ligand (e.g., recombinant human IL-12 immobilized on a biosensor matrix) and analyte (antibodies in solution) by surface plasmon resonance (SPR) using the BIAcore system (Pharmacia Biosensor, Piscataway, N.J.). Surface plasmon analysis can also be performed by immobilizing the analyte (antibodies on a biosensor matrix) and presenting the ligand (e.g., recombinant IL-12 in solution).

The phrase “surface plasmon resonance” includes an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

In certain embodiments, the antibodies provided by the invention can bind to the p40 subunit of IL-12 (e.g., human IL-12) and/or IL-23 (e.g., human IL-23) with a wide range of affinities (K_{d}). In one embodiment, an antibody of the present invention binds the p40 subunit of human IL-12 and/or IL-23 with high affinity. For example, an antibody can bind the p40 subunit of human IL-12 and/or human IL-23 with a K_{d} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein. In one embodiment, antibodies of the invention bind the p40 subunit of IL-12 and/or IL-23 with a K_{d} equal to or less than about 1×10⁻⁶ M. In one embodiment, antibodies of the invention bind the p40 subunit of IL-12 and/or IL-23 with a K_{d} equal to or less than about 1×10⁻⁷ M. In one embodiment, antibodies of the invention bind the p40 subunit of IL-12 and/or IL-23 with a K_{d} equal to or less than about 1×10⁻⁸ M. In one embodiment, antibodies of the invention bind the p40 subunit of IL-12 and/or IL-23 with a K_{d} equal to or less than about 1×10⁻⁹ M. In one embodiment, antibodies of the invention bind the p40 subunit of IL-12 and/or IL-23 with a K_{d} equal to or less than about 1×10⁻¹⁰ M. In one embodiment, antibodies of the invention bind the p40 subunit of IL-12 and/or IL-23 with a K_{d} equal to or less than about 1×10⁻¹¹ M. In one embodiment, antibodies of the invention bind the p40 subunit of IL-12 and/or IL-23 with a K_{d} equal to or less than about 1×10⁻¹² M. In one embodiment, antibodies of the invention bind the p40 subunit of IL-12 and/or IL-23 with a K_{d} equal to or less than about 1×10⁻¹³ M. In various embodiments, an antibody of the invention binds to a p40 subunit containing cytokine, e.g., IL-12 and/or IL-23, with a K_{d} of 5×10⁻⁸ M or less, a K_{d} of 1×10⁻⁸ M or less, a K_{d} of 5×10⁻⁹ M or less, a IQ of 1×10⁻⁹M or less, a K_{d} of 5×10⁻¹⁰ M or less, or a K_{d} of 1×10⁻¹⁰ M or less.

In certain other embodiments, the antibodies provided by the invention can bind to the p40 subunit of IL-12 (e.g., human IL-12) and/or IL-23 (e.g., human IL-23) with a k_{off} rate constant of 0.1 s⁻¹ or less, as determined by surface plasmon resonance. In one embodiment, the isolated IL-12, IL-23, and/or p40 subunit of IL-12 and/or IL-23 antibody, or an antigen-binding portion thereof, dissociates from IL-12, IL-23 and/or p40 subunit of IL-12 and/or IL-23 with a k_{off} rate constant of 1×10⁻² s⁻¹ or less. In more preferred embodiments, the isolated IL-12, IL-23 and/or the p40 subunit of IL-12 and/or IL-23 antibody, or an antigen-binding portion thereof, dissociates from IL-12, and/or human IL-23, and/or the p40 subunit of the same, with a k_{off} rate constant of 1×10⁻³ s⁻¹ or less. In more preferred embodiments, the isolated IL-12, IL-23 and/or p40 subunit of IL-12 and/or Il-23 antibody, or an antigen-binding portion thereof, dissociates from IL-12, and/or IL-23, and/or the p40 subunit of the same, with a k_{off} rate constant of 1×10⁴ s⁻¹ or less. In more preferred embodiments, the isolated IL-12, IL-23 and/or p40 subunit of IL-12 and/or Il-23 antibody, or an antigen-binding portion thereof, dissociates from IL-12, and/or IL-23, and/or the p40 subunit of the same, with a k_{off} rate constant of 1×10⁻⁵ s⁻¹ or less.

In various embodiments, the antibodies of the invention, or antigen-binding portions thereof, are neutralizing. Neutralization activity of antibodies provided by the present invention, or antigen binding portions thereof, can be assessed using one or more of several suitable in vitro assays described herein. A “neutralizing antibody” (or an “antibody that neutralizes the activity of the p40 subunit of IL-12 and/or IL-23” or an “antibody that neutralizes IL-12 and/or IL-23 activity”) includes an antibody whose binding to the p40 subunit of IL-12 and/or IL-23 results in inhibition of the biological activity of the p40 subunit of IL-12 and/or IL-23, e.g., the biological activity of IL-12 and/or IL-23. This inhibition of biological activity can be assessed by measuring one or more indicators of p40 subunit of IL-12/23 and/or IL-12 and/or IL-23 biological activity, such as inhibition of human phytohemagglutinin blast proliferation in a phytohemagglutinin blast proliferation assay (PHA assay), inhibition of IL-12-induced interferon gamma production by human blast cells (IFN gamma assay), or inhibition of receptor binding in an IL-12 (or IL-23) receptor binding assay (RBA assay), e.g., as described in detail in U.S. Pat. No. 6,914,128, the entire contents of which are incorporated by reference herein. These indicators of p40 subunit of IL-12/23 and/or IL-12 and/or IL-23 biological activity can be assessed by one or more of several standard in vitro or in vivo assays known in the art.

Anti-p40 subunit of IL-12/IL-23 antibodies can be evaluated for their ability to inhibit PHA blast proliferation (which proliferation is stimulated by IL-12). In a standard assay, serial dilutions of anti-p40 subunit of IL-12/IL-23 antibody are preincubated for 1 hour at 37° C., 5% CO₂ with 230 pg/ml hIL-12 in 100 ml RPMI complete medium in a microtiter plate (U-bottom, 96-well, Costar, Cambridge, Mass.). PHA blast cells are isolated, washed once and resuspended in RPMI complete medium to a cell density of 3×10⁵ cells/ml. PHA blasts (100 ml, 3×10⁴ cells) are added to the antibody/hIL-12 mixture, incubated for 3 days at 37° C., 5% CO₂ and labeled for 4-6 hours with 0.5 mCi/well (3H)-Thymidine (Amersham, Arlington Heights, Ill.). The culture contents are harvested onto glass fiber filters by means of a cell harvester (Tomtec, Orange, Conn.) and (³H)-Thymidine incorporation into cellular DNA is measured by liquid scintillation counting.

Accordingly, in one embodiment, antibodies of the invention bind the p40 subunit of IL-12 and/or IL-23 and inhibit phytohemagglutinin blast proliferation in an in vitro phytohemagglutinin blast proliferation assay (PHA assay) with an IC₅₀ of 1×10⁻⁶ M or less. In one embodiment, antibodies of the invention bind the p40 subunit of IL-12 and/or IL-23 and inhibit phytohemagglutinin blast proliferation in an in vitro phytohemagglutinin blast proliferation assay (PHA assay) with an IC₅₀ of 1×10⁻⁷ M or less. In one embodiment, antibodies of the invention, or antigen-binding portions thereof, bind the p40 subunit of IL-12 and/or IL-23 and inhibit phytohemagglutinin blast proliferation in an in vitro PHA assay with an IC₅₀ of 1×10⁻⁸ M or less. In one embodiment, antibodies of the invention, or antigen-binding portions thereof, bind the p40 subunit of IL-12 and/or IL-23 and inhibit phytohemagglutinin blast proliferation in an in vitro PHA assay with an IC₅₀ of 1×10⁻⁹ M or less. In one embodiment, antibodies of the invention, or antigen-binding portions thereof, bind the p40 subunit of IL-12 and/or IL-23 and inhibit phytohemagglutinin blast proliferation in an in vitro PHA assay with an IC₅₀ of 1×10⁻¹⁰ M or less. In one embodiment, antibodies of the invention, or antigen-binding portions thereof, bind the p40 subunit of IL-12 and/or IL-23 and inhibit phytohemagglutinin blast proliferation in an in vitro PHA assay with an IC₅₀ of 1×10⁻¹¹ M or less. In one embodiment, antibodies of the invention, or antigen-binding portions thereof, bind the p40 subunit of IL-12 and/or IL-23 and inhibit phytohemagglutinin blast proliferation in an in vitro PHA assay with an IC₅₀ of 1×10⁻¹² M or less.

The ability of anti-p40 subunit of IL-12/IL-23 antibodies to inhibit the production of IFNγ by PHA blasts (which production is stimulated by IL-12) can be analyzed as follows. Various concentrations of anti-p40 subunit of IL-12/IL-23 antibody are preincubated for 1 hour at 37° C., 5% CO₂ with 200-400 pg/ml hIL-12 in 100 ml RPMI complete medium in a microtiter plate (U-bottom, 96-well, Costar). PHA blast cells are isolated, washed once and resuspended in RPMI complete medium to a cell density of 1×10⁷ cells/ml. PHA blasts (100 μl of 1×10⁶ cells) are added to the antibody/hIL-12 mixture and incubated for 18 hours at 37° C. and 5% CO₂. After incubation, 150 μl of cell free supernatant is withdrawn from each well and the level of human IFNγ produced is measured by ELISA (Endogen Interferon gamma ELISA, Endogen, Cambridge, Mass.).

Accordingly, in other embodiments, antibodies of the invention bind the p40 subunit of IL-12 and/or IL-23 and inhibit IL-12-induced interferon gamma production by human blast cells with an IC₅₀ value of approximately 1.0×10⁻⁸M. In one embodiment, antibodies of the invention bind the p40 subunit of IL-12 and/or IL-23 and inhibit IL-12-induced interferon gamma production by human blast cells with an IC₅₀ value of approximately 1.0×10⁻⁹M. In one embodiment, antibodies of the invention bind the p40 subunit of IL-12 and/or IL-23 and inhibit IL-12-induced interferon gamma production by human blast cells with an IC₅₀ value of approximately 1.0×10⁻¹⁰M. In one embodiment, antibodies of the invention bind the p40 subunit of IL-12 and/or IL-23 and inhibit IL-12-induced interferon gamma production by human blast cells with an IC₅₀ value of approximately 1.0×10⁻¹¹M. In one embodiment, antibodies of the invention bind the p40 subunit of IL-12 and/or IL-23 and inhibit IL-12-induced interferon gamma production by human blast cells with an IC₅₀ value of approximately 1.0×10⁻¹²M.

The ability of anti-p40 subunit of IL-12/IL-23 antibodies to inhibit the activity of IL-23 can be analyzed using known methods and assays, e.g., as known in the art (see, e.g., www.copewithcytokines.de, under IL-23, for description and references to IL-23 proteins, IL-23 assays and IL-12 assays, the contents of which are entirely incorporated herein by reference) and as described herein. For example, human IL-23 has been shown to stimulate the production of IFN-gamma by PHA blast T-cells and memory T-cells, and has also been shown to induce proliferation of both cell types. Accordingly, the ability of anti-p40 subunit of IL-12/IL-23 antibodies to inhibit the production of IFNγ by PHA blasts (which production is stimulated by IL-23) can be analyzed as described above in the context of IL-12. Further, anti-p40 subunit of IL-12/IL-23 antibodies can be evaluated for their ability to inhibit PHA blast proliferation (which proliferation is stimulated by IL-23) as described above in the context of IL-12. Both IL-23 and IL-12 activate the same signaling molecules, including JAK2, TYK2, and STAT1, STAT3, STAT4, and STAT5. STAT4 activation is substantially weaker and different DNA-binding STAT complexes form in response to IL-23 as compared with IL-12. IL-23 binds to the beta-1 subunit, but not to the beta-2 subunit, of the IL-12 receptor, activating one of the STAT proteins, STAT4, in PHA blast T-cells. Accordingly, the ability of anti-p40 subunit of IL-12/IL-23 antibodies to inhibit the activation of STAT4 in PHA blasy T-cells can be analyzed (see, e.g., assays described in Parham et al. Journal of Immunology 168 (11): 5699-5708 2002, the entire contents of which are hereby incorporated by reference herein). Shimozato et al (Immunology 117 (1): 22-28 (2006)) have reported that IL-23 functions and, in particular, IL-23 induced cytokine (e.g., IFN-gamma) production in splenocytes, is inhibited by the p40 subunit of IL-12-p40, which competes for binding to the IL-23 receptors. Accordingly, the ability of anti-p40 subunit of IL-12/IL-23 antibodies to inhibit the activation of cytokines, e.g., IFN-gamma, in splenocytes an be analyzed, e.g., as described in Shimozato et al., the entire contents of which are hereby incorporated herein by reference.

In another embodiment, antibodies of the invention, or antigen-binding portions thereof, have low toxicity. In particular, antibodies, or antigen-binding portions thereof, wherein the individual components, such as the variable region, constant region and framework, individually and/or collectively, possess low immunogenicity, are useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-12 and/or anti-IL-23 antibodies of the invention in patients treated with the same, as occurring in less than 25% of patients treated, preferably, in less than 10% of patients treated with the recommended dose for the recommended course of therapy during the treatment period.

Antibodies of the invention can be tested for binding to the p40 subunit of IL-12 and/or IL-23 (e.g., a portion, domain, site or epitope as described in Section IV(A), IV(C) and/or Table 3 and Table 4 herein) by, for example, standard ELISA. Briefly, microtiter plates are coated with the purified p40 subunit (or a preferred p40 domain) at 0.25 μg/ml in PBS, and then blocked with 5% bovine serum albumin in PBS. Dilutions of antibody (e.g., dilutions of plasma from immunized mice, e.g., mice immunized with the p40 subunit domain) are added to each well and incubated for 1-2 hours at 37° C. The plates are washed with PBS/Tween and then incubated with secondary reagent (e.g., for human antibodies, a goat-anti-human IgG. Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with immunogen. Hybridomas that bind with high avidity to, e.g., the p40 subunit of IL-12 and/or IL-23 (e.g., a portion, domain, site or epitope of the p40 subunit of IL-12 and/or IL-23 as described in Section IV(A), IV(C) and/or Table 3 and Table 4 herein), are subcloned and further characterized. One clone from each hybridoma, which retains the reactivity of the parent cells (by ELISA), can be chosen for making a 5-10 vial cell bank stored at −140° C., and for antibody purification.

To purify anti-p40 subunit of IL-12 and/or IL-23 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using ELISA plates coated with the p40 subunit of IL-12 and/or IL-23 (e.g., a portion, domain, site or epitope of the p40 subunit of IL-12 and/or IL-23 as described in Section IV(A), IV(C) and/or Table 3 and Table 4 herein) as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-p40 subunit of IL-12 and/or IL-23 human IgGs can be further tested for reactivity with the p40 subunit of IL-12 and/or IL-23, or a domain thereof as described herein, by Western blotting. Briefly, the p40 subunit of IL-12 and/or IL-23 (e.g., a portion, domain, site or epitope of the p40 subunit of IL-12 and/or IL-23 as described in Section IV(A), IV(C) and/or Table 3 and Table 4 herein), can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Epitope mapping may be employed to determine the binding site of an antibody or antigen binding fragment thereof of the invention. Several methods are available which further allow the mapping of conformational epitopes. For example, the methods disclosed in Timmerman et al. (Mol Divers. 2004; 8 (2):61-77) may be used. Timmerman et al. were able to successfully map discontinuous/conformational epitopes using two novel techniques, Domain Scan and Matrix Scan. The techniques disclosed in Ansong et al. (J Thromb Haemost. 2006. 4 (4):842-7) may also be used. Ansong et al. used affinity directed mass spectrometry to map the discontinuous epitope recognized by the antibody R8B12. In addition, imaging techniques such as Protein Tomography may be used to visualize antibody or peptide binding to target RTKs. Protein Tomography has been used previously to gain insight into molecular interactions, and was used to show that an inhibitory antibody acted by binding domain III of EGFR thereby locking EGFR into an inflexible and inactive conformation (Lammerts et al. Proc Natl Acad Sci USA. 2008.105 (16):6109-14). More traditional methods such as site-directed mutagenesis may also be applied to map discontinuous epitopes. Amino acid regions thought to participate in a discontinuous epitope may be selectively mutated and assayed for binding to an antibody or antigen binding fragment thereof of the invention. The inability of the antibody to bind when either region is mutated may indicate that binding is dependent upon both amino acid segments. As noted above, some linear epitopes are characterized by particular three-dimensional structures which must be present in order to bind a moiety of the invention. Such epitopes may be discovered by assaying the binding of the antibody when the p40 subunit of IL-12 and/or IL-23 is in its native or folded state and again when the p40 subunit of IL-12 and/or IL-23 is denatured. An observation that binding occurs only in the folded state would indicate that the epitope is either a linear epitope characterized by a particular folded structure or a discontinuous epitope only present in folded protein.

### VI. Pharmaceutical Compositions Comprising Antibodies of the Invention and Pharmaceutical Administration

The antibodies and antibody-portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The antibodies and antibody-portions of the invention can be incorporated into a pharmaceutical composition suitable for parenteral administration. Preferably, the antibody or antibody-portions will be prepared as an injectable solution containing 0.1-250 mg/ml antibody. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 24%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

In a preferred embodiment, the pharmaceutical composition includes the antibody at a dosage of about 0.01 mg/kg-10 mg/kg. More preferred dosages of the antibody include 1 mg/kg administered every other week, or 0.3 mg/kg administered weekly.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies and antibody-portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups; wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention is coformulated with and/or coadministered with one or more additional therapeutic agents that are useful for treating disorders in which IL-12 and/or IL-23 activity is detrimental. For example, an anti-IL-12, anti-IL-23, and/or anti-p40 antibody or antibody portion of the invention may be coformulated and/or coadministered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies. It will be appreciated by the skilled practitioner that when the antibodies of the invention are used as part of a combination therapy, a lower dosage of antibody may be desirable than when the antibody alone is administered to a subject (e.g., a synergistic therapeutic effect may be achieved through the use of combination therapy which, in turn, permits use of a lower dose of the antibody to achieve the desired therapeutic effect).

Interleukins 12 and/or 23 play a critical role in the pathology associated with a variety of diseases involving immune and inflammatory elements. These diseases include, but are not limited to, rheumatoid arthritis, osteoarthritis, juvenile chronic arthritis, Lyme arthritis, psoriatic arthritis, reactive arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, atopic dermatitis, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acquired immunodeficiency syndrome, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjodgren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, lyme disease, discoid lupus erythematosus, male infertility idiopathic or NOS, sperm autoimmunity, multiple sclerosis (all subtypes), insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis and vitiligo. The human antibodies, and antibody portions of the invention can be used to treat autoimmune diseases, in particular those associated with inflammation, including, rheumatoid spondylitis, allergy, autoimmune diabetes, autoimmune uveitis.

Therefore, in certain aspect, the invention provides methods for treating a disease or disorder comprising administering an effective amount of any of the antibodies described herein or a combination thereof, and wherein the antibody or combination of antibodies is effective for ameliorating the disease or disorder. In certain embodiments, the antibody of the invention is administered together with a pharmaceutically acceptable carrier and/or excipients.

Preferably, the antibodies of the invention or antigen-binding portions thereof, are used to treat rheumatoid arthritis, Crohn's disease, multiple sclerosis, insulin dependent diabetes mellitus and psoriasis, as described in more detail below.

A human antibody, or antibody portion, of the invention also can be administered with one or more additional therapeutic agents useful in the treatment of autoimmune and inflammatory diseases. Antibodies of the invention, or antigen binding portions thereof can be used alone or in combination to treat such diseases. It should be understood that the antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent which imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations which are part of this invention can be the antibodies of the present invention and at least one additional agent selected from the lists below. The combination can also include more than one additional agent, e.g., two or three additional agents if the combination is such that the formed composition can perform its intended function.

Thus, in additional embodiments, an antibody of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (G1) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21.sup.st edition, Springhouse Corp., Springhouse, Pa., 2001; Health Professional's Drug Guide 2001, ed., Shannon, Wilson, Stang, Prentice-Hall, Inc, Upper Saddle River, N.J.; Pharmcotherapy Handbook, Wells et al., ed., Appleton & Lange, Stamford, Conn., each entirely incorporated herein by reference).

Preferred combinations are non-steroidal anti-inflammatory drug(s) also referred to as NSAIDS which include drugs like ibuprofen. Other preferred combinations are corticosteroids including prednisolone; the well known side-effects of steroid use can be reduced or even eliminated by tapering the steroid dose required when treating patients in combination with the anti-IL-12 antibodies of this invention. Non-limiting examples of therapeutic agents for rheumatoid arthritis with which an antibody, or antibody portion, of the invention can be combined include the following: cytokine suppressive anti-inflammatory drug(s) (CSAIDs); antibodies to or antagonists of other human cytokines or growth factors, for example, TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, or their ligands including CD 154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7, (U.S. application Ser. No. 08/599,226 filed Feb. 9, 1996), cA2 (Remicade™), CDP 571, anti-TNF antibody fragments (e.g., CDP870), and soluble p55 or p75 TNF receptors, derivatives thereof, (p75TNFRIgG (Enbrel™) or p55TNFR1gG (Lenercept), soluble IL-13 receptor (sIL-13), and also TNF.alpha. converting enzyme (TACE) inhibitors; similarly IL-1 inhibitors (e.g., Interleukin-1-converting enzyme inhibitors, such as Vx740, or IL-1RA etc.) may be effective for the same reason. Other preferred combinations include Interleukin 11, anti-P7s and p-selectin glycoprotein ligand (PSGL). Yet another preferred combination are other key players of the autoimmune response which may act parallel to, dependent on or in concert with IL-12 function; especially preferred are IL-18 antagonists including IL-18 antibodies or soluble IL-18 receptors, or IL-18 binding proteins. It has been shown that. IL-12 and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

The antibodies of the invention, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNF.alpha. or IL-1 (e.g. IRAK, NIK, IKK, p38 or MAP kinase inhibitors), IL-1.beta. converting enzyme inhibitors (e.g., Vx740), anti-P7s, p-selectin glycoprotein ligand (PSGL), TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1 RI, sIL-1RII, sIL-6R, soluble IL-13 receptor (sIL-13)) and antiinflammatory cytokines (e.g. IL-4, IL-10, IL-11, IL-13 and TGF.beta.). Preferred combinations include methotrexate or leflunomide and in moderate or severe rheumatoid arthritis cases, cyclosporine.

Non-limiting examples of therapeutic agents for inflammatory bowel disease with which an antibody, or antibody portion, of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1α monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD90 or their ligands. The antibodies of the invention, or antigen binding portions thereof, may also be combined with agents, such as methotrexate, cyclosporin, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signalling by proinflammatory cytokines such as TNF.alpha. or IL-1 (e.g. IRAK, NIK, IKK, p38 or MAP kinase inhibitors), IL-1.beta. converting enzyme inhibitors (e.g., Vx740), anti-P7s, p-selectin glycoprotein ligand (PSGL), TNF.alpha. converting enzyme inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R, soluble IL-13 receptor (sIL-13)) and antiinflammatory cytokines (e.g. IL-4, IL-10, IL-11, IL-13 and TGFβ).

Preferred examples of therapeutic agents for Crohn's disease in which an antibody or an antigen binding portion can be combined include the following: TNF antagonists, for example, anti-TNF antibodies, D2E7 (U.S. application Ser. No. 08/599,226, filed Feb. 9, 1996), cA2 (Remicade™), CDP 571, anti-TNF antibody fragments (e.g., CDP870), TNFR-Ig constructs (p75TNFRIgG (Enbrel™) and p55TNFRIgG (Lenercept)), anti-P7s, p-selectin glycoprotein ligand (PSGL), soluble IL-13 receptor (sIL-13), and PDE4 inhibitors. Antibodies of the invention or antigen binding portions thereof, can be combined with corticosteroids, for example, budenoside and dexamethasone. Antibodies of the invention or antigen binding portions thereof, may also be combined with agents such as sulfasalazine, 5-aminosalicylic acid and olsalazine, and agents which interfere with synthesis or action of proinflammatory cytokines such as IL-1, for example, IL-1 converting enzyme inhibitors (e.g., Vx740) and IL-Ira. Antibodies of the invention or antigen binding portion thereof may also be used with T cell signaling inhibitors, for example, tyrosine kinase inhibitors 6-mercaptopurines. Antibodies of the invention or antigen binding portions thereof, can be combined with IL-11.

Non-limiting examples of therapeutic agents for multiple sclerosis with which an antibody, or antibody portion, of the invention can be combined include the following: corticosteroids; prednisolone; methylprednisolone; azathioprine; cyclophosphamide; cyclosporine; methotrexate; 4-aminopyridine; tizanidine; interferon-.beta.1a (Avonex; Biogen); interferon-.beta.1 b (Betaseron; Chiron/Berlex); Copolymer 1 (Cop-1; Copaxone; Teva Pharmaceutical Industries, Inc.); hyperbaric oxygen; intravenous immunoglobulin; clabribine; antibodies to or antagonists of other human cytokines or growth factors, for example, TNF, LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80, CD86, CD90 or their ligands. The antibodies of the invention, or antigen binding portions thereof, may also be combined with agents, such as methotrexate, cyclosporine, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNF.alpha. or IL-1 (e.g. IRAK, NIK, IKK, p38 or MAP kinase inhibitors), IL-1.beta. converting enzyme inhibitors (e.g., Vx740), anti-P7s, p-selectin glycoprotein ligand (PSGL), TACE inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1 RI, sIL-1 RII, sIL-6R, soluble IL-13 receptor (sIL-13)) and antiinflammatory cytokines (e.g. IL-4, IL-10, IL-13 and TGFβ).

Preferred examples of therapeutic agents for multiple sclerosis in which the antibody or antigen binding portion thereof can be combined to include interferon-.beta., for example, IFNbeta1a and IFNbeta1b; copaxone, corticosteroids, IL-1 inhibitors, TNF inhibitors, and antibodies to CD40 ligand and CD80.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.01-20 mg/kg, more preferably 1-10 mg/kg, even more preferably 0.3-1 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In one embodiment, the antibodies of the invention are included in the pharmaceutical compositions disclosed in U.S. application Ser. No. 12/625,057 (Patent Publication No. US 2010-0172862A2), the entire contents of which are hereby incorporated by reference herein.

### VII. Uses of the Antibodies of the Invention

Given their ability to bind to IL-12, IL-23, and/or the p40 subunit, antibodies, or portions thereof (e.g., antigen binding portions of fragments thereof), of the invention can be used to detect IL-12, IL-23, and/or the p40 subunit (e.g., in a biological sample, such as serum or plasma), using a conventional immunoassay, such as an enzyme linked immunosorbent assays (ELISA), an radioimmunoassay (RIA) or tissue immunohistochemistry.

Therefore, in another aspect, the invention provides a method for detecting L-12, IL-23, and/or the p40 subunit in a biological sample comprising contacting a biological sample with an antibody, or antibody portion, of the invention and detecting either the antibody (or antibody portion) bound to L-12, IL-23, and/or the p40 subunit or unbound antibody (or antibody portion), to thereby detect L-12, IL-23, and/or the p40 subunit in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵ I, ¹³¹I, ³⁵S or ³H.

Alternative to labeling the antibody, IL-12, IL-23, and/or the p40 subunit can be assayed in biological fluids by a competition immunoassay utilizing, recombinant (“r”) IL-12, and/or rIL-23, and/or the rp40 standards labeled with a detectable substance and an unlabeled anti-IL-12, and/or anti-IL-23, and/or anti-p40 subunit antibody. In this assay, the biological sample, the labeled rIL-12, and/or rIL-23, and/or the rp40 standards and the anti-hIL-12, and/or anti-IL-23, and/or anti-p40 subunit antibody antibody are combined and the amount of labeled rIL-12, and/or rIL-23, and/or the rp40 standard bound to the unlabeled antibody is determined. The amount of IL-12, and/or IL-23, and/or p40 subunit in the biological sample is inversely proportional to the amount of labeled rIL-12, and/or rIL-23, and/or rp40 subunit standard bound to the anti-IL-12, and/or anti-IL-23, and/or anti-p40 antibody, respectively.

The antibodies encompassed by the invention, including Y61 and J695, can also be used to detect IL-12 from species other than humans, in particular IL-12, and/or IL-23, and/or p40 from primates. For example, Y61 can be used to detect IL-12 in the cynomolgus monkey and the rhesus monkey. J695 can be used to detect IL-12 in the cynomolgus monkey, rhesus monkey, and baboon. However, neither antibody cross reacts with mouse or rat IL-12.

The antibodies and antibody portions of the invention are capable of neutralizing the activity of IL-12, IL-23 and/or the p40 subunit of IL-12 and/or IL-23 in vitro, and in vivo. Accordingly, the antibodies and antibody portions of the invention can be used to inhibit IL-12, and/or IL-23, and/or p40 activity, e.g., in a cell culture containing them, in human subjects or in other mammalian subjects having IL-12, and/or IL-23, and/or p40 with which an antibody of the invention cross-reacts (e.g. primates such as baboon, cynomolgus and rhesus). In one embodiment, the invention provides an isolated human antibody, or antigen-binding portion thereof, that neutralizes the activity of human IL-12, IL-23 and/or the p40 subunit of IL-12 and/or IL-23, and at least one additional primate. IL-12, IL-23 and/or p40 subunit of IL-12 and/or IL-23 selected from the group consisting of baboon IL-12, IL-23 and/or p40 subunit of IL-12 and/or IL-23, marmoset IL-12, IL-23 and/or p40 subunit of IL-12 and/or IL-23, chimpanzee IL-12, IL-23 and/or p40 subunit of IL-12 and/or IL-23, cynomolgus IL-12, IL-23 and/or p40 subunit of IL-12 and/or IL-23 and rhesus IL-12, IL-23 and/or p40 subunit of IL-12 and/or IL-23, but which does not neutralize the activity of the mouse IL-12, IL-23 and/or p40 subunit of IL-12 and/or IL-23. Preferably, the IL-12, IL-23 and/or p40 subunit of IL-12 and/or IL-23 is human IL-12, IL-23 and/or p40 subunit of IL-12 and/or IL-23. For example, in a cell culture containing, or suspected of containing human IL-12, IL-23 and/or p40 subunit of human IL-12 and/or IL-23, an antibody or antibody portion of the invention can be added to the culture medium to inhibit human IL-12, IL-23 and/or p40 subunit of human IL-12 and/or IL-23 activity in the culture.

In another embodiment, the invention provides a method for inhibiting the activity of IL-12, IL-23 and/or the p40 subunit of IL-12 and/or IL-23 in a subject suffering from a disorder in which the activity of IL-12, IL-23 and/or the p40 subunit of IL-12 and/or IL-23 is detrimental. IL-12, IL-23 and/or the p40 subunit of IL-12 and/or IL-23 have been implicated in the pathophysiology of a wide variety of disorders (Windhagen et al., (1995) J. Exp. Med. 182: 1985-1996; Morita et al. (1998) Arthritis and Rheumatism. 41: 306-314; Bucht et al., (1996) Clin. Exp. Immunol. 103: 347-367; Fais et al. (1994) J. Interferon Res. 14:235-238; Pyrronchi et al., (1997) Am. J. Path. 150:823-832; Monteleone et al., (1997) Gastroenterology. 112:1169-1178, and Berrebi et al., (1998) Am. J. Path 152:667-672; Pyrronchi et al (1997) Am. J. Path. 150:823-832). The invention provides methods for inhibiting the activity of IL-12, IL-23 and/or the p40 subunit of IL-12 and/or IL-23 in a subject suffering from such a disorder, which method comprises administering to the subject an antibody or antibody portion of the invention such that the activity of IL-12, IL-23 and/or the p40 subunit of IL-12 and/or IL-23 in the subject is inhibited. Preferably, the IL-12, IL-23 and/or p40 subunit of IL-12 and/or IL-23 is human IL-12, IL-23 and/or p40 subunit of IL-12 and/or IL-23 and the subject is a human subject. Alternatively, the subject can be a mammal expressing IL-12, IL-23 and/or p40 subunit of IL-12 and/or IL-23 with which an antibody of the invention cross-reacts. Still further the subject can be a mammal into which has been introduced human IL-12, human IL-23 and/or p40 subunit of human IL-12 and/or IL-23 (e.g., by administration of human IL-12, human IL-23 and/or p40 subunit of human IL-12 and/or IL-23 or by expression of a human IL-12, human IL-23 and/or p40 subunit of human IL-12 and/or IL-23 transgene). An antibody of the invention can be administered to a human subject for therapeutic purposes (discussed further below). Moreover, an antibody of the invention can be administered to a non-human mammal expressing an IL-12, IL-23 and/or p40 subunit of IL-12 and/or IL-23 with which the antibody cross-reacts for veterinary purposes or as an animal model of human disease. Regarding the latter, such animal models may be useful for evaluating the therapeutic efficacy of antibodies of the invention (e.g., testing of dosages and time courses of administration).

As used herein, the phrase “a disorder in which the activity of IL-12, IL-23 and/or the p40 subunit of IL-12 and/or IL-23 is detrimental” is intended to include diseases and other disorders in which the presence of IL-12, IL-23 and/or the p40 subunit of IL-12 and/or IL-23 in a subject suffering from the disorder has been shown to be or is suspected of being either responsible for the pathophysiology of the disorder or a factor that contributes to a worsening of the disorder. Accordingly, a disorder in which the activity of IL-12, IL-23 and/or the p40 subunit of IL-12 and/or IL-23 is detrimental is a disorder in which inhibition of the activity of IL-12, IL-23 and/or the p40 subunit of IL-12 and/or IL-23 is expected to alleviate the symptoms and/or progression of the disorder. Such disorders may be evidenced, for example, by an increase in the concentration of IL-12, IL-23 and/or the p40 subunit of IL-12 and/or IL-23 in a biological fluid of a subject suffering from the disorder (e.g., an increase in the concentration of IL-12, IL-23 and/or the p40 subunit of IL-12 and/or IL-23 in serum, plasma, synovial fluid, etc. of the subject), which can be detected, for example, using an anti-IL-12, anti-IL-23 and/or anti-p40 subunit of IL-12 and/or IL-23 antibody as described above. There are numerous examples of disorders in which the activity of IL-12, IL-23 and/or the p40 subunit of IL-12 and/or IL-23 is detrimental. In one embodiment, the antibodies or antigen binding portions thereof, can be used in therapy to treat the diseases or disorders described herein. In another embodiment, the antibodies or antigen binding portions thereof, can be used for the manufacture of a medicine for treating the diseases or disorders described herein.

In an additional aspect, the invention provides a method for the screening of agents that modulate at least one of the expression, amount, and/or activity of IL-12, IL-23 and/or the p40 subunit of IL-12 and/or IL-23 and/or at least one of the expression, amount, and/or activity of IL-12, IL-23 and/or the p40 subunit of IL-12 and/or IL-23 in a biological sample comprising providing a sample to be tested, e.g., a cell, tissue, organ or individual to be studied; providing an antibody of the invention, wherein the antibody contains a detectable label or is detectable by a second molecule having a detectable label; treating the test sample with a test agent, e.g., a small molecule compound or biopolymer; contacting the test sample with the antibody; and detecting and/or measuring the expression, amount, and/or activity of IL-12, IL-23 and/or the p40 subunit of IL-12 and/or IL-23, and/or the expression, amount, and/or activity of IL-12, IL-23 and/or the p40 subunit of IL-12 and/or IL-23 in the sample, wherein an increase or decrease in at least one of the expression, amount, and/or activity of IL-12, IL-23 and/or the p40 subunit of IL-12 and/or IL-23, and/or increase or decrease in at least one of the expression, amount, and/or activity of IL-12, IL-23 and/or the p40 subunit of IL-12 and/or IL-23 versus that of an untreated sample is indicative of an agent capable of modulating at least one of the expression, amount, and/or activity of the IL-12, IL-23 and/or the p40 subunit of IL-12 and/or IL-23, and/or at least one of the expression, amount, and/or activity of IL-12, IL-23 and/or the p40 subunit of IL-12 and/or IL-23 in the sample.

The use of the antibodies and antibody portions of the invention in the treatment of a few non-limiting specific disorders is discussed further below:

### Rheumatoid Arthritis

Interleukin-12 has been implicated in playing a role in inflammatory diseases such as rheumatoid arthritis. Inducible IL-12p40 message has been detected in synovia from rheumatoid arthritis patients and IL-12 has been shown to be present in the synovial fluids from patients with rheumatoid arthritis (see e.g., Morita et al, (1998) Arthritis and Rheumatism 41: 306-314). IL-12 positive cells have been found to be present in the sublining layer of the rheumatoid arthritis synovium. The human antibodies, and antibody portions of the invention can be used to treat, for example, rheumatoid arthritis, juvenile rheumatoid arthritis, Lyme arthritis, rheumatoid spondylitis, osteoarthritis and gouty arthritis. Typically, the antibody, or antibody portion, is administered systemically, although for certain disorders, local administration of the antibody or antibody portion may be beneficial. An antibody, or antibody portion, of the invention also can be administered with one or more additional therapeutic agents useful in the treatment of autoimmune diseases.

In the collagen induced arthritis (CIA) murine model for rheumatoid arthritis, treatment of mice with an anti-IL-12 mAb (rat anti-mouse IL-12 monoclonal antibody, C17.15) prior to arthritis profoundly suppressed the onset, and reduced the incidence and severity of disease. Treatment with the anti-IL-12 mAb early after onset of arthritis reduced severity, but later treatment of the mice with the anti-IL-12 mAb after the onset of disease had minimal effect on disease severity.

### Crohn's Disease

Interleukin-12 also plays a role in the inflammatory bowel disease, Crohn's disease. Increased expression of FN-.gamma. and IL-12 occurs in the intestinal mucosa of patients with Crohn's disease (see e.g., Fais et al., (1994) J. Interferon Res. 14: 235-238; Pyrronchi et al., (1997) Amer. J. Pathol. 150: 823-832; Monteleone et al., (1997) Gastroenterology 112: 1169-1178; Berrebi et al., (1998) Amer. J. Pathol. 152: 667-672). Anti-IL-12 antibodies have been shown to suppress disease in mouse models of colitis, e.g., TNBS induced colitis IL-2 knockout mice, and recently in IL-10 knock-out mice. Accordingly, the antibodies, and antibody portions, of the invention, can be used in the treatment of inflammatory bowel diseases.

### Multiple Sclerosis

Interleukin-12 has been implicated as a key mediator of multiple sclerosis. Expression of the inducible IL-12 p40 message or IL-12 itself can be demonstrated in lesions of patients with multiple sclerosis (Windhagen et al., (1995) J. Exp. Med. 182: 1985-1996, Drulovic et al., (1997) J. Neurol. Sci. 147:145-150). Chronic progressive patients with multiple sclerosis have elevated circulating levels of IL-12. Investigations with T-cells and antigen presenting cells (APCs) from patients with multiple sclerosis revealed a self-perpetuating series of immune interactions as the basis of progressive multiple sclerosis leading to a Th1-type immune response. Increased secretion of IFN-.gamma. from the T cells led to increased IL-12 production by APCs, which perpetuated the cycle leading to a chronic state of a Th1-type immune activation and disease (Balashov et al., (1997) Proc. Natl. Acad. Sci. 94: 599-603). The role of IL-12 in multiple sclerosis has been investigated using mouse and rat experimental allergic encephalomyelitis (EAE) models of multiple sclerosis. In a relapsing-remitting EAE model of multiple sclerosis in mice, pretreatment with anti-IL-12 mAb delayed paralysis and reduced clinical scores. Treatment with anti-IL-12 mAb at the peak of paralysis or during the subsequent remission period reduced clinical scores. Accordingly, the antibodies or antigen binding portions thereof of the invention may serve to alleviate symptoms associated with multiple sclerosis in humans.

### Insulin-Dependent Diabetes Mellitus

Interleukin-12 has been implicated as an important mediator of insulin-dependent diabetes mellitus (IDDM). IDDM was induced in NOD mice by administration of IL-12, and anti-IL-12 antibodies were protective in an adoptive transfer model of IDDM. Early onset IDDM patients often experience a so-called “honeymoon period” during which some residual islet cell function is maintained. These residual islet cells produce insulin and regulate blood glucose levels better than administered insulin. Treatment of these early onset patients with an anti-IL-12 antibody may prevent further destruction of islet cells, thereby maintaining an endogenous source of insulin.

### Psoriasis

Interleukin-12 has been implicated as a key mediator in psoriasis. Psoriasis involves acute and chronic skin lesions that are associated with a TH 1-type cytokine expression profile. (Hamid et al. (1996) J. Allergy Clin. Immunol. 1:225-231; Turka et al. (1995) Mol. Med. 1:690-699). IL-12 p35 and p40 mRNAs were detected in diseased human skin samples. Accordingly, the antibodies or antigen binding portions thereof of the invention may serve to alleviate chronic skin disorders such psoriasis. The antibodies or antigen binding portions thereof may be used to treat various forms of psoriasis, such as plaque psoriasis and chronic psoriasis. The antibodies or antigen binding portions thereof may also be used to treat psoriasis of varying severity, such as moderate to severe psoriasis.

The present invention is further illustrated by the following examples which should not be construed as limiting in any way. The contents of all cited references, including literature references, issued patents, and published patent applications, as cited throughout this application are hereby expressly incorporated by reference. It should further be understood that the contents of all the tables are incorporated by reference.

### EXAMPLES

The present invention is further illustrated by the following examples, which should not be construed as further limiting. The contents of all figures and all references, patents and published patent applications cited throughout this application, as well as the Figures, are expressly incorporated herein by reference in their entirety.

### Example 1

### Protein Expression and Purification

A. Preparation and Assay of the Human Monoclonal Antibody J695.

J695 was secreted from recombinant Chinese hamster ovary (CHO) cell line ALP905 (see, for example, PCT Publication No. WO0056772 A1) cultured in a 1,000 liter bioreactor. Following removal of CHO cells by filtration, the mAb was purified using cation exchange, anion exchange and hydrophobic interaction chromatography. J695 was concentrated to 71.8 mg/ml in 5 mM L-histidine, 5 mM L-methionine, 0.5% sucrose, 2% D-mannitol, 0.005% polysorbate-80, pH 6.0 and frozen at −80° C. Biacore, PHA blast, and RB assays were performed as described in PCT Publication No. WO0056772 A1, the entire contents of which, are incorporated herein by reference.

B. Preparation of the J695 Fab Fragment.

J695 was diluted to 20 mg/ml with 20 mM phosphate, 2.5 mM cysteine.HCl, 10 mM EDTA, pH 7.0 and then digested in a solution containing 1% immobilized papain (cat. #20341, Pierce Endogen, Rockford, Ill.) and 2.5 mM cysteine.HCl overnight at 37° C. Papain was removed by centrifugation (15 min, 3200 g) and the supernatant, diluted with one part of 20 mM NaH₂PO₄, 150 mM NaCl, pH 7, was passed at 4° C. over a Hi-trap protein A column (cat. #17-0402-03, Amersham Biosciences, Piscataway, N.J.) equilibrated in the same buffer. The Fab was isolated in the flow through, concentrated to 4 mg/ml by centrifugation (cat. # UFV4BGC25, Millipore Corporation, Bedford, Mass.), and dialyzed into 20 mM HEPES, 150 mM NaCl, 0.1 mM EDTA, pH 7.0. The Fab was further concentrated to 55 mg/ml for crystallization. The Fab concentration was determined by UV absorbance at 280 nm in 6 M guanidine.HCl, 20 mM NaH₂PO₄, 150 mM NaCl, pH 7.0 (ε=0.67 M⁻¹cm⁻¹) (Gill, S. C. and P. H. von Hippel (1989). “Calculation of protein extinction coefficients from amino acid sequence data.” Anal. Biochem. 182 (2): 319-326).

C. Preparation of the J695 Fab/IL-12 p70 Complex.

IL-12 p70 was expressed from a stable CHO cell line. Cell supernatants were purified over several columns composed of Q-Sepharose Fast Flow, CM-Sepharose Fast Flow, Phenyl Sepharose High Substitution Fast Flow, Spiral Cartridge Concentrator, and Sephacryl S-200 High Resolution. The final column buffer was PBS pH7.4, which was the final IL-12 p70 storage buffer. The complex with J695 Fab, generated as above, was formed by mixing equal molar amounts of the Fab and IL-12 p70 followed by isolation of the complex by size exclusion chromatography.

### Example 2

### Protein Crystallization

A. Crystallization of J695 Fab in Crystal Form I.

J695 Fab was crystallized using hanging-drop vapor diffusion methods. J695 Fab (1 μl) was mixed with 1 μl of reservoir solution (25% PEG 4000, 0.1 M Na citrate, pH 5.6, 0.2 M (NH₄)₂SO₄) and equilibrated at 18° C. Jewel-like crystals formed in seven days to dimensions of 0.125×0.125×0.05 mm. These crystals are termed herein as “Crystal Form I”.

B. Crystallization of J695 Fab in Crystal Form II.

J695 Fab was crystallized using hanging-drop vapor diffusion methods. J695 Fab (1 μl) was mixed with 1 μl of reservoir solution (12% PEG 4000, 0.1 M Tris, pH 8.5) and equilibrated at 4° C. Tablet-like crystals grew in seven days to dimensions of 0.25×0.05×0.025 mm. These crystals are termed herein as “Crystal Form II”.

C. Crystallization of the J695 Fab/IL-12 p70 Complex.

The J695 Fab/IL-12 p70 complex was crystallized using hanging-drop vapor diffusion methods. Complex (1 μl) was mixed with 1 μl of reservoir solution (16% PEG 4K, 10% 2-propanol, 0.1 M Na HEPES pH 7.5, 0.2 M (NH₄)₂SO₄) and equilibrated at 18° C. Additives in the reservoir (6% dioxane, or 4.3% xylitol) improved diffraction. The crystals were elongated rectangular tablets with etched ends.

### Example 3

### Determination of the Crystal Structure of J695 Fab in Crystal Form I

A. Cryoprotection and Flash Cooling of J695 Fab Form I Crystals.

Form I crystals, grown as described above in the presence of 25% PEG 4000, 0.1 M Na citrate, pH 5.6, 0.2 M (NH₄)₂SO₄, were harvested into mother liquor solutions containing increasing amounts of glycerol (5-15%) and then flash frozen in liquid nitrogen. The crystals were stored in a liquid nitrogen refrigerator until x-ray diffraction data were collected.

B. X-Ray Diffraction Data Collection from an J695 Fab Form I Crystal (Crystal 1).

X-ray diffraction data from an J695 Fab Form I crystal (Crystal 1) were collected by the rotation method to 1.34-Å resolution at beamline X26C (λ=1.1 Å) at the National Synchrotron Light Source (NSLS), Brookhaven National Laboratory, Upton, N.Y., using an ADSC Quantum 210 detector. The Fab crystal was maintained at a temperature of 100 K with an Oxford Cryosystems Cryostream cooler during data collection. For each frame of data (240 total) the crystal was rotated by 0.5°. The data were processed with the HKL2000 suite of programs (Otwinowski, Z. and W. Minor (1997). Processing of X-ray Diffraction Data Collected in Oscillation Mode. New York, Academic Press). After determining the crystal orientation, the data were integrated (in space group P2₁2₁2₁, a=53.92 Å, b=67.36 Å, c=115.79 Å; unit cell information is summarized in Table 5) with DENZO and scaled and merged with SCALEPACK, and placed on an absolute scale and reduced to structure factor amplitudes with TRUNCATE. Further data manipulation was performed with the CCP4 Program Suite (Collaborative Computational Project 4 (1994) “The CCP4 Suite: Programs for Protein Crystallography.” Acta Crystallogr D Biol Crystallogr 50:760-763). Five percent of the unique reflections were assigned, in a random fashion, to the “free” set, for calculation of the free R-factor (R_{free}) (Brunger, A. T. (1992). “The free R value: a novel statistical quantity for assessing the accuracy of crystal structures.” Nature 355: 472-474); the remaining 95% of the reflections constituted the “working” set, for calculation of the R-factor (R). The x-ray diffraction data are summarized in Table 6.

C. Molecular Replacement Solution of the J695 Fab Form I Crystal Structure (Crystal 1).

The structure of J695 Fab in crystal Form I was solved by molecular replacement using CNX (Brunger, A. T., P. D. Adams, et al. (1998). “Crystallography & NMR system (CNS): A new software system for macromolecular structure determination.” Acta Crystallogr. D54: 905-921). Based on the unit cell volumes and the Fab molecular weight (46,608 Da), it was expected that Form I contained 1 Fab per asymmetric unit (45% solvent, Vₘ=2.3 Å³/Da) (Matthews, B. W. (1968). “Solvent content of protein crystals.” J Mol Biol 33: 491-7). Five percent of the randomly selected reflections were used for cross-validation throughout the refinement (Brünger, A. T. (1992). “The free R value: a novel statistical quantity for assessing the accuracy of crystal structures.” Nature 355: 472-474). Out of several homologous Fab search models, only one, with an elbow angle similar to J695 (PDB entry 8fab, (Strong, R. K., R. Campbell, et al. (1991). “Three-dimensional structure of murine anti-p-azophenylarsonate Fab 36-71.1. X-ray crystallography, site-directed mutagenesis, and modeling of the complex with hapten.” Biochemistry 30: 3739-3748), succeeded; rigid body refinement further altered the elbow angle. The translation function indicated that the correct space group was P2₁2₁2₁. Residues not conserved between the search model and J695 were truncated to alanine and the CDRs were removed. Simulated annealing, Powell minimization and group temperature factor refinements were performed using CNX (Brünger, A. T., P. D. Adams, et al. (1998). “Crystallography & NMR system (CNS): A new software system for macromolecular structure determination.” Acta Crystallogr. D54: 905-921). After refinement, the correct side chain atoms and CDR residues were built into regions of positive SigmaA-weighted (Read, R. J. (1986). “Improved Fourier coefficients for maps using phases from partial structures with errors.” Acta Crystallogr. A42: 140-149) Fₒ-F_{c} electron density (2σ) using the visualization program O (Jones, T. A., J. Y. Zou, et al. (1991). “Improved methods for building protein models in electron density maps and the location of errors in these models.” Acta Crystallogr. A47: 110-119). CDR H3 appeared to be disordered and could not be modeled. Alternate side chain conformations were added and the model was refined further in REFMAC (Murshudov, G. N., A. A. Vagin, et al. (1997). “Refinement of macromolecular structures by the maximum-likelihood method.” Acta Crystallogr. D53: 240-255) using anisotropic temperature factors. Water atoms were added and the model was refined to a final R_{cryst}/R_{free} of 16.4/19.7%. The quality of the model was evaluated using Procheck (Laskowski, R. A., M. W. MacArthur, et al. (1993). “PROCHECK: a program to check the stereochemical quality of protein structures.” J. Appl. Crystallogr. 26: 283-291) and Whatcheck (Hooft, R. W. W., G. Vriend, et al. (1996). “Errors in protein structures.” Nature 381: 272). Refinement statistics are reported in Table 7.

### Example 4

### Determination of the Crystal Structure of J695 Fab in Crystal Form II

A. Cryoprotection and Flash Cooling of J695 Fab Form II Crystals.

Form II crystals, grown as described above in the presence of 12% PEG 4000, 0.1 M Tris, pH 8.5, were harvested into mother liquor solutions containing increasing amounts of glycerol (5-15%) and then flash frozen in liquid nitrogen. The crystals were stored in a liquid nitrogen refrigerator until x-ray diffraction data were collected.

B. X-Ray Diffraction Data Collection from an J695 Fab Form II Crystal (Crystal 2)

X-ray diffraction data from an J695 Fab Form II crystal (Crystal 2) were collected by the rotation method to 2.1-Å resolution at beamline X26C (λ=1.1 Å) at the National Synchrotron Light Source (NSLS), Brookhaven National Laboratory, Upton, N.Y., using an ADSC Quantum 210 detector. The Fab crystal was maintained at a temperature of 100 K with an Oxford Cryosystems Cryostream cooler during data collection. For each frame of data (360 total) the crystal was rotated by 0.5°. The data were processed with the HKL2000 suite of programs (Otwinowski, Z. and W. Minor 1997 “Processing of X-ray Diffraction Data Collected in Oscillation Mode” New York, Academic Press). After determining the crystal orientation, the data were integrated (in space group P2₁, a=85.62 Å, b=173.41 Å, c=139.85 Å, β=105.5°; unit cell information is summarized in Table 5) with DENZO and scaled and merged with SCALEPACK, and placed on an absolute scale and reduced to structure factor amplitudes with TRUNCATE. Further data manipulation was performed with the CCP4 Program Suite (Collaborative Computational Project 4 (1994) “The CCP4 Suite: Programs for Protein Crystallography.” Acta Crystallogr D Biol Crystallogr 50:760-763). Five percent of the unique reflections were assigned, in a random fashion, to the “free” set, for calculation of the free R-factor (R_{free}) (Brünger, A. T. 1992 “The free R value: a novel statistical quantity for assessing the accuracy of crystal structures” Nature 355: 472-474); the remaining 95% of the reflections constituted the “working” set, for calculation of the R-factor (R). The x-ray diffraction data are summarized in Table 6.

C. Molecular Replacement Solution of the J695 Fab Form II Crystal Structure (Crystal 2).

The structure of J695 Fab in crystal Form II was solved by molecular replacement. Based on the unit cell volumes and the Fab molecular weight (46,608 Da), it was expected that Form II contained between eight and six Fabs per asymmetric unit (50-63% solvent, Vₘ=2.7-3.6 Å³/Da) (Matthews, B. W. (1968). “Solvent content of protein crystals.” J Mol Biol 33: 491-7). Five percent of the randomly selected reflections were used for cross-validation throughout the refinement (Brünger, A. T. (1992). “The free R value: a novel statistical quantity for assessing the accuracy of crystal structures.” Nature 355: 472-474). Initial attempts to solve the Form II structure, using a largely-refined Form I structure as the search model, were unsuccessful. There appeared to be pseudo-translational symmetry, consistent with off-origin peaks in the native Patterson map, that related pairs of possible solutions, but CNX (Brünger, A. T., P. D. Adams, et al. (1998). “Crystallography & NMR system (CNS): A new software system for macromolecular structure determination.” Acta Crystallogr. D54: 905-921), AMORE (Navaza, J. (1994). “AMoRe: an automated package for molecular replacement.” Acta Crystallog. A50: 157-163) and EPMR (Kissinger, C. R., D. K. Gehlhaar, et al. (2001). EPMR: A program for crystallographic molecular replacement by evolutionary search. La Jolla, Calif., Agouron Pharmaceuticals, Inc) did not provide a definitive solution. MOLREP (Vagin, A. A. and A. Teplyakov (1997). “MOLREP: an automated program for molecular replacement.” J. Appl. Crystallogr. 30: 1022-1025) was able to position eight Fabs, the combination of which resulted in a correlation coefficient of 32.3% and an R-factor of 55.4% at 4 Å in space group P2₁. This solution revealed that two Fabs are aligned in an antiparallel fashion roughly along <011>, related to one another by a pseudo-dyad parallel to [100]. A second Fab pair is arrayed about the same dyad, but displaced by ˜½a. This tetrameric Fab assembly is duplicated by the translational vector [˜½a, ˜½b, ˜½c] to give the other four Fabs in the asymmetric unit.

After rigid body refinement, examination of the SigmaA-weighted maps (Read, R. J. (1986). “Improved Fourier coefficients for maps using phases from partial structures with errors.” Acta Crystallogr. A42: 140-149) revealed disordered constant domains in two Fabs; these domains were removed and the electron density map was subjected to solvent flattening using SOLVE (Terwilliger, T. C. and J. Berenedzen (1999). “Automated MAD and MIR structure solution.” Acta Cryst. D. 55: 849-861). Refinement in REFMAC (Murshudov, G. N., A. A. Vagin, et al. (1997). “Refinement of macromolecular structures by the maximum-likelihood method.” Acta Crystallogr. D53: 240-255) using isotropic B-factors alternated with rebuilding in O (Jones, T. A., J. Y. Zou, et al. (1991). “Improved methods for building protein models in electron density maps and the location of errors in these models.” Acta Crystallogr. A47: 110-119). Constant domains and CDRs were rebuilt into positive electron density (2σ). The two relatively disordered constant domains had average B-factors of ˜75 Å² and ˜85 Å². Water atoms were added and the model was refined to a final R_{cryst}/R_{free} of 19.5/25.9%. The quality of the model was evaluated using Procheck (Laskowski, R. A., M. W. MacArthur, et al. (1993). “PROCHECK: a program to check the stereochemical quality of protein structures.” J. Appl. Crystallogr. 26: 283-291) and Whatcheck (Hooft, R. W. W., G. Vriend, et al. (1996). “Errors in protein structures.” Nature 381: 272). Refinement statistics are reported in Table 7.

D. Analysis of Cis-Trans Peptide Bond Isomers in Antibody Structures in the Protein Data Bank.

It was sought to identify all occurrences of cis-to-trans isomerization of peptide bonds in the Ab structures present in the Protein Data Bank. An extensive search of the Protein Data Bank (as of 28 Mar. 2003), conducted to compile a list of all available Ab structures, yielded 453 entries. The search was aided by the summary list maintained by Dr. Andrew C. R. Martin (http://www.bioinf.org.uk/abs/). Initially, a manual search was performed of this set of 453 Ab structures was performed looking for the CISPEP flag, which is found in the PDB header of structures containing cis-peptide bonds. All Ab structures containing cis-peptide bonds were grouped with related structures. A group consisted of related antibodies (e.g. mutants), an Ab in different ligation states or crystal forms, and multiple copies of an Ab in a single crystal form. The groups were then analyzed manually to determine whether the cis-peptide bond involved a proline residue, and whether the cis-proline found in one group member was conserved or not in the other group members. This analysis was deemed incomplete, however, when it was realized that annotation of the PDB entries by the CISPEP flag was unreliable.

The 453 PDB entries were then re-searched using the following computer algorithm: Measure for all peptide bonds, in all 453 PDB entries, the value of the peptide bond ω torsion angle. A peptide bond was considered cis if ω was 0±20°, otherwise trans. The program MOLEMAN2 was used for this step (Kleywegt, G. J. (1995). MOLEMAN2: manipulation and analysis of PDB files. Uppsala, Sweden, Dept. of Cell and Molecular Biology, Uppsala University, Biomedical Centre, Box 596, SE-751 24).

The amino acid sequence flanking each identified cis peptide bond (in each PDB entry) was extracted (±3 residues for a total of 8, including the 2 residues that define the peptide bond).

This query sequence for each cis peptide bond, in each PDB entry, was compared to all of the 8-residue sequences found in the entire collection of 453 entries. Appropriate corrections handled chain termini and breaks. The search also included the PDB entry from which the query sequence was drawn, to allow for the (common) possibility of multiple copies of an Ig domain in the same crystal structure.

Matches were considered significant if at least 6/8 of the residues were identical, and if the central peptide bond in the matching sequence was trans rather than cis.

Matches determined in this manner represent highly-homologous or identical 8-amino acid sequences that are represented in the set of 453 PDB entries with both a cis and a trans central peptide bond. As expected, several antibodies were found to contain cis-to-trans proline isomerization in the constant domain (J695 contains several cis-prolines in its constant domains that do not exhibit configurational isomerism). The analysis was focused on cis-to-trans proline isomerization within the CDRs.

Visual examination of the cis/trans pairs revealed that only one was unequivocally correct, in addition to J695. This prior example is the single-stranded DNA-binding mAb DNA-1 (PDB entry 1i8m; 2.1-Å resolution), which contains two Fabs in the asymmetric unit (Tanner, J. J., A. A. Komissarov, et al. (2001). “Crystal Structure of an Antigen-binding Fragment Bound to Single-stranded DNA.” J. Mol. Biol. 314: 807-822). The ArgH98^{H3}-ProH99^{H3} peptide bond in Fab1 CDR H3 is trans, while in Fab2 it is cis. In the crystal, a dT₅ oligodeoxynucleotide is bound asymmetrically between the two Fabs, especially by CDRs H3. DNA-1 H3 appears to be more flexible than the other CDRs, as illustrated by the large number of conformations it can adopt within a single crystal form or between multiple crystal forms (Tanner, J. J. (2003). Personal Communication).

The analysis found several antibodies reported to contain cis-to-trans proline isomerization in the CDRs, usually at position 95 of CDR L3. However, a detailed inspection of all the relevant structures invariably revealed structural errors in the region of interest.

### Example 5

### Determination of the Crystal Structure of the J695 Fab/IL-12 p70 Complex

A. Cryoprotection and Flash Cooling of J695 Fab/IL-12 p70 Complex Crystals.

J695 Fab/IL-12 p70 complex crystals, grown as described above in the presence of 16% PEG 4K, 10% 2-propanol, 0.1 M Na HEPES pH 7.5, 0.2 M (NH₄)₂SO₄, were harvested into mother liquor solutions containing increasing amounts of glucose (5-15%) and then flash frozen in liquid nitrogen. The crystals were stored in a liquid nitrogen refrigerator until x-ray diffraction data were collected.

B. X-Ray Diffraction Data Collection from an J695 Fab/IL-12 p70 Complex Crystal (Crystal 3).

X-ray diffraction data from a single J695 Fab/IL-12 p70 complex crystal (Crystal 3) were collected by the rotation method to 3.25-Å resolution at the Industrial Macromolecular Crystallography Association Collaborative Access Team (IMCA-CAT) beamlines 17-BM and 17-ID (λ=1.0 Å), Advanced Photon Source (APS), Argonne National Laboratory, Argonne, Ill., using a MAR CCD detector. The complex crystal was maintained at a temperature of 100 K with an Oxford Cryosystems Cryostream cooler during data collection. For each frame of data (258 total) the crystal was rotated by 0.5°. After determining the crystal orientation, the data were integrated (in space group C222₁, a=136.3151 Å, b=209.5560 Å, c=217.1127 Å; unit cell information is summarized in Table 5) with MOSFLM (Leslie, A. G. W. (1992). “Recent changes to the MOSFLM package for processing film and image plate data.” CCP4 and ESF-EACMB Newsletter on Protein Crystallography 26) and scaled and merged with SCALA (Evans, P. R. (1997). “SCALA.” Joint CCP4 and ESF-EACBM Newsletter 33: 22-24), and placed on an absolute scale and reduced to structure factor amplitudes with TRUNCATE. Further data manipulation was performed with the CCP4 Program Suite (Collaborative Computational Project 4 (1994) “The CCP4 Suite: Programs for Protein Crystallography.” Acta Crystallogr D Biol Crystallogr 50:760-763). Five percent of the unique reflections were assigned, in a random fashion, to the “free” set, for calculation of the free R-factor (R_{free}) (Brünger, A. T. (1992). “The free R value: a novel statistical quantity for assessing the accuracy of crystal structures.” Nature 355: 472-474); the remaining 95% of the reflections constituted the “working” set, for calculation of the R-factor (R). The x-ray diffraction data are summarized in Table 6.

C. Molecular Replacement Solution of the J695 Fab/IL-12 p70 Complex Crystal Structure (Crystal 3).

The structure of the J695 Fab/IL-12 p70 complex was solved by molecular replacement. Based on the unit cell volumes and the Fab and IL-12 p70 molecular weights (46,608 and ˜70,000 Da), it was expected that the crystal contained two Fab/p70 complexes per asymmetric unit (˜61% solvent, Vₘ˜3.3 Å³/Da) (Matthews, B. W. (1968). “Solvent content of protein crystals.” J Mol Biol 33:491-7). The self-rotation function showed two non-crystallographic two-fold rotation axes, with polar rotation angles [θ,φ,χ] equal to [9.77, 90.00, 180.00] and [80.23, 90.00, 180.00], each approximately one-third as strong as the crystallographic two-fold axes, consistent with a non-crystallographic dimer oriented with the two-fold axis ˜10° offset from the crystallographic c axis toward the b axis. There appeared to be no pseudo-translational symmetry, consistent with the lack of off-origin peaks in the native Patterson map. Initial attempts to solve the structure using CNX (Brünger, A. T., P. D. Adams, et al. (1998). “Crystallography & NMR system (CNS): A new software system for macromolecular structure determination.” Acta Crystallogr. D54:905-921), AMORE (Navaza, J. (1994). “AMoRe: an automated package for molecular replacement.” Acta Crystallog. A50:157-163), EPMR (Kissinger, C. R., D. K. Gehlhaar, et al. (2001). EPMR: A program for crystallographic molecular replacement by evolutionary search. La Jolla, Calif., Agouron Pharmaceuticals, Inc), and MOLREP (Vagin, A. A. and A. Teplyakov (1997). “MOLREP: an automated program for molecular replacement.” J. Appl. Crystallogr. 30:1022-1025) were unsuccessful. The structure of the J695 Fab/IL-12 p70 complex was ultimately solved with PHASER (Storoni, L. C., A. J. McCoy, et al. (2004). “Likelihood-enhanced fast rotation functions.” Acta Crystallogr D Biol Crystallogr 60 (Pt 3):432-8) in space group C222₁, using the (refined) J695 Fab Form I and the IL-12 p70 (PDB entry 1f45; (Yoon, C., S. C. Johnston, et al. (2000). “Charged residues dominate a unique interlocking topography in the heterodimeric cytokine interleukin-12.” The EMBO Journal 19 (14):3530-3521) coordinates as the search models. First, two copies of the Fab were placed, providing a clearly-correct log-likelihood gain (LLG) of 1250. Placement of the IL-12 p70 molecules alone was more problematic, producing equivocal results (LLG 130, just one molecule; a second p70 molecule could not be located). With the two Fabs placed as determined above, searching for p70 in addition provided a much improved LLG (2150), consistent with a correct solution. This unequivocal placement of p70 was also consistent with the equivocal placement determined above when p70 was used alone.

Rigid body refinement was carried out using REFMAC (Murshudov, G. N., A. A. Vagin, et al. (1997). “Refinement of macromolecular structures by the maximum-likelihood method.” Acta Crystallogr. D53: 240-255). Five percent of the randomly selected reflections were used for cross-validation throughout the refinement (Brünger, A. T. (1992). “The free R value: a novel statistical quantity for assessing the accuracy of crystal structures.” Nature 355: 472-474). Using data from 20-4.0 Å resolution, ten domains (each Fab immunoglobulin [Ig] domain, and IL-12 p40 and p35) were refined to R_{free}/R=0.401/0.413. Examination of the SigmaA-weighted maps (Read, R. J. (1986). “Improved Fourier coefficients for maps using phases from partial structures with errors.” Acta Crystallogr. A42: 140-149) revealed two Fab molecules placed back-to-back, with one Fab combining site bound predominantly to IL-12 p40 domain 1 (the N-terminal domain). The maps also showed density for the second IL-12 molecule.

PHASER was re-run, with the rigid body-refined model held fixed, searching for the second IL-12 p70. This process was successful, providing an improved LLG of 2926. Refinement within PHASER gave a final LLG of 3562. Repeating the rigid body refinement with REFMAC, now with 16 domains (8 Fab Ig domains, six p40 Ig-like domains, and two p35 domains), provided R_{free}/R=0.400/0.409 (20-3.5 Å). Continued positional refinement (REFMAC) using isotropic B-factors alternated with rebuilding in O (Jones, T. A., J. Y. Zou, et al. (1991). “Improved methods for building protein models in electron density maps and the location of errors in these models.” Acta Crystallogr. A47: 110-119) provided a final R_{free}/R=0.287/0.216. The quality of the model was evaluated using Procheck (Laskowski, R. A., M. W. MacArthur, et al. (1993). “PROCHECK: a program to check the stereochemical quality of protein structures.” J. Appl. Crystallogr. 26: 283-291) and Whatcheck (Hooft, R. W. W., G. Vriend, et al. (1996). “Errors in protein structures.” Nature 381: 272). Refinement statistics are reported in Table 7.

TABLE 5
Summary of Crystallographic Unit Cell Information for
J695 Fab and J695 Fab/IL-12 p70 Complex Crystals.

| | Space | a | b | c | β |
|---|---|---|---|---|---|
| Crystal | Group | (Å) | (Å) | (Å) | (°) |
| 1 | P2₁2₁2₁ | 53.92 | 67.36 | 115.79 | 90 |
| 2 | P2₁ | 85.62 | 173.41 | 139.85 | 105.5 |
| 3 | C222₁ | 136.32 | 209.56 | 217.11 | 90 |

TABLE 6
Summary of X-ray Diffraction Data Statistics for
J695 Fab and J695 Fab/IL-12 p70 Complex Crystals.

| | Space | Resolution | Unique | R_{sym} | | Coverage | |
|---|---|---|---|---|---|---|---|
| Crystal | Group | (Å) * | Reflections | (%) * | <I/σ_{I}> * | (%) * | Multiplicity * |
| 1 | P2₁2₁2₁ | 20-1.34 | 93,561 | 4.4 | 27.9 | 98.0 | ~4.5 |
| | | (1.39-1.34) | | (60.9) | (2.1) | (87.2) | (~2.0) |
| 2 | P2₁ | 20-2.1 | 228,888 | 11.6 | 11.6 | 100 | 3.8 |
| | | (2.15-2.095) | | (73.8) | (1.8) | (100) | (3.8) |
| 3 | C222₁ | 30-3.25 | 43,561 | 13.8 | 10.2 | 88.5 | 7.5 |
| | | (3.33-3.25) | | (49.2) | (2.0) | (53.8) | 3.4 |

* Highest resolution shell in parentheses.

TABLE 7
Summary of Crystallographic Refinement Statistics for
J695 Fab and J695 Fab/IL-12 p70 Complex Crystals.

| | Space | Resolution | R_{free} | R |
|---|---|---|---|---|
| Crystal | Group | (Å) | (%) | (%) |
| 1 | P2₁2₁2₁ | 20-1.34 | 19.7 | 16.4 |
| 2 | P2₁ | 20-2.1 | 25.9 | 19.5 |
| 3 | C222₁ | 20-3.25 | 28.7 | 21.6 |

### INCORPORATION BY REFERENCE

The contents of all cited references (including literature references, patents, patent applications, and websites) that are cited throughout this application, as well as the Figures, are hereby expressly incorporated by reference in their entirety. The practice of the present invention will employ, unless otherwise indicated, conventional techniques of antibody production, which are well known in the art.

### EQUIVALENTS

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. An isolated antibody that binds to the p40 subunit of IL-12 and/or IL-23, or an antigen binding portion thereof, wherein said antibody binds to a portion of the p40 subunit comprising at least one amino acid residue selected from residues 1-197 of the amino acid sequence of SEQ ID NO: 3, or within 1-10 Å of said amino acid residue.

2. The isolated antibody of claim 1, or antigen binding portion thereof, wherein said antibody binds to a portion of the p40 subunit comprising at least one amino acid residue selected from residues 1-107 of the amino acid sequence of SEQ ID NO: 3, or within 1-10 Å of said amino acid residue.

3. The isolated antibody of claim 1, or antigen binding portion thereof, wherein said antibody binds to a portion of the p40 subunit comprising at least one amino acid residue of loops 1-7 of the p40 subunit, wherein the at least one amino acid residue is selected from the group consisting of residues 14-23, 58-60, 84-107, 124-129, 157-164 and 194-197 of the amino acid sequence of SEQ ID NO: 3, or within 1-10 Å of said amino acid residue.

4. The isolated antibody of claim 3, or antigen binding portion thereof, wherein said antibody binds to a portion of the p40 subunit comprising at least one amino acid residue of loop 1 selected from the group consisting of residues 14-23, or within 1-10 Å of said amino acid residue.

5. The isolated antibody of claim 4, or antigen binding portion thereof,
wherein said antibody binds to a portion of the p40 subunit comprising at least one amino acid residue of loop 1 selected from the group consisting of residues 14-18, or within 1-10 Å of said amino acid residue;
wherein said antibody binds to a portion of the p40 subunit comprising at least one amino acid residue of loop 1 selected from the group consisting of residues 14-17, or within 1-10 Å of said amino acid residue; or
wherein said antibody binds to a portion of the p40 subunit comprising at least one amino acid residue of loop 1 selected from the group consisting of residues 15-17, or within 1-10 Å of said amino acid residue.

6. The isolated antibody of claim 3, or antigen binding portion thereof, wherein said antibody binds to a portion of the p40 subunit comprising at least one amino acid residue of loop 2 selected from the group consisting of residues 58-60, or within 1-10 Å of said amino acid residue.

7. The isolated antibody of claim 3, or antigen binding portion thereof, wherein said antibody binds to a portion of the p40 subunit comprising at least one amino acid residue of loop 3 selected from the group consisting of residues 84-94, or within 1-10 Å of said amino acid residue.

8. The isolated antibody of claim 7, or antigen binding portion thereof,
wherein said antibody binds to a portion of the p40 subunit comprising at least one amino acid residue of loop 3 selected from the group consisting of residues 85-93, or within 1-10 Å of said amino acid residue;
wherein said antibody binds to a portion of the p40 subunit comprising at least one amino acid residue of loop 3 selected from the group consisting of residues 86-89 and 93, or within 1-10 Å of said amino acid residue;
wherein said antibody binds to a portion of the p40 subunit comprising at least one amino acid residue of loop 3 selected from the group consisting of residues 86, 87, 89 and 93, or within 1-10 Å of said amino acid residue; or
wherein said antibody binds to a portion of the p40 subunit comprising amino acid residue 87 of loop 3, or within 1-10 Å of said amino acid residue.

9. The isolated antibody of claim 3, or antigen binding portion thereof,
wherein said antibody binds to a portion of the p40 subunit comprising at least one amino acid residue of loop 4 selected from the group consisting of residues 95-107, or within 1-10 Å of said amino acid residue; or
wherein said antibody binds to a portion of the p40 subunit comprising at least one amino acid residue of loop 4 selected from the group consisting of residues 102-104, or within 1-10 Å of said amino acid residue.

10. The isolated antibody of claim 3, or antigen binding portion thereof,
wherein said antibody binds to a portion of the p40 subunit comprising at least one amino acid residue of loop 5 selected from the group consisting of residues 124-129, or within 1-10 Å of said amino acid residue;
wherein said antibody binds to a portion of the p40 subunit comprising at least one amino acid residue of loop 6 selected from the group consisting of residues 157-164, or within 1-10 Å of said amino acid residue; or
wherein said antibody binds to a portion of the p40 subunit comprising at least one amino acid residue of loop 7 selected from the group consisting of residues 194-197, or within 1-10 Å of said amino acid residue.

11. The isolated antibody of claim 3, or antigen binding portion thereof, wherein said antibody binds to a portion of the p40 subunit comprising at least one amino acid residue of loops 1-4 selected from the group consisting of residues 14-23, 58-60, 84-94 and 95-107, or within 1-10 Å of said amino acid residue.

12. The isolated antibody of claim 11, or antigen binding portion thereof,
wherein said antibody binds to a portion of the p40 subunit comprising at least one amino acid residue of loops 1-4 selected from the group consisting of residues 14-18, 85-93 and 102-104, or within 1-10 Å of said amino acid residue;
wherein said antibody binds to a portion of the p40 subunit comprising at least one amino acid residue of loops 1-4 selected from the group consisting of residues 14-17, 86-89, 93 and 103-104, or within 1-10 Å of said amino acid residue; or
wherein said antibody binds to a portion of the p40 subunit comprising at least one amino acid residue of loops 1-4 selected from the group consisting of residues 15-17, 86-87, 89, 93 and 104, or within 1-10 Å of said amino acid residue.

13. The isolated antibody of claim 3, or antigen binding portion thereof, wherein said antibody binds to a portion of the p40 subunit comprising at least one amino acid residue of loops 1-2 selected from the group consisting of residues 14-23 and 58-60, or within 1-10 Å of said amino acid residue.

14. The isolated antibody of claim 13, or antigen binding portion thereof,
wherein said antibody binds to a portion of the p40 subunit comprising at least one amino acid residue of loops 1-2 selected from the group consisting of residues 15, 17-21, 23 and 58-60, or within 1-10 Å of said amino acid residue; or
wherein said antibody binds to a portion of the p40 subunit comprising at least one amino acid residue of loop 1 selected from the group consisting of residues 14-23 and at least one amino acid residue of loop 2 selected from the group consisting of residues 58-60, or within 1-10 Å of said amino acid residue.

15. The isolated antibody of claim 3, or antigen binding portion thereof,
wherein said antibody binds to a portion of the p40 subunit comprising at least one amino acid residue of loops 1 and 3 selected from the group consisting of residues 14-23 and 84-94, or within 1-10 Å of said amino acid residue; or
wherein said antibody binds to a portion of the p40 subunit comprising at least one amino acid residue of loop 1 selected from the group consisting of residues 14-23 and at least one amino acid residue of loop 3 selected from the group consisting of residues 84-94, or within 1-10 Å of said amino acid residue.

16. The isolated antibody of claim 3, or antigen binding portion thereof,
wherein said antibody binds to a portion of the p40 subunit comprising at least one amino acid residue of loops 1 and 4 selected from the group consisting of residues 14-23 and 95-107, or within 1-10 Å of said amino acid residue; or
wherein said antibody binds to a portion of the p40 subunit comprising at least one amino acid residue of loop 1 selected from the group consisting of residues 14-23 and at least one amino acid residue of loop 4 selected from the group consisting of residues 95-107, or within 1-10 Å of said amino acid residue.

17. The isolated antibody of claim 3, or antigen binding portion thereof,
wherein said antibody binds to a portion of the p40 subunit comprising at least one amino acid residue of loops 3 and 4 selected from the group consisting of residues 84-94 and 95-107, or within 1-10 Å of said amino acid residue; or
wherein said antibody binds to a portion of the p40 subunit comprising at least one amino acid residue of loop 3 selected from the group consisting of residues 84-94 and at least one amino acid residue of loop 4 selected from the group consisting of residues 95-107, or within 1-10 Å of said amino acid residue.

18. An isolated antibody, or antigen-binding portion thereof, that competes for binding with the antibody, or antigen binding portion thereof, of claim 1.

19. The isolated antibody, or antigen binding portion thereof, of claim 1, which is not the antibody Y61 or J695.

20. An isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein any one of the variable region residues other than amino acid residues 27, 32, 52, 53, 97, 101 and 102 of SEQ ID NO: 1 and amino acid residues 35, 51 and 90-101 of SEQ ID NO: 2 are independently substituted with a different amino acid.

21. An isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein one or more of the variable region amino acid residues 27, 32, 52, 53, 97, 101 and 102 of SEQ ID NO: 1 and 35, 51 and 90-101 of SEQ ID NO: 2 are independently substituted with a different amino acid residue.

22. The isolated antibody of claim 21, or antigen binding portion thereof,
wherein one or more of the variable region amino acid residues 27, 32 and 102 of SEQ ID NO: 1 are independently substituted with an aromatic residue;
wherein one or more of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln;
wherein one or more of the variable region amino acid residues 92 and 97 of SEQ ID NO: 2 are independently substituted with an aromatic amino acid residue;
wherein one or more of the variable region amino acid residues 101 of SEQ ID NO: 1 and 51 of SEQ ID NO: 2 are independently substituted with an amino acid residue selected from the group consisting of Tyr, Ser, Thr, Asn and Gln;
wherein the variable region amino acid residue 91 of SEQ ID NO: 2 is substituted with any amino acid residue except Gln;
wherein the variable region amino acid residue 95 of SEQ ID NO: 2 is substituted with a different aromatic amino acid residue;
wherein the variable region amino acid residue 97 of SEQ ID NO: 2 is substituted with an amino acid residue selected from the group consisting of Phe, Tyr, Trp, His, Asp, Glu, Asn and Gln;
wherein one or more of the variable region amino acid residues 90-101 of SEQ ID NO: 2 is independently substituted with at least one or more different amino acids, and wherein the length of CDRL3 of the antibody is greater than or equal to 12 amino acid residues;
wherein the antibody has one or more of the following substitutions:
(a) one or more of the variable region amino acid residues 90-101 of SEQ ID NO: 2 is independently substituted with at least one or more different amino acids, and wherein the length of CDRL3 of the antibody is greater than or equal to 12 amino acid residues;
(b) variable region amino acid residue 91 of SEQ ID NO: 2 is substituted with any amino acid residue except Gln;
(c) variable region amino acid residue 95 of SEQ ID NO: 2 is substituted with a different aromatic amino acid residue; or
(d) variable region amino acid residue 97 of SEQ ID NO: 2 is substituted with an amino acid residue selected from the group consisting of Phe, Tyr, Trp, His, Asp, Glu, Asn and Gln; or
wherein one or more of the variable region amino acid residues 52 and 53 of SEQ ID NO: 1 is independently substituted with an amino acid residue selected from the group consisting of Tyr, Ser, Thr, Asn, Gln, Lys and Arg.

23. An isolated antibody that binds to the p40 subunit of IL-12 and/or IL-23, or antigen binding portion thereof, wherein said antibody comprises the heavy chain variable region amino acid sequence of SEQ ID NO: 1 and the light chain variable region amino acid sequence of SEQ ID NO: 2, wherein one or more of the variable region amino acid residues 33, 50, 57 and 99 of SEQ ID NO: 1 and 33 of SEQ ID NO: 2 are independently substituted with a different amino acid residue.

24. The isolated antibody of claim 23, or antigen binding portion thereof,
wherein variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys;
wherein variable region amino acid residue 50 of SEQ ID NO: 1 is substituted with an amino acid residue selected from the group consisting of Phe, Tyr, Trp, His, Met, Gln, Arg and Lys;
wherein variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln;
wherein variable region amino acid residue 99 of SEQ ID NO: 1 is substituted with an amino acid residue selected from the group consisting of Phe, Tyr, Trp, His, Met, Arg and Lys; or
wherein variable region amino acid residue 33 of SEQ ID NO: 2 is substituted with an amino acid residue selected from the group consisting of Phe, Tyr, Trp, His, Gln and Lys.

25. The isolated antibody of claim 24, or antigen binding portion thereof,
wherein variable region amino acid residue 33 of SEQ ID NO: 1 is substituted with Lys;
wherein variable region amino acid residue 50 of SEQ ID NO: 1 is substituted with Tyr or Trp;
wherein variable region amino acid residue 57 of SEQ ID NO: 1 is substituted with Ile or Trp;
wherein variable region amino acid residue 57 of SEQ ID NO: 1 is substituted with Ser or Thr;
wherein variable region amino acid residue 99 of SEQ ID NO: 1 is substituted with Tyr or Trp; or
wherein variable region amino acid residue 33 of SEQ ID NO: 2 is substituted with Tyr or Trp.

26. The antibody, or antigen binding portion thereof, of any one of claims 20, 21 and 23, which is not the antibody J695 or Y61.

27. An isolated antibody, or antigen-binding portion thereof, that competes for binding with the antibody, or antigen binding portion thereof, of any one of claims 20, 21 and 23.

28. A method for altering the activity of an isolated antibody that binds to the p40 subunit of IL-12 and/or IL-23, or antigen binding portion thereof, wherein said antibody or antigen binding portion thereof comprises the heavy chain variable region amino acid sequence of SEQ ID NO: 1 and the light chain variable region amino acid sequence of SEQ ID NO: 2, comprising independently substituting one or more of the variable region amino acid residues 27, 32, 52, 53, 97, 101 and 102 of SEQ ID NO: 1 and amino acid residues 35, 51 and 90-101 of SEQ ID NO 2 with a different amino acid residue, thereby altering the activity of an antibody that binds to the p40 subunit of IL-12 and/or IL-23, or antigen binding portion thereof.

29. The method of claim 28,
wherein one or more of the variable region amino acid residues 27, 32 and 102 of SEQ ID NO: 1 are independently substituted with an aromatic residue;
wherein one or more of the variable region amino acid residues 97 of SEQ ID NO: 1 and 35 and 92 of SEQ ID NO: 2 are independently substituted with an amino acid residue selected from the group consisting of Lys, Arg, Tyr, Asn and Gln;
wherein one or more of the variable region amino acid residues 92 and 97 of SEQ ID NO: 2 are independently substituted with an aromatic amino acid residue;
wherein one or more of the variable region amino acid residues 101 of SEQ ID NO: 1 and 51 of SEQ ID NO: 2 are independently substituted with an amino acid residue selected from the group consisting of Tyr, Ser, Thr, Asn and Gln;
wherein the variable region amino acid residue 91 of SEQ ID NO: 2 is substituted with any amino acid residue except Gln;
wherein the variable region amino acid residue 95 of SEQ ID NO: 2 is substituted with a different aromatic amino acid residue;
wherein the variable region amino acid residue 97 of SEQ ID NO: 2 is substituted with an amino acid residue selected from the group consisting of Phe, Tyr, Trp, His, Asp, Glu, Asn and Gln;
wherein one or more of the variable region amino acid residues 90-101 of SEQ ID NO: 2 are independently substituted with at least one or more different amino acids, and wherein the length of CDRL3 of the antibody is greater than or equal to 12 amino acid residues;
wherein the antibody, or antigen binding portion thereof, has one or more of the following substitutions:
(a) one or more of the variable region amino acid residues 90-101 of SEQ ID NO: 2 are independently substituted with at least one or more different amino acids, and wherein the length of CDRL3 of the antibody is greater than or equal to 12 amino acid residues;
(b) variable region amino acid residue 91 of SEQ ID NO: 2 is substituted with any amino acid residue except Gln;
(c) variable region amino acid residue 95 of SEQ ID NO: 2 is substituted with a different aromatic amino acid residue; or
(d) variable region amino acid residue 97 of SEQ ID NO: 2 is substituted with an amino acid residue selected from the group consisting of Phe, Tyr, Trp, His, Asp, Glu, Asn and Gln; or
wherein one or more of the variable region amino acid residues 52 and 53 of SEQ ID NO: 1 are independently substituted with an amino acid residue selected from the group consisting of Tyr, Ser, Thr, Asn, Gln, Lys and Arg.

30. A method for altering the activity of an isolated antibody that binds to the p40 subunit of IL-12 and/or IL-23, or antigen binding portion thereof, wherein said antibody or antigen binding portion thereof comprises the heavy chain variable region amino acid sequence of SEQ ID NO: 1 and the light chain variable region amino acid sequence of SEQ ID NO: 2, comprising independently substituting one or more of the variable region amino acid residues 33, 50, 57 and 99 of SEQ ID NO: 1 and 33 of SEQ ID NO: 2 with a different amino acid residue, thereby altering the activity of an antibody that binds to the p40 subunit of IL-12 and/or IL-23, or antigen binding portion thereof.

31. The method of claim 30,
wherein variable region amino acid residue 33 of SEQ ID NO: 1 is substituted with an amino acid residue selected from the group consisting of Phe, Tyr, Trp, His, Met, Val, Leu, Ile, Pro, Ala, Ser, Thr, Asn, Gln, Arg and Lys;
wherein variable region amino acid residue 50 of SEQ ID NO: 1 is substituted with an amino acid residue selected from the group consisting of Phe, Tyr, Trp, His, Met, Gln, Arg and Lys;
wherein variable region amino acid residue 57 of SEQ ID NO: 1 is substituted with an amino acid residue selected from the group consisting of Phe, Tyr, Trp, His, Met, Val, Leu, Ile, Pro, Ala, Ser, Thr, Asp, Glu, Asn and Gln;
wherein variable region amino acid residue 99 of SEQ ID NO: 1 is substituted with an amino acid residue selected from the group consisting of Phe, Tyr, Trp, His, Met, Arg and Lys; or
wherein variable region amino acid residue 33 of SEQ ID NO: 2 is substituted with an amino acid residue selected from the group consisting of Phe, Tyr, Trp, His, Gln and Lys.

32. The method of claim 31,
wherein variable region amino acid residue 33 of SEQ ID NO: 1 is substituted with Lys;
wherein variable region amino acid residue 50 of SEQ ID NO: 1 is substituted with Tyr or Trp;
wherein variable region amino acid residue 57 of SEQ ID NO: 1 is substituted with Ile or Trp;
wherein variable region amino acid residue 57 of SEQ ID NO: 1 is substituted with Ser or Thr;
wherein variable region amino acid residue 99 of SEQ ID NO: 1 is substituted with Tyr or Trp; or
wherein variable region amino acid residue 33 of SEQ ID NO: 2 is substituted with Tyr or Trp.

33. An isolated antibody, or antigen binding portion thereof, produced according to the method of claim 28 or 30.

34. An isolated antibody that binds to the p40 subunit of IL-12 and/or IL-23, or antigen binding portion thereof, wherein said antibody binds to a conformational epitope comprising at least one amino acid residue selected from the group consisting of amino acid residues 16, 87 and 93 of the amino acid sequence of SEQ ID NO:3, or within 10 Å of said amino acid residue.

35. The isolated antibody of claim 34, or antigen binding portion thereof, wherein said antibody binds to amino acid residue 16.

36. The isolated antibody, or antigen binding portion thereof, of any one of claims 1, 20, 21, 23 and 34, wherein said antibody binds to the p40 subunit of IL-12 and/or IL-23 with a K_{off} of 1×10⁻³ M⁻¹ or less or a K_{d} of 1×10⁻¹⁰ M or less.

37. The isolated antibody, or antigen binding portion thereof, of any one of claims 1, 20, 21, 23 and 34, wherein said antibody neutralizes the biological activity of the p40 subunit of Il-12 and/or IL-23.

38. A pharmaceutical composition comprising the antibody of claim 37, or antigen binding portion thereof, and a pharmaceutical acceptable carrier or excipients.

39. The pharmaceutical composition of claim 38, further including at least one additional biologically active agent.

40. An isolated nucleic acid that encodes an antibody, or antigen binding portion thereof, of any one of claims 1, 20, 21, 23 and 34.

41. An isolated nucleic acid vector comprising the nucleic acid of claim 40 operably linked with at least one transcription regulatory nucleic acid sequence.

42. A host cell comprising the nucleic acid vector of claim 41.

43. The host cell of claim 42, wherein the host cell is a eukaryotic host cell or prokaryotic host cell.

44. A method for diagnosing at least one IL-12 and/or IL-23 related condition in a subject, comprising contacting a biological sample from said subject with an antibody of any one of claims 1, 20, 21, 23 and 34 or antigen binding portion thereof, and measuring the amount of p40 subunit of IL-12 and/or IL-23 that is present in the sample, wherein the detection of elevated or reduced levels of the p40 subunit of IL-12 and/or IL-23 in the sample, as compared to a normal or control, is indicative of the presence or absence of an IL-12 and/or IL-23 related condition, thereby diagnosing at least one IL-12 and/or IL-23 related condition in the subject.

45. The method of claim 44, wherein the antibody or antigen binding portion thereof contains a detectable label or is detected by a second molecule having a detectable label.

46. A method for identifying an agent that modulates at least one of the expression, level, and/or activity of IL-12 and/or IL-23 in a biological sample, comprising contacting said sample with an antibody of any one of claims 1, 20, 21, 23 and 34, or antigen binding portion thereof, and detecting the expression, level, and/or activity of IL-12 and/or IL-23 in the sample, wherein an increase or decrease in at least one of the expression, level, and/or activity of IL-12 and/or IL-23 compared to an untreated sample is indicative of an agent capable of modulating at least one of the expression, level, and/or activity of IL-12 and/or IL-23, thereby identifying an agent that modulates at least one of the expression, level and/or activity of IL-12 and/or IL-23 in the sample.

47. The method of claim 46, wherein the antibody or antigen binding portion thereof contains a detectable label or is detectable by a second molecule having a detectable label.

48. A method for inhibiting the activity of IL-12 and/or IL-23 in a subject suffering from a disorder in which the activity of IL-12 and/or IL-23 is detrimental, comprising administering to the subject an antibody of any one of claims 1, 20, 21, 23 and 34, or antigen binding portion thereof, such that the activity of IL-12 and/or IL-23 in the subject is inhibited.

49. A method for treating a subject suffering from a disorder in which the activity of IL-12 and/or IL-23 is detrimental, comprising administering to the subject an antibody of any one of claims 1, 20, 21, 23 and 34, or antigen binding portion thereof, thereby treating the subject.
